SEQUENCE LISTING (196 sequences)

SEQ ID NO: 1 - protein, 19 aa, Artificial Sequence
  Description of Artificial Sequence Synthetic peptide
  MOD_RES (14)..(14): Citrullinated Arginine
YQVKPYHGGGAPLRVELPT

SEQ ID NO: 2 - protein, 15 aa, Artificial Sequence
  Description of Artificial Sequence Synthetic peptide
  MOD_RES (12)..(12): Citrullinated Arginine
SGLKSLQTVSGRQQL

SEQ ID NO: 3 - protein, 23 aa, Artificial Sequence
  Description of Artificial Sequence Synthetic peptide
  MOD_RES (4)..(4): Citrullinated Arginine
NRARPDAEKKEMKTITGKTFSSR

SEQ ID NO: 4 - protein, 15 aa, Artificial Sequence
  Description of Artificial Sequence Synthetic peptide
  MOD_RES (3)..(3): Citrullinated Arginine
VKRQFMNKSLSGPGQ

SEQ ID NO: 5 - protein, 33 aa, Artificial Sequence
  Description of Artificial Sequence Synthetic polypeptide
  MOD_RES (19)..(19): Citrullinated Arginine
  MOD_RES (25)..(25): Citrullinated Arginine
YVLEQASSHIGNSTQATVRDAVVGRPSMDKKAQ

SEQ ID NO: 6 - protein, 33 aa, Artificial Sequence; identical to SEQ ID NO: 5
  Description of Artificial Sequence Synthetic polypeptide
  MOD_RES (19)..(19): Citrullinated Arginine

SEQ ID NO: 7 - protein, 18 aa, Artificial Sequence
  Description of Artificial Sequence Synthetic peptide
  MOD_RES (6)..(6): Citrullinated Arginine
LAENGRLSNTQGVVSAFS

SEQ ID NO: 8 - protein, 20 aa, Artificial Sequence
  Description of Artificial Sequence Synthetic peptide
  MOD_RES (6)..(6): Citrullinated Arginine
LAENGRLSNTQGVVSAFSTM

SEQ ID NO: 9 - protein, 22 aa, Artificial Sequence
  Description of Artificial Sequence Synthetic peptide
  MOD_RES (10)..(10): Citrullinated Arginine
IAQQMMPEVRGALFGANANRKF

SEQ ID NO: 10 - protein, 23 aa, Artificial Sequence
  Description of Artificial Sequence Synthetic peptide
  MOD_RES (7)..(7): Citrullinated Arginine
LLKNGERIEKVEHSDLSFSKDWS

SEQ ID NO: 11 - protein, 17 aa, Artificial Sequence
  Description of Artificial Sequence Synthetic peptide
  MOD_RES (3)..(3): Citrullinated Arginine
MARKMKDTDSEEEIREA

SEQ ID NO: 12 - protein, 17 aa, Artificial Sequence
  Description of Artificial Sequence Synthetic peptide
  MOD_RES (3)..(3): Citrullinated Arginine
MIREADIDGDGQVNYEE

SEQ ID NO: 13 - protein, 16 aa, Artificial Sequence
  Description of Artificial Sequence Synthetic peptide
  MOD_RES (2)..(2): Citrullinated Arginine
IREADIDGDGQVNYEE

SEQ ID NO: 14 - protein, 15 aa, Artificial Sequence
  Description of Artificial Sequence Synthetic peptide
  MOD_RES (2)..(2): Citrullinated Arginine
IREADIDGDGQVNYE

SEQ ID NO: 15 - protein, 17 aa, Artificial Sequence
  Description of Artificial Sequence Synthetic peptide
  MOD_RES (14)..(14): Citrullinated Arginine
DKDGNGYISAAELRHVM

SEQ ID NO: 16 - protein, 17 aa, Artificial Sequence
  Description of Artificial Sequence Synthetic peptide
  MOD_RES (12)..(12): Citrullinated Arginine
MIREADVDQDGRVNYEE

SEQ ID NO: 17 - protein, 18 aa, Artificial Sequence
  Description of Artificial Sequence Synthetic peptide
  MOD_RES (12)..(12): Citrullinated Arginine
MIREADVDQDGRVNYEEF

SEQ ID NO: 18 - protein, 16 aa, Artificial Sequence
  Description of Artificial Sequence Synthetic peptide
  MOD_RES (12)..(12): Citrullinated Arginine
MIREADVDQDGRVNYE

SEQ ID NO: 19 - protein, 15 aa, Artificial Sequence
  Description of Artificial Sequence Synthetic peptide
  MOD_RES (11)..(11): Citrullinated Arginine
IREADVDQDGRVNYE

SEQ ID NO: 20 - protein, 29 aa, Artificial Sequence
  Description of Artificial Sequence Synthetic peptide
  MOD_RES (11)..(11): Citrullinated Arginine
EAVSHTSDMHRGYADSPSKAGAAPYVQAF

SEQ ID NO: 21 - protein, 17 aa, Artificial Sequence
  Description of Artificial Sequence Synthetic peptide
  MOD_RES (7)..(7): Citrullinated Arginine
NNDNLLREGAAHAFAQY

SEQ ID NO: 22 - protein, 19 aa, Artificial Sequence
  Description of Artificial Sequence Synthetic peptide
  MOD_RES (17)..(17): Citrullinated Arginine
LPPDAEGPHGQFVSEKRVE

SEQ ID NO: 23 - protein, 20 aa, Artificial Sequence
  Description of Artificial Sequence Synthetic peptide
  MOD_RES (3)..(3): Citrullinated Arginine
IPRALAENSGVKANEVISKL

SEQ ID NO: 24 - protein, 29 aa, Artificial Sequence
  Description of Artificial Sequence Synthetic peptide
  MOD_RES (1)..(1): Citrullinated Arginine
RVDQIIMAKPAGGPKPPSGKKDWDDDQND

SEQ ID NO: 25 - protein, 25 aa, Artificial Sequence
  Description of Artificial Sequence Synthetic peptide
  MOD_RES (19)..(19): Citrullinated Arginine
NPDGPLGAAAGAAGGSWGRPGPPPA

SEQ ID NO: 26 - protein, 15 aa, Artificial Sequence
  Description of Artificial Sequence Synthetic peptide
  MOD_RES (4)..(4): Citrullinated Arginine
VTRRLGAGARAAPRR

SEQ ID NO: 27 - protein, 14 aa, Artificial Sequence
  Description of Artificial Sequence Synthetic peptide
  MOD_RES (11)..(11): Citrullinated Arginine
KLEQSEAQLGRGSF

SEQ ID NO: 28 - protein, 18 aa, Artificial Sequence
  Description of Artificial Sequence Synthetic peptide
  MOD_RES (12)..(12): Citrullinated Arginine
FNKPDIDAWELRKGINTL

SEQ ID NO: 29 - protein, 31 aa, Artificial Sequence
  Description of Artificial Sequence Synthetic polypeptide
  MOD_RES (5)..(5): Citrullinated Arginine
FVHLRVFQSLPHENKPLTLSNYQTNKAKHDE

SEQ ID NO: 30 - protein, 27 aa, Artificial Sequence
  Description of Artificial Sequence Synthetic peptide
  MOD_RES (1)..(1): Citrullinated Arginine
RVFQSLPHENKPLTLSNYQTNKAKHDE

SEQ ID NO: 31 - protein, 21 aa, Artificial Sequence
  Description of Artificial Sequence Synthetic peptide
  MOD_RES (10)..(10): Citrullinated Arginine
EVQALDDTERGSGGFGSTGKN

SEQ ID NO: 32 - protein, 18 aa, Artificial Sequence
  Description of Artificial Sequence Synthetic peptide
  MOD_RES (5)..(5): Citrullinated Arginine
INANRADAEEEAATRIPA

SEQ ID NO: 33 - protein, 20 aa, Artificial Sequence
  Description of Artificial Sequence Synthetic peptide
  MOD_RES (2)..(2): Citrullinated Arginine
LRIRSKKNEIMVAPDKDYFL

SEQ ID NO: 34 - protein, 16 aa, Artificial Sequence
  Description of Artificial Sequence Synthetic peptide
  MOD_RES (5)..(5): Citrullinated Arginine
WKVTRKDGNASGTTLL

SEQ ID NO: 35 - protein, 15 aa, Artificial Sequence
  Description of Artificial Sequence Synthetic peptide
  MOD_RES (5)..(5): Citrullinated Arginine
WKVTRKDGNASGTTL

SEQ ID NO: 36 - protein, 17 aa, Artificial Sequence
  Description of Artificial Sequence Synthetic peptide
  MOD_RES (5)..(5): Citrullinated Arginine
WKVTRKDGNASGTTLLE

SEQ ID NO: 37 - protein, 17 aa, Artificial Sequence
  Description of Artificial Sequence Synthetic peptide
  MOD_RES (11)..(11): Citrullinated Arginine
YVTIIDAPGHRDFIKNM

SEQ ID NO: 38 - protein, 33 aa, Artificial Sequence
  Description of Artificial Sequence Synthetic polypeptide
  MOD_RES (1)..(1): Citrullinated Arginine
RQTVAVGVIKAVDKKAAGAGKVTKSAQKAQKAK

SEQ ID NO: 39 - protein, 20 aa, Artificial Sequence
  Description of Artificial Sequence Synthetic peptide
  MOD_RES (8)..(8): Citrullinated Arginine
YVSNEELRGSTPEAAAQVVQ

SEQ ID NO: 40 - protein, 23 aa, Artificial Sequence
  Description of Artificial Sequence Synthetic peptide
  MOD_RES (8)..(8): Citrullinated Arginine
YVSNEELRGSTPEAAAQVVQWVS

SEQ ID NO: 41 - protein, 19 aa, Artificial Sequence
  Description of Artificial Sequence Synthetic peptide
  MOD_RES (9)..(9): Citrullinated Arginine
FKSPDDPSRYISPDQLADL

SEQ ID NO: 42 - protein, 21 aa, Artificial Sequence
  Description of Artificial Sequence Synthetic peptide
  MOD_RES (8)..(8): Citrullinated Arginine
FTSKGLFRAAVPSGASTGIYE

SEQ ID NO: 43 - protein, 15 aa, Artificial Sequence
  Description of Artificial Sequence Synthetic peptide
  MOD_RES (7)..(7): Citrullinated Arginine
GVMVSHRSGETEDTF

SEQ ID NO: 44 - protein, 22 aa, Artificial Sequence
  Description of Artificial Sequence Synthetic peptide
  MOD_RES (14)..(14): Citrullinated Arginine
LAQANGWGVMVSHRSGETEDTF

SEQ ID NO: 45 - protein, 19 aa, Artificial Sequence
  Description of Artificial Sequence Synthetic peptide
  MOD_RES (10)..(10): Citrullinated Arginine
AVEKGVPLYRHIADLAGNS

SEQ ID NO: 46 - protein, 26 aa, Artificial Sequence
  Description of Artificial Sequence Synthetic peptide
  MOD_RES (2)..(2): Citrullinated Arginine
YRQQAAYYAQTSPQGMPQHPPAPQGQ

SEQ ID NO: 47 - protein, 21 aa, Artificial Sequence
  Description of Artificial Sequence Synthetic peptide
  MOD_RES (21)..(21): Citrullinated Arginine
TVHAITATQKTVDGPSGKLWR

SEQ ID NO: 48 - protein, 17 aa, Artificial Sequence
  Description of Artificial Sequence Synthetic peptide
  MOD_RES (13)..(13): Citrullinated Arginine
ISWYDNEFGYSNRVVDL

SEQ ID NO: 49 - protein, 16 aa, Artificial Sequence
  Description of Artificial Sequence Synthetic peptide
  MOD_RES (13)..(13): Citrullinated Arginine
ISWYDNEFGYSNRVVD

SEQ ID NO: 50 - protein, 14 aa, Artificial Sequence
  Description of Artificial Sequence Synthetic peptide
  MOD_RES (11)..(11): Citrullinated Arginine
WYDNEFGYSNRVVD

SEQ ID NO: 51 - protein, 26 aa, Artificial Sequence
  Description of Artificial Sequence Synthetic peptide
  MOD_RES (19)..(19): Citrullinated Arginine
GGGNFRGGGRGGFGRGGGRGGFNKGQ

SEQ ID NO: 52 - protein, 19 aa, Artificial Sequence
  Description of Artificial Sequence Synthetic peptide
  MOD_RES (18)..(18): Citrullinated Arginine
SLTGLLLLQAVSWASGARP

SEQ ID NO: 53 - protein, 19 aa, Artificial Sequence
  Description of Artificial Sequence Synthetic peptide
  MOD_RES (6)..(6): Citrullinated Arginine
ELEAERSTNIAAAASEPHS

SEQ ID NO: 54 - protein, 19 aa, Artificial Sequence
  Description of Artificial Sequence Synthetic peptide
  MOD_RES (13)..(13): Citrullinated Arginine
MDAPVSGGVGAARSGNLTF

SEQ ID NO: 55 - protein, 28 aa, Artificial Sequence
  Description of Artificial Sequence Synthetic peptide
  MOD_RES (2)..(2): Citrullinated Arginine
VRLLLPGELAKHAVSEGTKAVTKYTSSK

SEQ ID NO: 56 - protein, 22 aa, Artificial Sequence
  Description of Artificial Sequence Synthetic peptide
  MOD_RES (10)..(10): Citrullinated Arginine
YSPQQLAGKRIGVFSYGSGLAA

SEQ ID NO: 57 - protein, 24 aa, Artificial Sequence
  Description of Artificial Sequence Synthetic peptide
  MOD_RES (19)..(19): Citrullinated Arginine
PKRKVSSAEGAAKEEPKRRSARLS

SEQ ID NO: 58 - protein, 25 aa, Artificial Sequence
  Description of Artificial Sequence Synthetic peptide
  MOD_RES (5)..(5): Citrullinated Arginine
  MOD_RES (23)..(23): Citrullinated Arginine
FAKPRNQGGYGGSSSSSSYGSGRRF

SEQ ID NO: 59 - protein, 19 aa, Artificial Sequence
  Description of Artificial Sequence Synthetic peptide
  MOD_RES (2)..(2): Citrullinated Arginine
SRAQLGGPEAAKSDETAAK

SEQ ID NO: 60 - protein, 24 aa, Artificial Sequence
  Description of Artificial Sequence Synthetic peptide
  MOD_RES (18)..(18): Citrullinated Arginine
GIYVKTIFAGGAAAADGRLQEGDE

SEQ ID NO: 61 - protein, 32 aa, Artificial Sequence
  Description of Artificial Sequence Synthetic polypeptide
  MOD_RES (3)..(3): Citrullinated Arginine
  MOD_RES (11)..(11): Citrullinated Arginine
QIRQFAAVLTRRRLNTRWRRLAAEQRESLKSL

SEQ ID NO: 62 - protein, 16 aa, Artificial Sequence
  Description of Artificial Sequence Synthetic peptide
  MOD_RES (9)..(9): Citrullinated Arginine
QWIPPPDKRRNSELFQ

SEQ ID NO: 63 - protein, 19 aa, Artificial Sequence
  Description of Artificial Sequence Synthetic peptide
  MOD_RES (1)..(1): Citrullinated Arginine
  MOD_RES (14)..(14): Oxidized Methionine
RGGLGGGYGGASGMGGITA

SEQ ID NO: 64 - protein, 20 aa, Artificial Sequence
  Description of Artificial Sequence Synthetic peptide
  MOD_RES (2)..(2): Citrullinated Arginine
LRTPKIVSGKDYNVTANSKL

SEQ ID NO: 65 - protein, 13 aa, Artificial Sequence
  Description of Artificial Sequence Synthetic peptide
  MOD_RES (8)..(8): Citrullinated Arginine
ALADEADRARAKA

SEQ ID NO: 66 - protein, 18 aa, Artificial Sequence
  Description of Artificial Sequence Synthetic peptide
  MOD_RES (7)..(7): Citrullinated Arginine
VNTNVPRASVPDGFLSEL

SEQ ID NO: 67 - protein, 30 aa, Artificial Sequence
  Description of Artificial Sequence Synthetic polypeptide
  MOD_RES (6)..(6): Citrullinated Arginine
LALRKRTPSAGKAMDTPKPAVSDEKNINTF

SEQ ID NO: 68 - protein, 19 aa, Artificial Sequence
  Description of Artificial Sequence Synthetic peptide
  MOD_RES (3)..(3): Citrullinated Arginine
WDRNNPEPWNKLGPNDQYK

SEQ ID NO: 69 - protein, 26 aa, Artificial Sequence
  Description of Artificial Sequence Synthetic peptide
  MOD_RES (7)..(7): Citrullinated Arginine
  MOD_RES (24)..(24): Citrullinated Arginine
NVVKTGRVMLGETNPADSKPGTIRGD

SEQ ID NO: 70 - protein, 27 aa, Artificial Sequence
  Description of Artificial Sequence Synthetic peptide
  MOD_RES (7)..(7): Citrullinated Arginine
NVVKTGRVMLGETNPADSKPGTIRGDF

SEQ ID NO: 71 - protein, 26 aa, Artificial Sequence
  Description of Artificial Sequence Synthetic peptide
  MOD_RES (7)..(7): Citrullinated Arginine
NVVKTGRVMLGETNPADSKPGTIRGD

SEQ ID NO: 72 - protein, 17 aa, Artificial Sequence
  Description of Artificial Sequence Synthetic peptide
  MOD_RES (7)..(7): Citrullinated Arginine
NVVKTGRVMLGETNPAD

SEQ ID NO: 73 - protein, 26 aa, Artificial Sequence
  Description of Artificial Sequence Synthetic peptide
  MOD_RES (11)..(11): Citrullinated Arginine
WAALENKSKQRTNPSPTNPFSSDLQK

SEQ ID NO: 74 - protein, 27 aa, Artificial Sequence
  Description of Artificial Sequence Synthetic peptide
  MOD_RES (11)..(11): Citrullinated Arginine
WAALENKSKQRTNPSPTNPFSSDLQKT

SEQ ID NO: 75 - protein, 18 aa, Artificial Sequence
  Description of Artificial Sequence Synthetic peptide
  MOD_RES (8)..(8): Citrullinated Arginine
LMPNGPMRITSGPFEPDL

SEQ ID NO: 76 - protein, 26 aa, Artificial Sequence
  Description of Artificial Sequence Synthetic peptide
  MOD_RES (22)..(22): Citrullinated Arginine
YSIQGQHTISPLDLAKLNQVARQQSH

SEQ ID NO: 77 - protein, 26 aa, Artificial Sequence
  Description of Artificial Sequence Synthetic peptide
  MOD_RES (21)..(21): Citrullinated Arginine
SIQGQHTISPLDLAKLNQVARQQSHF

SEQ ID NO: 78 - protein, 27 aa, Artificial Sequence
  Description of Artificial Sequence Synthetic peptide
  MOD_RES (9)..(9): Citrullinated Arginine
  MOD_RES (19)..(19): Citrullinated Arginine
AEKWTTVPRQHVCVESTDRQIKTIRPN

SEQ ID NO: 79 - protein, 18 aa, Artificial Sequence
  Description of Artificial Sequence Synthetic peptide
  MOD_RES (17)..(17): Citrullinated Arginine
LLVTGAVDCSLRGWDLRN

SEQ ID NO: 80 - protein, 22 aa, Artificial Sequence
  Description of Artificial Sequence Synthetic peptide
  MOD_RES (1)..(1): Citrullinated Arginine
RTKSTGGAPTFNVTVTKTDKTL

SEQ ID NO: 81 - protein, 24 aa, Artificial Sequence
  Description of Artificial Sequence Synthetic peptide
  MOD_RES (4)..(4): Citrullinated Arginine
HLGRPDGVPMPDKYSLEPVAVELK

SEQ ID NO: 82 - protein, 23 aa, Artificial Sequence
  Description of Artificial Sequence Synthetic peptide
  MOD_RES (3)..(3): Citrullinated Arginine
  MOD_RES (19)..(19): Citrullinated Arginine
LARELPAAVAPAGPASLARWTLG

SEQ ID NO: 83 - protein, 23 aa, Artificial Sequence
  Description of Artificial Sequence Synthetic peptide
  MOD_RES (19)..(19): Citrullinated Arginine
LARELPAAVAPAGPASLARWTLG

SEQ ID NO: 84 - protein, 19 aa, Artificial Sequence
  Description of Artificial Sequence Synthetic peptide
  MOD_RES (16)..(16): Citrullinated Arginine
RLVPFDHAESTYGLYRTHL

SEQ ID NO: 85 - protein, 15 aa, Artificial Sequence
  Description of Artificial Sequence Synthetic peptide
  MOD_RES (5)..(5): Citrullinated Arginine
QVVGRAGTGVDNVDL

SEQ ID NO: 86 - protein, 14 aa, Artificial Sequence
  Description of Artificial Sequence Synthetic peptide
  MOD_RES (9)..(9): Citrullinated Arginine
DVASVQQQRQEQSY

SEQ ID NO: 87 - protein, 25 aa, Artificial Sequence
  Description of Artificial Sequence Synthetic peptide
  MOD_RES (6)..(6): Citrullinated Arginine
GGDFTRHNGTGGKSIYGEKFEDENF

SEQ ID NO: 88 - protein, 24 aa, Artificial Sequence
  Description of Artificial Sequence Synthetic peptide
  MOD_RES (6)..(6): Citrullinated Arginine
GGDFTRHNGTGGKSIYGEKFEDEN

SEQ ID NO: 89 - protein, 23 aa, Artificial Sequence
  Description of Artificial Sequence Synthetic peptide
  MOD_RES (6)..(6): Citrullinated Arginine
GGDFTRHNGTGGKSIYGEKFEDE

SEQ ID NO: 90 - protein, 17 aa, Artificial Sequence
  Description of Artificial Sequence Synthetic peptide
  MOD_RES (3)..(3): Citrullinated Arginine
FTRHNGTGGKSIYGEKF

SEQ ID NO: 91 - protein, 27 aa, Artificial Sequence
  Description of Artificial Sequence Synthetic peptide
  MOD_RES (5)..(5): Citrullinated Arginine
ENLIRKHTGSGILSMANAGPNTNGSQF

SEQ ID NO: 92 - protein, 21 aa, Artificial Sequence
  Description of Artificial Sequence Synthetic peptide
  MOD_RES (3)..(3): Citrullinated Arginine
  MOD_RES (13)..(13): Oxidized Methionine
LIRKHTGSGILSMANAGPNTN

SEQ ID NO: 93 - protein, 26 aa, Artificial Sequence
  Description of Artificial Sequence Synthetic peptide
  MOD_RES (3)..(3): Citrullinated Arginine
  MOD_RES (13)..(13): Oxidized Methionine
LIRKHTGSGILSMANAGPNTNGSQFF

SEQ ID NO: 94 - protein, 25 aa, Artificial Sequence
  Description of Artificial Sequence Synthetic peptide
  MOD_RES (3)..(3): Citrullinated Arginine
  MOD_RES (13)..(13): Oxidized Methionine
LIRKHTGSGILSMANAGPNTNGSQF

SEQ ID NO: 95 - protein, 21 aa, Artificial Sequence
  Description of Artificial Sequence Synthetic peptide
  MOD_RES (3)..(3): Citrullinated Arginine
LIRKHTGSGILSMANAGPNTN

SEQ ID NO: 96 - protein, 19 aa, Artificial Sequence
  Description of Artificial Sequence Synthetic peptide
  MOD_RES (3)..(3): Citrullinated Arginine
LIRKHTGSGILSMANAGPN

SEQ ID NO: 97 - protein, 25 aa, Artificial Sequence
  Description of Artificial Sequence Synthetic peptide
  MOD_RES (3)..(3): Citrullinated Arginine
LIRKHTGSGILSMANAGPNTNGSQF

SEQ ID NO: 98 - protein, 26 aa, Artificial Sequence
  Description of Artificial Sequence Synthetic peptide
  MOD_RES (3)..(3): Citrullinated Arginine
LIRKHTGSGILSMANAGPNTNGSQFF

SEQ ID NO: 99 - protein, 29 aa, Artificial Sequence
  Description of Artificial Sequence Synthetic peptide
  MOD_RES (29)..(29): Citrullinated Arginine
LETADGESMNTEESNQGSTPSDQQQNKLR

SEQ ID NO: 100 - protein, 31 aa, Artificial Sequence
  Description of Artificial Sequence Synthetic polypeptide
  MOD_RES (29)..(29): Citrullinated Arginine
LETADGESMNTEESNQGSTPSDQQQNKLRSS

SEQ ID NO: 101 - protein, 15 aa, Artificial Sequence
  Description of Artificial Sequence Synthetic peptide
  MOD_RES (11)..(11): Citrullinated Arginine
WSDLDELKGARSLET

SEQ ID NO: 102 - protein, 25 aa, Artificial Sequence
  Description of Artificial Sequence Synthetic peptide
  MOD_RES (9)..(9): Citrullinated Arginine
IIDDKGILRQITVNDLPVGRSVDET

SEQ ID NO: 103 - protein, 30 aa, Artificial Sequence
  Description of Artificial Sequence Synthetic polypeptide
  MOD_RES (23)..(23): Citrullinated Arginine
VNTPKKQGGLGPMNIPLVSDPKRTIAQDYG

SEQ ID NO: 104 - protein, 26 aa, Artificial Sequence
  Description of Artificial Sequence Synthetic peptide
  MOD_RES (9)..(9): Citrullinated Arginine
IIDGKGVLRQITVNDLPVGRSVDEAL

SEQ ID NO: 105 - protein, 20 aa, Artificial Sequence
  Description of Artificial Sequence Synthetic peptide
  MOD_RES (1)..(2): Citrullinated Arginine
RRLSEDYGVLKTDEGIAYRG

SEQ ID NO: 106 - protein, 20 aa, Artificial Sequence
  Description of Artificial Sequence Synthetic peptide
  MOD_RES (14)..(14): Citrullinated Arginine
QFGEEDADPGAMSRPFGVAL

SEQ ID NO: 107 - protein, 18 aa, Artificial Sequence
  Description of Artificial Sequence Synthetic peptide
  MOD_RES (13)..(13): Citrullinated Arginine
GQENPQLLQQISRHQEQF

SEQ ID NO: 108 - protein, 21 aa, Artificial Sequence
  Description of Artificial Sequence Synthetic peptide
  MOD_RES (6)..(6): Citrullinated Arginine
LQQIGRENPQLLQQISQHQEH

SEQ ID NO: 109 - protein, 19 aa, Artificial Sequence
  Description of Artificial Sequence Synthetic peptide
  MOD_RES (11)..(11): Citrullinated Arginine
NVAEVDKVTGRFNGQFKTY

SEQ ID NO: 110 - protein, 34 aa, Artificial Sequence
  Description of Artificial Sequence Synthetic polypeptide
  MOD_RES (13)..(13): Citrullinated Arginine
LSKGLIKLVSKHRAQVIYTRNTKGGDAPAAGEDA

SEQ ID NO: 111 - protein, 32 aa, Artificial Sequence
  Description of Artificial Sequence Synthetic polypeptide
  MOD_RES (11)..(11): Citrullinated Arginine
KGLIKLVSKHRAQVIYTRNTKGGDAPAAGEDA

SEQ ID NO: 112 - protein, 16 aa, Artificial Sequence
  Description of Artificial Sequence Synthetic peptide
  MOD_RES (6)..(6): Citrullinated Arginine
MEDLDRNKDQEVNFQE

SEQ ID NO: 113 - protein, 14 aa, Artificial Sequence
  Description of Artificial Sequence Synthetic peptide
  MOD_RES (1)..(1): Oxidized Methionine
  MOD_RES (6)..(6): Citrullinated Arginine
MEDLDRNKDQEVNF

SEQ ID NO: 114 - protein, 30 aa, Artificial Sequence
  Description of Artificial Sequence Synthetic polypeptide
  MOD_RES (3)..(3): Citrullinated Arginine
MARLTWASHEKMHEGDEGPGHHHKPGLGEG

SEQ ID NO: 115 - protein, 32 aa, Artificial Sequence
  Description of Artificial Sequence Synthetic polypeptide
  MOD_RES (3)..(3): Citrullinated Arginine
MARLTWASHEKMHEGDEGPGHHHKPGLGEGTP

SEQ ID NO: 116 - protein, 32 aa, Artificial Sequence; identical to SEQ ID NO: 115
  Description of Artificial Sequence Synthetic polypeptide
  MOD_RES (1)..(1): Oxidized Methionine
  MOD_RES (3)..(3): Citrullinated Arginine

SEQ ID NO: 117 - protein, 16 aa, Artificial Sequence
  Description of Artificial Sequence Synthetic peptide
  MOD_RES (15)..(15): Citrullinated Arginine
PGHLQEGFGCVVTNRF

SEQ ID NO: 118 - protein, 19 aa, Artificial Sequence
  Description of Artificial Sequence Synthetic peptide
  MOD_RES (15)..(15): Citrullinated Arginine
PGHLQEGFGCVVTNRFDQL

SEQ ID NO: 119 - protein, 17 aa, Artificial Sequence
  Description of Artificial Sequence Synthetic peptide
  MOD_RES (17)..(17): Citrullinated Arginine
GLLDSHLIKEAGDAESR

SEQ ID NO: 120 - protein, 23 aa, Artificial Sequence
  Description of Artificial Sequence Synthetic peptide
  MOD_RES (2)..(2): Citrullinated Arginine
LRAQEAPGQAEPPAAAEVQGAGN

SEQ ID NO: 121 - protein, 22 aa, Artificial Sequence
  Description of Artificial Sequence Synthetic peptide
  MOD_RES (4)..(4): Citrullinated Arginine
  MOD_RES (10)..(10): Citrullinated Arginine
  MOD_RES (22)..(22): Citrullinated Arginine
GGTRGANGGRVPGNGAGLGPGR

SEQ ID NO: 122 - protein, 25 aa, Artificial Sequence
  Description of Artificial Sequence Synthetic peptide
  MOD_RES (2)..(2): Citrullinated Arginine
LRATDGKKKISTVVSSKEVNKFQMA

SEQ ID NO: 123 - protein, 14 aa, Artificial Sequence
  Description of Artificial Sequence Synthetic peptide
  MOD_RES (7)..(7): Citrullinated Arginine
MVAKEARNVTMETE

SEQ ID NO: 124 - protein, 12 aa, Artificial Sequence
  Description of Artificial Sequence Synthetic peptide
  MOD_RES (8)..(8): Citrullinated Arginine
LGGLAVARDDGL

SEQ ID NO: 125 - protein, 25 aa, Artificial Sequence
  Description of Artificial Sequence Synthetic peptide
  MOD_RES (24)..(24): Citrullinated Arginine
VIGLQMGTNRGASQAGMTGYGMPRQ

SEQ ID NO: 126 - protein, 20 aa, Artificial Sequence
  Description of Artificial Sequence Synthetic peptide
  MOD_RES (17)..(17): Citrullinated Arginine
TNRGASQAGMTGYGMPRQIL

SEQ ID NO: 127 - protein, 34 aa, Artificial Sequence
  Description of Artificial Sequence Synthetic polypeptide
  MOD_RES (17)..(17): Citrullinated Arginine
QLQEGKNVIGLQMGTNRGASQAGMTGYGMPRQIL

SEQ ID NO: 128 - protein, 27 aa, Artificial Sequence
  Description of Artificial Sequence Synthetic peptide
  MOD_RES (10)..(10): Citrullinated Arginine
  MOD_RES (17)..(17): Oxidized Methionine
VIGLQMGTNRGASQAGMTGYGMPRQIL

SEQ ID NO: 129 - protein, 15 aa, Artificial Sequence
  Description of Artificial Sequence Synthetic peptide
  MOD_RES (12)..(12): Citrullinated Arginine
FTKTHPQWYPARQSL

SEQ ID NO: 130 - protein, 19 aa, Artificial Sequence
  Description of Artificial Sequence Synthetic peptide
  MOD_RES (19)..(19): Citrullinated Arginine
HIQQNEALAAKAGLLGQPR

SEQ ID NO: 131 - protein, 20 aa, Artificial Sequence
  Description of Artificial Sequence Synthetic peptide
  MOD_RES (5)..(5): Citrullinated Arginine
IFPERIDPNPADSQKSTQVE

SEQ ID NO: 132 - protein, 11 aa, Artificial Sequence
  Description of Artificial Sequence Synthetic peptide
  MOD_RES (1)..(1): Citrullinated Arginine
RAEASQELLAQ

SEQ ID NO: 133 - protein, 18 aa, Artificial Sequence
  Description of Artificial Sequence Synthetic peptide
  MOD_RES (18)..(18): Citrullinated Arginine
WQGLIVPDNPPYDKGAFR

SEQ ID NO: 134 - protein, 18 aa, Artificial Sequence
  Description of Artificial Sequence Synthetic peptide
  MOD_RES (15)..(15): Citrullinated Arginine
KSVTEQGAELSNEERNLL

SEQ ID NO: 135 - protein, 31 aa, Artificial Sequence
  Description of Artificial Sequence Synthetic polypeptide
  MOD_RES (1)..(1): Oxidized Methionine
  MOD_RES (6)..(6): Citrullinated Arginine
MLVSNRVSWWGSTLAMLQRLKEQQFVIAGVL

SEQ ID NO: 136 - protein, 25 aa, Homo sapiens
RAAVPSGASTGIYEALELRDNDKTR

SEQ ID NO: 137 - protein, 25 aa, Artificial Sequence
  Description of Artificial Sequence Synthetic peptide
  MOD_RES (1)..(1): Citrullinated Arginine
  MOD_RES (19)..(19): Citrullinated Arginine
  MOD_RES (25)..(25): Citrullinated Arginine
RAAVPSGASTGIYEALELRDNDKTR

SEQ ID NO: 138 - protein, 22 aa, Homo sapiens
AMQEFMILPVGASSFREAMRGA

SEQ ID NO: 139 - protein, 22 aa, Artificial Sequence
  Description of Artificial Sequence Synthetic peptide
  MOD_RES (16)..(16): Citrullinated Arginine
  MOD_RES (20)..(20): Citrullinated Arginine
AMQEFMILPVGASSFREAMRGA

SEQ ID NO: 140 - protein, 23 aa, Homo sapiens
KSPDDPSRYITPDQLADLYKSFV

SEQ ID NO: 141 - protein, 23 aa, Artificial Sequence
  Description of Artificial Sequence Synthetic peptide
  MOD_RES (8)..(8): Citrullinated Arginine
KSPDDPSRYITPDQLADLYKSFV

SEQ ID NO: 142 - protein, 51 aa, Artificial Sequence
  Description of Artificial Sequence Synthetic polypeptide
  MOD_RES (47)..(47): Citrullinated Arginine
HIADLAGNSEVILPVPAFNVINGGSHAGNKLAMQEFMILPVGAANFREAMR

SEQ ID NO: 143 - protein, 19 aa, Artificial Sequence
  Description of Artificial Sequence Synthetic peptide
  MOD_RES (7)..(7): Citrullinated Arginine
SPDDPSRYLSPDQLADLYK

SEQ ID NO: 144 - protein, 26 aa, Artificial Sequence
  Description of Artificial Sequence Synthetic peptide
  MOD_RES (4)..(4): Citrullinated Arginine
  MOD_RES (26)..(26): Citrullinated Arginine
TLVRPSEHALVDNDGLYDPDCDPEGR

SEQ ID NO: 145 - protein, 18 aa, Artificial Sequence
  Description of Artificial Sequence Synthetic peptide
  MOD_RES (2)..(2): Citrullinated Arginine
  MOD_RES (18)..(18): Citrullinated Arginine
HRPTAGAFNHSDLDAELR

SEQ ID NO: 146 - protein, 17 aa, Artificial Sequence
  Description of Artificial Sequence Synthetic peptide
  MOD_RES (17)..(17): Citrullinated Arginine
FVAAVHYEQPTIQIELR

SEQ ID NO: 147 - protein, 16 aa, Artificial Sequence
  Description of Artificial Sequence Synthetic peptide
  MOD_RES (16)..(16): Citrullinated Arginine
AAGDVDIGDAAYYFER

SEQ ID NO: 148 - protein, 17 aa, Artificial Sequence
  Description of Artificial Sequence Synthetic peptide
  MOD_RES (11)..(11): Citrullinated Arginine
  MOD_RES (17)..(17): Citrullinated Arginine
DIKGESLFQGRGGLDLR

SEQ ID NO: 149 - protein, 29 aa, Artificial Sequence
  Description of Artificial Sequence Synthetic peptide
  MOD_RES (1)..(1): Citrullinated Arginine
RLTAGLIAVIVVVVVALVAGMAVLVITNR

SEQ ID NO: 150 - protein, 16 aa, Artificial Sequence
  Description of Artificial Sequence Synthetic peptide
  MOD_RES (16)..(16): Citrullinated Arginine
LTQLGTFEDHFLSLQR

SEQ ID NO: 151 - protein, 18 aa, Artificial Sequence
  Description of Artificial Sequence Synthetic peptide
  MOD_RES (18)..(18): Citrullinated Arginine
TIQEVAGYVLIALNTVER

SEQ ID NO: 152 - protein, 35 aa, Artificial Sequence
  Description of Artificial Sequence Synthetic polypeptide
  MOD_RES (10)..(10): Citrullinated Arginine
IPLENLQIIRGNMYYENSYALAVLSNYDANKTGLK

SEQ ID NO: 153 - protein, 16 aa, Artificial Sequence
  Description of Artificial Sequence Synthetic peptide
  MOD_RES (16)..(16): Citrullinated Arginine
FSNNPALCNVESIQWR

SEQ ID NO: 154 - protein, 26 aa, Artificial Sequence
  Description of Artificial Sequence Synthetic peptide
  MOD_RES (18)..(18): Citrullinated Arginine
  MOD_RES (26)..(26): Citrullinated Arginine
SPSDCCHNQCAAGCTGPRESDCLVCR

SEQ ID NO: 155 - protein, 25 aa, Artificial Sequence
  Description of Artificial Sequence Synthetic peptide
  MOD_RES (13)..(13): Citrullinated Arginine
  MOD_RES (25)..(25): Citrullinated Arginine
YSFGATCVKKCPRNYVVTDHGSCVR

SEQ ID NO: 156 - protein, 27 aa, Artificial Sequence
  Description of Artificial Sequence Synthetic peptide
  MOD_RES (27)..(27): Citrullinated Arginine
NYVVTDHGSCVRACGADSYEMEEDGVR

SEQ ID NO: 157 - protein, 31 aa, Artificial Sequence
  Description of Artificial Sequence Synthetic polypeptide
  MOD_RES (21)..(21): Citrullinated Arginine
  MOD_RES (27)..(27): Citrullinated Arginine
  MOD_RES (31)..(31): Citrullinated Arginine
ATGQVCHALCSPEGCWGPEPRDCVSCRNVSR

SEQ ID NO: 158 - protein, 14 aa, Artificial Sequence
  Description of Artificial Sequence Synthetic peptide
  MOD_RES (14)..(14): Citrullinated Arginine
CWGESSEDCQSLTR

SEQ ID NO: 159 - protein, 42 aa, Artificial Sequence
  Description of Artificial Sequence Synthetic polypeptide
  MOD_RES (42)..(42): Citrullinated Arginine
YTFGASCVTACPYNYLSTDVGSCTLVCPLHNQEVTAEDGTQR

SEQ ID NO: 160 - protein, 21 aa, Artificial Sequence
  Description of Artificial Sequence Synthetic peptide
  MOD_RES (10)..(10): Citrullinated Arginine
CEKCSKPCARVCYGLGMEHLR

SEQ ID NO: 161 - protein, 28 aa, Artificial Sequence
  Description of Artificial Sequence Synthetic peptide
  MOD_RES (11)..(11): Citrullinated Arginine
  MOD_RES (14)..(14): Citrullinated Arginine
VCYGLGMEHLREVRAVTSANIQEFAGCK

SEQ ID NO: 162 - protein, 22 aa, Artificial Sequence
  Description of Artificial Sequence Synthetic peptide
  MOD_RES (22)..(22): Citrullinated Arginine
ILHNGAYSLTLQGLGISWLGLR

SEQ ID NO: 163 - protein, 28 aa, Artificial Sequence
  Description of Artificial Sequence Synthetic peptide
  MOD_RES (28)..(28): Citrullinated Arginine
ELGSGLALIHHNTHLCFVHTVPWDQLFR

SEQ ID NO: 164 - protein, 19 aa, Artificial Sequence
  Description of Artificial Sequence Synthetic peptide
  MOD_RES (19)..(19): Citrullinated Arginine
GHCWGPGPTQCVNCSQFLR

SEQ ID NO: 165 - protein, 14 aa, Artificial Sequence
  Description of Artificial Sequence Synthetic peptide
  MOD_RES (14)..(14): Citrullinated Arginine
GDSGEQVGQVAWAR

SEQ ID NO: 166 - protein, 13 aa, Artificial Sequence
  Description of Artificial Sequence Synthetic peptide
  MOD_RES (13)..(13): Citrullinated Arginine
NAVQADEGEYECR

SEQ ID NO: 167 - protein, 21 aa, Artificial Sequence
  Description of Artificial Sequence Synthetic peptide
  MOD_RES (21)..(21): Citrullinated Arginine
SMNGQPLTCVVSHPGLLQDQR

SEQ ID NO: 168 - protein, 16 aa, Artificial Sequence
  Description of Artificial Sequence Synthetic peptide
  MOD_RES (16)..(16): Citrullinated Arginine
ITHILHVSFLAEASVR

SEQ ID NO: 169 - protein, 12 aa, Artificial Sequence
  Description of Artificial Sequence Synthetic peptide
  MOD_RES (12)..(12): Citrullinated Arginine
GLEDQNLWHIGR

SEQ ID NO: 170 - protein, 30 aa, Artificial Sequence
  Description of Artificial Sequence Synthetic polypeptide
  MOD_RES (30)..(30): Citrullinated Arginine
VDGDTLGFPPLTTEHSGIYVCHVSNEFSSR

SEQ ID NO: 171 - protein, 17 aa, Artificial Sequence
  Description of Artificial Sequence Synthetic peptide
  MOD_RES (17)..(17): Citrullinated Arginine
FAGVFHVEKNGRYSISR

SEQ ID NO: 172 - protein, 12 aa, Artificial Sequence
  Description of Artificial Sequence Synthetic peptide
  MOD_RES (4)..(4): Citrullinated Arginine
  MOD_RES (12)..(12): Citrullinated Arginine
DGTRYVQKGEYR

SEQ ID NO: 173 - protein, 24 aa, Artificial Sequence
  Description of Artificial Sequence Synthetic peptide
  MOD_RES (24)..(24): Citrullinated Arginine
TNPEDIYPSNPTDDDVSSGSSSER

SEQ ID NO: 174 - protein, 26 aa, Artificial Sequence
  Description of Artificial Sequence Synthetic peptide
  MOD_RES (5)..(5): Citrullinated Arginine
  MOD_RES (26)..(26): Citrullinated Arginine
VDGNRQIIGYVIGTQQATPGPAYSGR

SEQ ID NO: 175 - protein, 13 aa, Artificial Sequence
  Description of Artificial Sequence Synthetic peptide
  MOD_RES (13)..(13): Citrullinated Arginine
SDLVNEEATGQFR

SEQ ID NO: 176 - protein, 17 aa, Artificial Sequence
  Description of Artificial Sequence Synthetic peptide
  MOD_RES (17)..(17): Citrullinated Arginine
NDTASYKCETQNPVSAR

SEQ ID NO: 177 - protein, 26 aa, Artificial Sequence
  Description of Artificial Sequence Synthetic peptide
  MOD_RES (1)..(1): Citrullinated Arginine
  MOD_RES (26)..(26): Citrullinated Arginine
RSDSVILNVLYGPDAPTISPLNTSYR

SEQ ID NO: 178 - protein, 16 aa, Artificial Sequence
  Description of Artificial Sequence Synthetic peptide
  MOD_RES (3)..(3): Citrullinated Arginine
QDRTLTIVDTGIGMTK

SEQ ID NO: 179 - protein, 38 aa, Artificial Sequence
  Description of Artificial Sequence Synthetic polypeptide
  MOD_RES (26)..(26): Citrullinated Arginine
  MOD_RES (35)..(35): Citrullinated Arginine
VTVITKHNDDEQYAWESSAGGSFTVRTDTGEPMGRGTK

SEQ ID NO: 180 - protein, 29 aa, Artificial Sequence
  Description of Artificial Sequence Synthetic peptide
  MOD_RES (17)..(17): Citrullinated Arginine
HSQFIGYPITLFVEKERDKEVSDDEAEEK

SEQ ID NO: 181 - protein, 24 aa, Artificial Sequence
  Description of Artificial Sequence Synthetic peptide
  MOD_RES (24)..(24): Citrullinated Arginine
SLTNDWEDHLAVKHFSVEGQLEFR

SEQ ID NO: 182 - protein, 20 aa, Artificial Sequence
  Description of Artificial Sequence Synthetic peptide
  MOD_RES (1)..(1): Citrullinated Arginine
  MOD_RES (20)..(20): Citrullinated Arginine
RVFIMDNCEELIPEYLNFIR

SEQ ID NO: 183 - protein, 14 aa, Artificial Sequence
  Description of Artificial Sequence Synthetic peptide
  MOD_RES (14)..(14): Citrullinated Arginine
GVVDSEDLPLNISR

SEQ ID NO: 184 - protein, 26 aa, Artificial Sequence
  Description of Artificial Sequence Synthetic peptide
  MOD_RES (7)..(7): Citrullinated Arginine
  MOD_RES (26)..(26): Citrullinated Arginine
KLSELLRYYTSASGDEMVSLKDYCTR

SEQ ID NO: 185 - protein, 35 aa, Artificial Sequence
  Description of Artificial Sequence Synthetic polypeptide
  MOD_RES (6)..(6): Citrullinated Arginine
  MOD_RES (27)..(27): Citrullinated Arginine
VVVSNRLVTSPCCIVTSTYGWTANMERIMKAQALR

SEQ ID NO: 186 - protein, 16 aa, Artificial Sequence
  Description of Artificial Sequence Synthetic peptide
  MOD_RES (5)..(5): Citrullinated Arginine
AQALRDNSTMGYMAAK

SEQ ID NO: 187 - protein, 11 aa, Artificial Sequence
  Description of Artificial Sequence Synthetic peptide
  MOD_RES (2)..(2): Citrullinated Arginine
SRAPVNWYQEK

SEQ ID NO: 188 - protein, 60 aa, Artificial Sequence
  Description of Artificial Sequence Synthetic polypeptide
  MOD_RES (23)..(23): Citrullinated Arginine
KPSAGDDFDLGDAVVDGENDDPRPPNPPKPMPNPNPNHPSSSGSFSDADLADGVSGGEGK

SEQ ID NO: 189 - protein, 24 aa, Artificial Sequence
  Description of Artificial Sequence Synthetic peptide
  MOD_RES (14)..(14): Citrullinated Arginine
  MOD_RES (24)..(24): Citrullinated Arginine
ENAEQGEVDMESHRNANAEPAVQR

SEQ ID NO: 190 - protein, 21 aa, Artificial Sequence
  Description of Artificial Sequence Synthetic peptide
  MOD_RES (18)..(18): Citrullinated Arginine
VAGLESLGDLFPNLTVIRGWK

SEQ ID NO: 191 - protein, 19 aa, Artificial Sequence
  Description of Artificial Sequence Synthetic peptide
  MOD_RES (10)..(10): Citrullinated Arginine
  MOD_RES (19)..(19): Citrullinated Arginine
MEEVTGTKGRQSKGDINTR

SEQ ID NO: 192 - protein, 16 aa, Artificial Sequence
  Description of Artificial Sequence Synthetic peptide
  MOD_RES (11)..(11): Citrullinated Arginine
  MOD_RES (16)..(16): Citrullinated Arginine
GRQSKGDINTRNNGER

SEQ ID NO: 193 - protein, 18 aa, Artificial Sequence
  Description of Artificial Sequence Synthetic peptide
  MOD_RES (9)..(9): Citrullinated Arginine
  MOD_RES (18)..(18): Citrullinated Arginine
YGSQVEDQRECVSRQEYR

SEQ ID NO: 194 - protein, 17 aa, Artificial Sequence
  Description of Artificial Sequence Synthetic peptide
  MOD_RES (17)..(17): Citrullinated Arginine
SPLSTLTVYYSPETIGR

SEQ ID NO: 195 - protein, 13 aa, Artificial Sequence
  Description of Artificial Sequence Synthetic peptide
  MOD_RES (13)..(13): Citrullinated Arginine
YCCQVSNDVGPGR

SEQ ID NO: 196 - protein, 21 aa, Artificial Sequence
  Description of Artificial Sequence Synthetic peptide
  MOD_RES (14)..(14): Citrullinated Arginine
  MOD_RES (21)..(21): Citrullinated Arginine
LDQEVQEETQGRFRLLGDPSR

(19) United States
(12) Patent Application Publication
(10) Pub. No.: US 2024/0201191 A1
(43) Pub. Date: Jun. 20, 2024

(54) CITRULLINATED PROTEINS AS BIOMARKERS AND THERAPY TARGETS FOR CANCER

(71) Applicant: BOARD OF REGENTS, THE UNIVERSITY OF TEXAS SYSTEM, Austin, TX (US)

(72) Inventors: Samir HANASH, Houston, TX (US); Hiroyuki KATAYAMA, Houston, TX (US)

(21) Appl. No.: 18/285,205

(22) PCT Filed: Mar. 30, 2022
(86) PCT No.: PCT/US2022/071434

§ 371 Date: Sep. 29, 2023

(51) Int. Cl. G01N 33/574; G01N 33/68

(52) U.S. Cl. CPC G01N 33/57484; G01N 33/6848; G01N 2333/988; G01N 2440/18

(57) ABSTRACT

Provided herein are methods of detecting cancer, diagnosing a subject with cancer, treating a subject with cancer, and reducing the risk of or preventing cancer in a subject. The methods are based in part on the discovery of dysregulated protein citrullination by protein arginine deaminase enzymes in cancer. Also provided are citrullinated peptides and kits that are expressed at a higher level in cancer cells than in corresponding normal cells. Kits and systems relating to these methods are also provided.

### CROSS-REFERENCE TO RELATED APPLICATIONS

This application claims priority to and the benefit of U.S. Provisional Patent Application Ser. No. 63/168,164, filed Mar. 30, 2021, the contents of which is incorporated herein by this reference as if fully set forth herein.

### FIELD

The present disclosure relates generally to the field of cancer diagnosis and therapy. More particularly, it concerns the use of citrullinated proteins in the diagnosis and therapy of cancer.

### BACKGROUND

Despite great progress in the fields of cancer diagnosis and therapy, about 600,000 residents of the United States were projected to die of cancer in 2020.

Citrullination is the post-translational conversion of the amino acid arginine to citrulline. Protein citrullination is brought about by the action of protein arginine deiminase (PADI) family members. Dysregulated protein citrullination by PADI family members has been associated with autoimmune diseases. To date, no enzyme has been identified that can reverse protein citrullination. The role of protein citrullination has been best investigated in the context of rheumatoid arthritis (RA), where elevated protein citrullination, notably of keratins, filaggrin, vimentin, actin, histones, nucleophosmin, and nuclear lamin C, has been shown to elicit an autoimmune response. Autoimmunity in RA is considered to be principally facilitated through MHC class II mediated presentation of citrullinated peptides that elicit a B-cell response.

It would be desirable to have additional diagnostic, therapeutic, and prophylactic modalities against cancer.

### SUMMARY

The following presents a simplified summary of the disclosure in order to provide a basic understanding of some aspects of the disclosure. This summary is not an exhaustive overview of the disclosure. It is not intended to identify key or critical elements of the disclosure or to delineate the scope of the disclosure. Its sole purpose is to present some concepts in a simplified form as a prelude to the more detailed description that is discussed later.

In one aspect, provided is a method of detecting cancer and/or diagnosing a subject with cancer comprising using a citrullinated peptide or protein for detection of autoantibodies present in a biological sample from the subject, wherein said autoantibodies specifically bind to the citrullinated peptide or protein. In another aspect, provided is a method of detecting cancer and/or diagnosing a subject with cancer comprising detecting a citrullinated peptide or proteins in a biological sample from the subject. In another aspect, provided are methods of treating a subject diagnosed with cancer having elevated levels of a citrullinated peptide or protein or having autoantibodies against a citrullinated peptide or protein comprising administering an anti-cancer agent. In another aspect, provided is a method of reducing the risk of or preventing cancer in a subject comprising administering a composition comprising at least one citrullinated peptide or protein associated with the cancer to the subject.

In one embodiment, the present disclosure relates to a method, comprising providing a plasma sample from a patient suffering from a cancer or suspected of suffering from the cancer; incubating the plasma sample with at least one citrullinated protein selected from the group consisting of the proteins encoded by genes listed in Table 1, the proteins encoded by genes listed in Table 2, and the proteins encoded by genes listed in Table 10 under conditions sufficient for any autoantibodies against the citrullinated protein(s) that may be present in the plasma sample to bind to the citrullinated protein(s); incubating the citrullinated protein(s) and any bound autoantibodies against it/them with a detectable label, under conditions in which the detectable label will bind to the bound autoantibodies and will substantially not bind to other molecules; detecting the detectable label bound to the bound autoantibodies; classifying the patient as suffering from the cancer in response to the detected amount of the detectable label bound to the bound autoantibodies being equal to or greater than a threshold; and classifying the patient as not suffering from the cancer in response to the detected amount of the detectable label bound to the bound autoantibodies being less than the threshold. In some embodiments, wherein the patient as classified as suffering from the cancer, the method further comprises administering at least one anti-cancer agent to the patient.

In one embodiment, the present disclosure relates to a method of treating a patient, the method comprising administering at least one anti-cancer agent to the patient, wherein the patient has a level of autoantibodies against at least one citrullinated protein selected from the group consisting of the proteins encoded by genes listed in Table 1, the proteins encoded by genes listed in Table 2, and the proteins encoded by genes listed in Table 10 that is greater to or equal than a threshold.

In one embodiment, the present disclosure relates to a kit, comprising a substrate; at least one citrullinated protein selected from the group consisting of the proteins encoded by genes listed in Table 1, the proteins encoded by genes listed in Table 2, and the proteins encoded by genes listed in Table 10; and instructions for performing the method described above.

In one embodiment, the present disclosure relates to a method, comprising providing a tissue sample from a patient suffering from a cancer or suspected of suffering from the cancer; assaying the tissue sample for at least one citrullinated protein selected from the group consisting of the proteins encoded by genes listed in Table 1, the proteins encoded by genes listed in Table 2, and the proteins encoded by genes listed in Table 10; classifying the patient as suffering from the cancer in response to the tissue sample containing an amount of the citrullinated protein(s) equal to or greater than a threshold; and classifying the patient as not suffering from the cancer in response to the tissue sample containing an amount of the citrullinated protein(s) less than the threshold.

In one embodiment, the present disclosure relates to a kit, comprising a cell lysis agent; and instructions for performing the method described above.

In one embodiment, the present disclosure relates to a method, comprising providing a tumor sample from a patient suffering from a cancer; assaying the tumor sample for at least one citrullinated amino acid sequence selected from the group consisting of sequences and corresponding modifications listed in Table 2, Table 9, and/or Table 10, and sequences having at least 70% identity to the sequences listed in Table 2, Table 9, and/or Table 10 and comprising at least one arginine residue; and presenting to the immune system of the patient at least one peptide, wherein each peptide comprises at least one of the citrullinated amino acid sequences, in response to the tumor sample containing an amount of the citrullinated amino acid sequence(s) equal to or greater than a threshold.

In one embodiment, the present disclosure relates to a kit, comprising at least one peptide, wherein each peptide comprises at least one citrullinated amino acid sequence selected from the group consisting of sequences and corresponding modifications listed in Table 2, Table 9, and/or Table 10, and sequences having at least 70% identity to the sequences listed in Table 2, Table 9, and/or Table 10 and comprising at least one arginine residue; and instructions for performing the method described above.

In one embodiment, the present disclosure relates to a method, comprising providing a tumor sample from a patient suffering from a cancer; assaying the tumor sample for a citrullinated protein encoded by a gene selected from the group consisting of genes listed in Table 1 as having a plasma membrane location; and administering to the patient an anti-cancer agent targeting the citrullinated protein, in response to the tumor sample containing an amount of the citrullinated protein equal to or greater than a threshold.

In one embodiment, the present disclosure relates to a kit, comprising an anti-cancer agent targeting a citrullinated protein encoded by a gene selected from the group consisting of genes listed in Table 1 as having a plasma membrane location; and instructions for performing the method described above.

In one embodiment, the present disclosure relates to an isolated peptide comprising at least 70% sequence identity to a peptide selected from the group consisting of peptides listed in Table 2, Table 9, and Table 10.

In embodiments, the present disclosure may allow the diagnosis and/or therapy of cancer.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein. The drawings do not limit the scope of the compositions and methods, unless the written description expressly indicates that such is the case.

FIG. 1 shows PADI family gene and protein expression in various cancer types, according to aspects of this disclosure. Depicted is gene expression of PADI family members in the Curtis Breast cohort (ref. 36) for 144 normal breast tissues as well as 1,725 breast tumors stratified by hormone receptor subtype. Statistical significance was determined by Dunn's multiple comparison test and significant elevation of PADI2 was observed in breast cancer compared to normal control.

FIG. 2 shows hormone receptor specificity of citrullinome in Breast cancer cell lines, according to aspects of this disclosure. Top panel: scatter plot illustrating the correlation (Pearson correlation (95% CI)) between the total number of citrullinated mass spectra and PADI2 mRNA expression in breast cancer cell lines. Bottom panel: distribution plots illustrating the number of citrullinated proteins in whole cell lysate of breast cancer cell lines stratified by hormone receptor positivity. Statistical significance was determined using Wilcoxon rank sum test.

FIGS. 3A-3F show association between the citrullinome and tumor immune response in breast cancer, according to aspects of this disclosure. FIG. 3A shows relative mRNA and protein expression of PADI2 following siRNA-mediated knockdown of PADI2 in HCC1187 TNBC cell line. Statistical significance was determined by 2-sided student t-test. ***p<0.001, ****p<0.0001. FIG. 3B shows Immunoblots for anti-peptidylcitrulline following siRNA-mediated knockdown of PAD/2 in HCC1187 TNBC cell line. Bar plot to the right illustrates densitometry analysis of anti-peptidtylcitrulline normalized against beta-actin. FIG. 3C shows down regulation of citrullinome following siRNA-mediated knockdown of PADI2 in HCC1187 TNBC cell line. Scatter plots represent the delta in signal intensity of the TMT channels subtracted by siRNA-PADI2 treated cells to si-control; statistical significance was determined by 2-sided paired t-test of the citrullinated peptide TMT ratios. FIG. 3D shows cell surface MHC peptides identified in HCC1954 (Her2 enriched) and MDA-MB-468 (TNBC) breast cancer cell lines. Statistical significance was determined by Fisher's exact test. The putative MHC class 11 binding peptide length (12-34 amino acids) containing Arg citrullinationed peptides were searched against the NetMHC-II pan V.4.1 (refs. 27 and 28) considered as unmodified form and the binding affinity was presented in Table 2. FIG. 3E shows a heatmap depicting Spearman correlation coefficients between mRNA expression of PADI family members and immune gene signatures in TCGA all breast cancers (n=974). The broken line highlights the association between mRNA expression PADI family members and B cell gene-based signatures. PADI2 was strongly positively correlated with B-cell gene-based signatures. FIG. 3F shows TCGA-derived gene expression revealed elevated levels of PADI2 and gene signatures of B-cells in TNBC (n=115) compared to non-TNBC (Luminal A/B and Her2 enriched combined; n=859) tumors. Statistical significance was determined using 2-sided Wilcoxon rank sum test.

FIGS. 4A-4E show PADI2 mediated citrullination and B cell tumor infiltration, according to aspects of this disclosure. FIG. 4A shows representative IHC sections for PADI2, peptidylcitrulline (Citrulline), B cell markers CD19 and CD20, and the tumor marker PanCK in mammary gland and breast tumors stratified by hormone receptor subtype. (Original magnification ×200). FIG. 4B shows immuno-precipitated IgG bound citrullinome identified by mass spectrometry in plasma from breast cancer subjects. Distribution of citrullinated proteins normalized against total unique peptides (see methods) in 26 pooled plasma samples of newly diagnosed breast cancer consists of 156 patients and 12 pooled plasma samples corresponds to 113 cancer-free subjects (see Table 6). Statistical significance was determined by 2-sided student t-test. ***<0.001. The AUC performance of the same cohort. FIG. 4C shows autoantibody reactivity against citrullinated VIM in individual patient plasma from 11 stage H TNBC cases and 31 healthy controls. The 11 stage H TNBC patient plasmas were the same used for autoantibody reactivity against unmodified and citrullinated VIM by immune-blotting assay (FIG. S6). Statistical significance was determined by 2-sided Wilcoxon rank sum test. FIG. 4D shows classifier performance (AUC) of citrullinated VIM for distinguishing TNBC cases (n=11) from healthy controls (n=31). FIG. 4E shows autoantibody reactivity against citrullinated and unmodified VIM in TNBC case (red) and healthy control (blue) plasmas. Nodes and connecting lines represented matched samples. Statistical significance was determined by 2-sided paired t-test.

FIG. 5 shows correlation of PADI2 mRNA, mutational burden and gene-based signatures of B-cells in TCGA-breast cancer tumors, according to aspects of this disclosure. Gene expression data for the TCGA-breast cancer dataset was downloaded from CbioPortal 30. Scatter plots represent association between PADI2 mRNA, mutational burden and gene-based signatures of B-cells. Mutational burden was defined as the number of mutation events per case. Node color depicts breast cancer molecular subtype (blue—basal type; red—normal-like, green—hormone receptor (HR) positive; purple—HR-/HER2-receptor positive).

FIG. 6 shows confirmation of the reactivity of anti-peptidylcitrulline antibody, according to aspects of this disclosure.

FIG. 7 shows PADI2 and citrulline expression in healthy tissue and tumor adjacent normal tissues (n=6), according to aspects of this disclosure. Original magnification:×200.

FIG. 8 shows plasma IgG reactivity against recombinant unmodified and citrullinated Vimentin by immuno-blotting assay, according to aspects of this disclosure.

FIG. 9 shows ELISpot assay results for unmodified and citrullinated peptides from human ENO1, according to aspects of this disclosure. The data shown are from an ELiSpot IgG assay and show that citrullinated peptides significantly induced B cell response.

FIG. 10 shows ELISpot assay results for unmodified and citrullinated peptides from human ENO1, according to aspects of this disclosure. The data shown are from an ELISpot INFγ assay showing a higher amount of INFγ response in citrullinated peptide 1.

FIG. 11 presents a flowchart of a first method in accordance with embodiments herein.

FIG. 12 presents a flowchart of a second method in accordance with embodiments herein.

FIG. 13 presents a flowchart of a third method in accordance with embodiments herein.

FIG. 14 presents a flowchart of a fourth method in accordance with embodiments herein.

While the subject matter disclosed herein is susceptible to various modifications and alternative forms, specific embodiments thereof have been shown by way of example in the drawings and are herein described in detail. It should be understood, however, that the description herein of specific embodiments is not intended to limit the disclosure to the particular forms disclosed, but on the contrary, the intention is to cover all modifications, equivalents, and alternatives falling within the spirit and scope of the disclosure as defined by the appended claims. Moreover, the stylized depictions illustrated in the drawings are not drawn to any absolute scale.

### DETAILED DESCRIPTION

### I. Introduction

The present disclosure is based in part on the discovery by the inventors that protein arginine deaminase (PADI) family member PADI2 is highly expressed in several cancer types, including breast cancer. As detailed in the Examples herein, immunohistochemical analysis of breast tumor tissues revealed increased expression of PADI2 in tumors, along with a positive correlation between PADI2 protein expression and peptidyl-citrulline staining. PADI2 expression exhibited strong positive correlations with a B-cell immune signature and with MHC-Ul bound citrullinated peptides. Provided herein are diagnostic, therapeutic, and prophylactic compositions and methods for detecting, treating, and/or reducing the risk of or preventing cancer, based in part on neoantigens involved in the citrullinome immune response in cancer.

Dysregulated protein citrullination by PADI family members has been associated with autoimmune diseases, with recent interest in its relevance to cancer given the occurrence of autoimmunity as a manifestation of cancer (refs. 1 and 2). PADI comprises a family of enzymes that, in the presence of calcium ions, catalyze the post-translational modification of proteins via the deamination of arginine to citrulline. In total, 5 PADI family members are known, with sequence homology ranging from 70% to 95% (ref. 2). To date, no enzyme has been identified that can reverse protein citrullination. The role of protein citrullination has been best investigated in the context of rheumatoid arthritis (RA), where elevated protein citrullination, notably of keratins, filaggrin, vimentin, actin, histones, nucleophosmin, and nuclear lamin C, has been shown to elicit an autoimmune response (refs. 3 and 4). Autoimmunity in RA is considered to be principally facilitated through MHC class II mediated presentation of citrullinated peptides that elicit a B-cell response (refs. 5 and 6). There is also currently increased interest in MHC-H neoantigens as shaping tumor immunity (ref. 7).

Comprehensive assessment of the expression of PADI family members in cancer has been limited. PADI4 has been investigated largely with respect to its interactions with histone H3, ING4, p53, and HDAC2 (refs. 8-11). Moreover, the extent to which protein citrullination in tumors induces an immune response is largely unexplored. Studies aimed at interrogating antitumor immunity against citrulline-peptides of vimentin and a-enolase have demonstrated that these peptides can trigger a CD4+ T cell response (refs. 12 and 13), providing evidence that protein citrullination in tumors may be immunogenic. Given the antigenicity of citrullinated proteins, exploration of PADI family members among cancer types and their impact on citrullination and immune response has potential for the development of tumor vaccines with citrullinated antigens or for identification of citrullinated antigens as biomarkers for cancer detection or prediction of response to immunotherapy (refs. 7 and 14-18).

To date, PADI4 has been the most investigated among family members in the context of cancer. PADI4 is the only PADI member known to encompass a nuclear transport sequence to citrullinate nuclear proteins including histones (ref. 47). As demonstrated in the Examples and described herein, the protein expression of PADI family members was investigated among 196 cancer cell lines reflective of 12 common cancer types by proteomic profiling as well as by analysis of mRNA expression datasets from The Cancer Genome Atlas (TCGA) for 9,721 human tumors consisting of 32 different cancer types. It was found that with respect to overall expression levels, PADI2 is preferentially expressed at the protein level in cancer compared with other PADI family members. Expression of PADI2 in breast cancer cell lines was recapitulated in breast cancer TMA.

Mass spectrometry was used to further explore the association of PADI2 with the citrullinome of 28 breast cancer cell lines, and the findings of PADI2 expression and citrullination in 422 breast tumors were confirmed using immunohistochemistry (IHC). Citrullination is contingent on PADI expression and intracellular Ca²⁺ levels. In RA, cytosolic Ca²⁺ levels are increased compared with normal cellular concentrations (ref. 48), and in tumors, aberrant levels of Ca²⁺ channels and pumps were expressed (ref. 49) which may change the intercellular Ca²⁺ flux preferable to PADIs. PADI2-mediated citrullination was found to be elevated in breast tumor tissue compared with adjacent non-tumor tissue or normal mammary gland tissue and other organ sites, and IgG bound citrullinated proteins were shown to be elevated in the plasma of patients with newly diagnosed breast cancer compared with controls. In some embodiments, provided herein are methods of detecting, diagnosing, treating, and reducing the risk of or preventing cancer using autoantibodies against citrullinated proteins as cancer biomarkers and/or using citrullinated proteins and/or peptides as neoantigens and/or biomarkers.

PADI2-mediated citrullination was also found to be associated with a distinct tumor immunophenotype using TCGA gene expression datasets and immunohistochemical analysis of human breast cancer tumors. In contrast to limited prior studies of protein citrullination in cancer and their impact on immune response, the occurrence of anti-citrulline autoantibodies in RA driven by a B-cell response is well documented (ref. 5). As demonstrated herein, PADI2 exhibited statistically significant positive correlations with tumor infiltrating B cells indicative of similarities with autoimmune disease. MHC analysis of citrullinated peptides in breast cancer cell lines yielded peptides in the MHC-U binding sequence length, supporting a B cell-mediated immune response. Of interest is the occurrence of a substantial fraction of citrullinated proteins as derived from the nuclear compartment pointing to a similarity between breast cancer and autoimmune diseases, for which elevated levels of circulating antinuclear antibodies are diagnostic markers (refs. 50 and 51). Also demonstrated herein is evidence for circulating autoantibodies against citrullinated tumor-associated proteins in cancer. It is likely that autoantibodies to citrullinated proteins are likely to be cancer type and subtype specific. Network analysis yielded a cytokeratin complex in common among breast cancer subtypes whereas distinct features of hormone receptors-centered network were observed in Luminal A, and a MYC centered network was observed in TNBC.

Given the current interest in cancer vaccine development and in immunotherapy, the findings described herein point to the importance of citrullination for antigenicity. Interestingly, several citrullinated proteins were identified that have been targeted for vaccine development, including vimentin and a-enolase (refs. 12 and 13). The importance of citrullination of a-enolase in inducing anti-cancer immunity has been recently demonstrated (ref. 13). Provided herein are methods for reducing the risk of or preventing cancer using citrullinated peptides (e.g., in cancer vaccine development) to induce cancer immunity.

Various illustrative embodiments of the disclosure are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related, regulatory, and business-related constraints, which will vary from one implementation to another. Moreover, it will be appreciated that such a development effort might be complex and time-consuming but would nevertheless be a routine undertaking for those of ordinary skill in the art having the benefit of this disclosure. The description is to be read from the perspective of one of ordinary skill in the art; therefore, information well known to the skilled artisan is not necessarily included.

The present subject matter will now be described with reference to the attached figures. Various structures, systems, and devices are schematically depicted in the drawings for purposes of explanation only and so as to not obscure the present disclosure with details that are well known to those skilled in the art. Nevertheless, the attached drawings are included to describe and explain illustrative examples of the present disclosure.

### II. Terminology

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

As used herein the specification, “a” or “an” may mean one or more. As used herein in the claim(s), when used in conjunction with the word “comprising,” the words “a” or “an” may mean one or more than one.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et al. “Adding amino acids to the genetic repertoire,” 9(6): 548-54 (2005)); and all references cited therein. Beta and gamma amino acids are known in the art and are also contemplated herein as unnatural amino acids. Unless otherwise indicated, a particular amino acid sequence also implicitly encompasses conservatively modified variants thereof as well as the sequence explicitly indicated.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

The term “identity” or “substantial identity,” as used in the context of a polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “autoantibody”, as used herein, refers to an antibody that specifically binds to an endogenous molecule in a subject that produces said autoantibody. The level of such antibody is typically elevated compared to the average of any other antibodies binding specifically to such an endogenous molecule. The endogenous molecule may be an autoantigen. An autoantigen is defined as a peptide, protein, or protein complex (and sometimes DNA or RNA) that is recognized by the immune system (e.g., through autoantibodies) of a subject suffering from a specific disease or disorder. These antigens should not be, under normal conditions, the target of the immune system, but T cells instead attack cells expressing the autoantigens.

The term “cancer” refers to a disease characterized by the uncontrolled growth of aberrant cells. The term includes all known cancers and neoplastic conditions, whether characterized as malignant, soft tissue, or solid, and cancers of all stages and grades including pre- and post-metastatic cancers, as well as recurrent cancer. Examples of different types of cancer include, but are not limited to, digestive and gastrointestinal cancers such as gastric cancer (e.g., stomach cancer), colorectal cancer, gastrointestinal stromal tumors, gastrointestinal carcinoid tumors, colon cancer, rectal cancer, anal cancer, bile duct cancer, small intestine cancer, esophageal cancer, breast cancer, lung cancer (e.g., non-small cell lung cancer), gallbladder cancer, liver cancer, pancreatic cancer, appendix cancer, prostate cancer, ovarian cancer, cervical cancer, uterine cancer, renal cancer, cancer of the central nervous system, skin cancer (e.g., melanoma), lymphomas, gliomas, choriocarcinomas, head and neck cancers, osteogenic sarcomas, and blood cancers.

The term “suffering from cancer,” as used herein in relation to a subject, indicates that a cancer is detectable in the subject's body using any diagnostic technique presently known or to be discovered. “Suffering” does not require the subject to be in pain from or have any naturally-perceptible symptoms of cancer.

The term “suspected of suffering from the cancer,” as used herein in relation to a subject, indicates that the subject has one or more symptoms that, in the judgment of a physician, may indicate that the subject suffers from cancer.

A “biological sample,” as used herein, generally refers to a bodily tissue or fluid obtained from a human, preferably a mammalian subject. Exemplary subjects include, but are not limited to humans, non-human primates such as monkeys, dogs, cats, mice, rats, cows, horses, camels, goats, and sheep. In some embodiments, the subject is a human. Non-limiting examples of biological samples include blood, blood fractions or blood products (e.g., serum, plasma, platelets, red blood cells, peripheral blood mononuclear cells and the like), sputum or saliva, stool, urine, other biological fluids (e.g., lymph, prostatic fluid, gastric fluid, intestinal fluid, renal fluid, lung fluid, cerebrospinal fluid, and the like). Additionally, solid tissues, for example, tissue biopsies (e.g., tumor tissue) may be used. A biological sample may be processed prior to use in a detection assay including dilution, addition of buffer or preservative, concentration, purification, or partial purification.

### III. Methods and Kits

In one aspect, provided herein are methods of detecting cancer, diagnosing a subject with cancer, treating a subject with cancer, and/or reducing the risk of or preventing cancer in a subject. In some embodiments, the methods comprise detecting a citrullinated peptide or an autoantibody that specifically binds to a citrullinated peptide. In some embodiments, detecting an autoantibody that specifically binds to a citrullinated peptide comprises use of a citrullinated protein or peptide (e.g., as described herein). In some embodiments, the citrullinated peptide is a sequence corresponding to a portion of a protein that is known to be expressed in cancer cells. In some embodiments, the citrullinated peptide is a sequence corresponding to a portion of a protein that is highly expressed by cancer cells. In some embodiments, the protein that is highly expressed in cancer cells is not expressed or minimally expressed by corresponding normal (i.e., non-cancerous) tissues. In some embodiments, the citrullinated peptide is a sequence corresponding to a portion of a protein that is known to be expressed on the cell surface of cancer cells. In some embodiments, the citrullinated peptide is a sequence corresponding to a portion of a protein that has been targeted in cancer immunotherapy (e.g., any of the peptides listed in Table 10). In some embodiments, the citrullinated peptide is a sequence corresponding to a portion of a protein encoded by any of the genes listed in Table 1, any of the genes listed in Table 2, and/or any of the genes listed in Table 10. In some embodiments, the citrullinated peptide has at least 70% identity to any of the sequences listed in Table 2, Table 9, and/or Table 10. In some embodiments, the citrullinated peptide is a

In some embodiments of the methods provided herein, the subject is suspected of having cancer or at elevated risk of having cancer. In some embodiments, the subject is displaying symptoms of having cancer. In some embodiments, the subject has a family history of cancer. In some embodiments, the subject has one or more genetic factors (e.g., mutations) that are associated with an increased risk of having or developing cancer. In some embodiments, the subject was previously treated for cancer. In some embodiments, the subject is in remission. In some embodiments, the subject has an elevated level of any of the biomarkers described herein.

### A. Detection of Autoantibodies

The methods of detecting cancer and/or diagnosing a subject with cancer provided herein can comprise various techniques using a citrullinated peptide for detection of autoantibodies (e.g., autoantibodies that specifically bind to citrullinated peptides or proteins, also referred to herein as citrullinated protein-specific autoantibodies). As described above and demonstrated in the Examples herein, the presence of autoantibodies in a subject that bind to citrullinated proteins may indicate that the subject has cancer. For each of the citrullinated peptides described herein, the entire peptide can used in the provided methods, a fragment of the peptide may be used, a variant of the peptide may be used, or a combination of two or more of the full length peptide, a fragment, or a variant thereof as described in this disclosure may be used. For example, citrullinated peptides can be used in an immunoassay to detect citrullinated protein-specific autoantibodies in a biological sample from a subject. Citrullinated peptides used in an immunoassay can be in a cell lysate (such as, for example, a whole cell lysate or a cell fraction), or purified citrullinated peptides or fragments thereof can be used provided at least one antigenic site recognized by citrullinated protein-specific autoantibodies remains available for binding.

Generally, the biological sample is assessed for the presence of citrullinated protein-specific autoantibodies by contacting the biological sample with a citrullinated peptide or fragment or variant thereof. In some embodiments, the citrullinated peptide or fragment thereof is present in a solid tissue such as a tissue section. For example, a tissue sample comprising citrullinated peptides or fragments may be used, which may be in the form of a tissue section fixed on a carrier, for example a glass slide for microscopic analysis. For example, the solid tissue can be a tumor tissue or a tissue sample comprising tumor tissue and adjacent normal tissue. In some embodiments, the citrullinated peptide or fragment thereof is present in a sample from a mammal. Tissue sections used in immunohistochemistry are well known in the art and are commercially available from a number of companies (e.g., Asterand, Inc. (Detroit, Michigan); Euroimmun (Morris Plains, New Jersey); and Imgenex (San Diego, California)). In other embodiments, the citrullinated peptide or fragment thereof is in a cell lysate, blood, serum, cerebrospinal fluid (CSF), or urine.

In some embodiments, a liquid sample comprising citrullinated protein-specific autoantibodies from a subject may be used to practice the methods provided herein. Exemplary liquid samples include cell lysate, blood, serum, cerebrospinal fluid (CSF), and urine. A step of contacting a liquid sample comprising citrullinated protein-specific autoantibodies with a citrullinated peptide or fragment or variant thereof may be carried out by incubating an immobilized form of said peptide in the presence of the liquid sample under conditions that are compatible with the formation of a complex comprising said peptides and said citrullinated protein-specific autoantibodies. Optionally, one or more washing steps may be contemplated.

In some embodiments, the citrullinated peptide or fragment thereof is an isolated, purified citrullinated peptide or fragment thereof as discussed below. In some embodiments, the citrullinated peptide or fragment thereof is in a phage display or eukaryotic cell display library. In some embodiments, the citrullinated peptide or fragments thereof is heterologously-expressed on the surface of a cell.

In some embodiments, the biological sample is contacted with a citrullinated peptide or fragment thereof and a secondary antibody. As is well known in the art, the secondary antibody is an antibody raised against the IgG of the animal species in which the primary antibody originated. Secondary antibodies bind to the primary antibody to assist in detection, sorting and purification of target antigens to which a specific primary antibody is first bound. The secondary antibody must have specificity both for the antibody species as well as the isotype of the primary antibody being used. If a citrullinated protein-specific autoantibodies is present in the biological sample, under appropriate conditions, a complex is formed between the citrullinated peptide or fragment thereof, the citrullinated protein-specific autoantibody in the biological sample, and the secondary antibody.

A complex comprising the citrullinated protein-specific autoantibodies and citrullinated peptides or fragments may be detected using a variety of methods known to the person skilled in the art, for example immunofluorescence microscopy or spectroscopy, luminescence, NMR spectroscopy, immunodiffusion, radioactivity, chemical crosslinking, surface plasmon resonance, native gel electrophoresis, or enzymatic activity. Depending on the nature of the sample, either or both immunoassays and immunocytochemical staining techniques may be used. Enzyme-linked immunosorbent assays (ELISA), Western blot, and radioimmunoassays are methods used in the art, and can be used as described herein to detect the presence of citrullinated protein-specific autoantibodies in a biological sample. While some of these methods allow for the direct detection of the complex, in some embodiments, the second antibody is labeled such that the complex may be detected specifically owing to intrinsic properties of the label such as, for example, fluorescence, radioactivity, enzymatic activity, visibility in NMR, or MRI spectra or the like. In some embodiments, the detection method may include any of Western blot, dot blot, protein microarray, ELISA, line blot radioimmune assay, immunoprecipitation, indirect immunofluorescence microscopy, radioimmunoassay, radioimmunodiffusion, ouchterlony immunodiffusion, rocket immunoelectrophoresis, immunohistostaining, complement fixation assay, FACS, and protein chip, but is not limited thereto. Methods and compositions are described herein that can be used for detecting, by immunohistochemistry, the presence of citrullinated protein-specific autoantibodies in a biological sample. Immunohistochemical methods are well known in the art, and non-limiting exemplary methods are described in U.S. Pat. Nos. 5,073,504; 5,225,325; and 6,855,552. See also Dabbs, Diagnostic Immunohistochemistry, 2^{nd} Ed., 2006, Churchill Livingstone; and Chu & Weiss, Modern Immunohistochemistry, 2009, Cambridge University Press. It would be understood by those skilled in the art that immunohistochemistry routinely includes steps that are not necessarily discussed herein in detail such as washing the tissue samples to remove unbound secondary antibodies and the parallel staining experiments with proper controls. Exemplary detection methods are described in the Examples of this disclosure. While particular protocols are described below, variations of these assays are routine and known in the art.

In some instances, the secondary antibody is conjugated to a detectable label. Detectable labels are well known in the art and include, without limitation, a fluorescent label, an enzymatic label, a radioactive label, a luminescent label, or an affinity tag such as biotin or streptavidin. Exemplary fluorescent dyes include water-soluble rhodamine dyes, fluoresceins, 2′,7′-dichlorofluoresceins, fluorescein isothiocyanate (FITC), DyLight™ 488, phycoerythrin (PE), propidium iodide (PI), PerCP, PE-Alexa Fluor® 700, Cy5, allophycocyanin, Cy7, benzoxanthene dyes, and energy transfer dyes, as disclosed in the following references: Handbook of Molecular Probes and Research Reagents, 8^{th} ed. (2002), Molecular Probes, Eugene, OR; U.S. Pat. Nos. 6,191,278, 6,372,907, 6,096,723, 5,945,526, 4,997,928, and 4,318,846; and Lee et al., 1997, Nucleic Acids Research 25:2816-2822. Exemplary enzymatic labels include but are not limited to alkaline phosphatase (AP) and horseradish peroxidase (HP)). Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Suitable radioactive labels include, for example, ³²P, ³³P, ¹⁴C, ¹²⁵I, ¹³¹I, ³⁵S, and ³H. In some instances, the detectable label can be a heterologous polypeptide such as an antigenic tag such as, for example, FLAG, polyhistidine, hemagglutinin (HA), glutathione-S-transferase (GST), or maltose-binding protein (MBP)) for use in purifying the citrullinated peptide or antigenic fragments or variants thereof. In some instances, the detectable label can be a heterologous polypeptide that is useful as diagnostic or detectable marker such as, for example, luciferase, a fluorescent protein (such as a green fluorescent protein (GFP)), or chloramphenicol acetyl transferase (CAT). Another labeling technique which may result in greater sensitivity is the coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

In some embodiments, the method comprises contacting a citrullinated peptide or fragment or variant thereof with a biological sample from a subject and a secondary antibody having a suitable label thereon under conditions in which a complex is formed between the citrullinated peptide or antigenic fragment or variant thereof, a corresponding citrullinated protein-specific autoantibody in the biological sample, if present, and the secondary antibody, and detecting the complex formed, if formed, by detecting the label of the secondary antibody, wherein the presence of the secondary antibody is indicative of the presence of a citrullinated protein-specific autoantibody in the biological sample, and wherein the absence of the secondary antibody is indicative of the absence of a citrullinated protein-specific autoantibody in the biological sample. In some instances, the secondary antibody is detectably-labeled. Immobilization of the citrullinated peptide or antigenic fragment or variant thereof on a solid carrier (also referred to herein as a substrate) can facilitate the method of citrullinated protein-specific autoantibody detection as discussed below.

In some instances, the method comprises contacting a citrullinated peptide or antigenic fragment or variant thereof having a suitable label thereon with a biological sample from a subject, and immunoprecipitating any complex formed between the citrullinated peptide or antigenic fragment or variant thereof and a corresponding citrullinated protein-specific autoantibody in the biological sample, and monitoring for said label on any of said complexes, wherein the presence of said label is indicative of the presence of a citrullinated protein-specific autoantibody in the biological sample and the absence of said label is indicative of the absence of a citrullinated protein-specific autoantibody in the biological sample.

In some instances, the method comprises a combination of immunoprecipitation and Western blot analysis to detect the presence of a citrullinated protein-specific autoantibody in a biological sample from a subject. For example, the method may comprise contacting a citrullinated peptide or fragment or variant thereof with a biological sample from a subject under conditions in which a complex is formed between the citrullinated peptide or fragment or variant thereof and a corresponding citrullinated protein-specific autoantibody in the biological sample, if present; immunoprecipitating any complex formed between the citrullinated peptide or antigenic fragment or variant thereof and a corresponding citrullinated protein-specific autoantibody in the biological sample to produce an immunoprecipitate comprising any such complex formed; separating components of the immunoprecipitate from each other (e.g., by electrophoresis), said components comprising the citrullinated peptide or antigenic fragment or variant thereof and a corresponding citrullinated protein-specific autoantibody in the biological sample, if present; and contacting the components of the immunoprecipitate with a secondary antibody having a suitable label thereon that specifically binds to a constant region of the citrullinated protein-specific autoantibody, if present; and detecting the complex formed, if formed, by detecting the label of the secondary antibody, wherein the presence of the secondary antibody is indicative of the presence of a citrullinated protein-specific autoantibody in the biological sample, and wherein the absence of the secondary antibody is indicative of the absence of a citrullinated protein-specific autoantibody in the biological sample. For example, immunoprecipitation assay may be performed to detect the presence of citrullinated protein-specific autoantibodies in a subject by contacting recombinant citrullinated proteins with a biological sample from the subject, such as serum. Exemplary labels include any of the detectable labels described in this disclosure including, for example, fluorescent dyes and radioactive labels.

In some embodiments, an isolated, purified citrullinated peptide or antigenic fragment or variant thereof may be used in the provided methods. Protein expression and purification methods are well known in the art. However, the teachings of the present invention may not only be carried out using peptides, in particular any citrullinated peptides having the exact amino acid sequence and modifications listed in Table 2, Table 9, and/or Table 10 herein, but also using fragments or variants of such peptides. Thus, modified citrullinated peptides and antigenic fragments or variants thereof are also contemplated, such as those in which one or more amino acid residues are substituted or modified (such as with glutaraldehyde).

An “isolated” or “purified” polypeptide, or portion thereof, is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the citrullinated peptide or antigenic portion thereof is recombinantly produced, optimally culture medium represents less than about 30%, 20%, 10%, 5%, or 1% (by dry weight) of chemical precursors or non-protein-of-interest chemicals.

The term “fragment” with regard to a citrullinated protein or peptide refers to an amino acid residue sequence of a portion of the full-length protein or peptide, encompassing, for example, an amino acid residue sequence that is truncated at one or both termini by one or more amino acids. The citrullinated peptide fragment retains its antigenicity such that it is bound specifically under appropriate binding conditions by a citrullinated protein-specific autoantibody that would bind specifically to the corresponding full-length citrullinated protein or peptide under appropriate binding conditions. An antigenic portion of the citrullinated protein or peptide can be a polypeptide that is, for example, 10, 25, 50, 100, 150, 200, 250 or more amino acid residues in length of the full length citrullinated protein or peptide. Alternatively or in addition, such peptide sequence may comprise one or more internal deletions of one or more amino acid residues. Thereby the residual length of the fragment equals or exceeds the length of one or more continuous or conformational epitopes, e.g., 6, 7, 8, 9, 10, 11, 12, 13, 14, 15, 16, 17, 18, 19, 20, 21, 22, 23, 24, 25, 26, 27, 28, 29, 30, 21, 32, 33, 34, 35, 36, 37, 38, 39, 40, 41, 42, 43, 44, 45, 46, 47, 48, 49, 50, 55, 60, 65, 70, 75, 80, 85, 90, 95, 100, or more amino acid residues.

The person of skill in the art is familiar with guidelines used to design peptides having sufficient immunogenicity such as, for example, those described in Jackson, D. C., et al., Vaccine 18(3-4): 355-361 (1999) and Black, M., et al., Expert Rev. Vaccines, 9(2): 157-173 (2010). Briefly, it is desirable that the peptide meets as many as possible of the following requirements: (a) it has a high degree of hydrophilicity, (b) it comprises one or more residues selected from the group comprising aspartate, proline, tyrosine, and phenylalanine, (c) is has, for higher specificity, no or little homology with other known peptides or polypeptides, (d) it is sufficiently soluble, (e) it comprises no glycosylation or phosphorylation sites unless required for specific reasons, and (f) it contains at least one arginine residue that can be citrullinated (e.g., by PADI family enzymes). Alternatively, bioinformatics approaches may be followed such as, for example, those described by Moreau, V., et al., BMC Bioinformatics 2008, 9:71 (2008). Such biologically active portions can be prepared by recombinant techniques and evaluated for pesticidal activity.

The term “variant” of a citrullinated protein or peptide, or fragments thereof, refers to a polypeptide comprising an amino acid residue sequence that is at least 70, 75, 80, 85, 90, 92, 94, 95, 96, 97, 98 or 99% identical to the normal sequence of the citrullinated protein, peptide, or fragment thereof. Within the context of this disclosure, a variant of a citrullinated protein or peptide, or a fragment thereof, retains its antigenicity such that it is bound specifically under appropriate conditions by a citrullinated protein-specific autoantibody that would specifically bind to the corresponding full length citrullinated protein or peptide under appropriate conditions. In some instances, variants are modified at amino acid residues other than those essential for the biological activity, for example the ability of an antigen to bind specifically to a citrullinated protein-specific antibody, such as a citrullinated protein-specific autoantibody. In some instances, one or more such essential amino acid residues may optionally be replaced in a conservative manner or additional amino acid residues may be inserted such that the biological activity (i.e. antigenicity) of the variant polypeptide is preserved.

Such variants of citrullinated proteins or peptides and fragments thereof may be prepared, for example, by introducing deletions, insertions or substitutions in nucleic acid sequences encoding them, or by chemical synthesis or modification. Moreover, variants of citrullinated proteins or peptides and fragments thereof may also be generated by fusion with other known polypeptides or variants thereof and encompass active portions or domains, preferably having a sequence identity of at least 70, 75, 80, 85, 90, 92, 94, 95, 96, 97, 98 or 99%/o when aligned with the active portion of the reference sequence, wherein the term “active portion”, as used herein, refers to an amino acid sequence, which is less than the full length amino acid sequence or, in the case of a nucleic acid sequence, codes for less than the full length amino acid sequence, respectively, but retains at least some of the biological activity. For example, an active portion an antigenic polypeptide retains the ability to bind to an antibody or autoantibody and, preferably, when administered to mammals, causes an immune response to occur.

The one or more citrullinated proteins or peptides and fragments and variants thereof may be provided in any form and at any degree of purification, from tissues or cells comprising said polypeptides in an endogenous form, such as cells overexpressing the polypeptide and crude or enriched lysates of such cells, to purified and/or isolated polypeptides that are essentially pure. In embodiments, the one or more citrullinated proteins or peptides or antigenic fragments or variants thereof have a native configuration, wherein the term “native configuration”, as used herein, refers to a folded polypeptide, such as a folded polypeptide purified from tissues or cells, such as mammalian cells or tissues or from non-recombinant tissues or cells. In another embodiment, the one or more citrullinated proteins or peptides or antigenic fragments or variants thereof are recombinant proteins, wherein the term “recombinant”, as used herein, refers to a polypeptide produced using genetic engineering approaches at any stage of the production process, for example by fusing a nucleic acid encoding the polypeptide to a strong promoter for overexpression in cells or tissues or by engineering the sequence of the polypeptide itself. In another embodiment, the one or more citrullinated proteins or peptides or antigenic fragments or variants thereof are synthetic (chemically synthesized). Such techniques are well known in the art.

In some instances, the one or more citrullinated peptides or antigenic fragments or variants thereof can be denatured such as by heating, freezing or ultraviolet ray, or chemical treatments such as a surfactant or a denaturant. For example, such a denatured form may be prepared by treating them with sodium dodecyl sulfate (SDS) or dithiothreitol (DTT). Citrullinated peptides or antigenic fragments or variants thereof that are included in a kit or a panel as described herein can be provided within a cell, in a solution in which they are soluble, or the citrullinated peptides or fragments or variants thereof can be provided in a lyophilized form.

In some embodiments, the one or more citrullinated peptides or antigenic fragments or variants thereof can be immobilized on a solid carrier insoluble in an aqueous solution, such as via a covalent bond, electrostatic interactions, encapsulation or entrapment, for example by denaturing a globular polypeptide in a gel, or via hydrophobic interactions such as via one or more covalent bonds. Various suitable carriers, for example paper, metal, silicon or glass surfaces, microfluidic channels, membranes, beads such as magnetic beads, column chromatography media, biochips, polyacrylamide gels and the like have been described in the literature, for example in Kim, D., Herr, A. E. (2013), Protein immobilization techniques for microfluidic assays, Biomicrofluidics 7(4), 041501. This way, the immobilized molecule, together with the insoluble carrier, may be separated from an aqueous solution in a straightforward manner, for example by filtration, centrifugation or decanting. An immobilized molecule may be immobilized in a reversible or irreversible manner. For example, the immobilization is reversible if the molecule interacts with the carrier via ionic interactions that can be masked by addition of a high concentration of salt or if the molecule is bound via a cleavable covalent bond such as a disulfide bridge which may be cleaved by addition of thiol-containing reagents. By contrast, the immobilization is irreversible if the molecule is tethered to the carrier via a covalent bond that cannot be cleaved in aqueous solution, for example a bond formed by reaction of an epoxide group and an amine group as frequently used to couple lysine side chains to affinity columns. The protein may be indirectly immobilized, for example by immobilizing an antibody or other entity having affinity to the molecule, followed by formation of a complex to the effect that the molecule-antibody complex is immobilized. Various ways to immobilize molecules are described in the literature such as, for example, in Kim and Herr (2013). In addition, various reagents and kits for immobilization reactions are commercially available such as, for example, from Pierce Biotechnology.

In some embodiments, the citrullinated peptide or fragment thereof is present in a tissue section, and the method comprises contacting a tissue section with a biological sample and a detectably-labeled secondary antibody under conditions in which a complex is formed between citrullinated peptides in the tissue section, a corresponding citrullinated protein-specific autoantibody in the biological sample, if present, and the detectably-labeled secondary antibody; and (b) identifying a pattern of complex formation in the tissue sample by detecting the detectably-labeled secondary antibody, wherein the presence of a pattern of complex formation is indicative of the presence of citrullinated protein-specific autoantibodies in the biological sample, and wherein the absence of a pattern of complex formation is indicative of the absence of citrullinated protein-specific autoantibodies in the biological sample.

In some embodiments, the three components—the tissue section, the biological sample, and the detectably-labeled secondary antibody—are combined under conditions in which a complex is formed between citrullinated peptides in the tissue section, and a corresponding citrullinated protein-specific autoantibody in the biological sample, if present, and the detectably-labeled secondary antibody. Using the detectable label and appropriate detection means, the pattern of complex formation within the tissue sections is identified. The pattern of complex formation within the tissue sections is directly related to the cellular location(s) of the antigen (e.g., an antigenic citrullinated peptide) bound by an autoantibody, when present, in the biological sample. As described herein, the presence of a particular pattern of complex formation in one or more types of tissue indicates the presence of citrullinated protein-specific autoantibodies in the biological sample.

### B. Detection of Citrullinated Peptides or Proteins

The methods of detecting cancer and/or diagnosing a subject with cancer provided herein can comprise using a protein detection and/or quantification method for detection of citrullinated peptides or proteins (i.e., rather than detecting autoantibodies that bind to citrullinated proteins). As described above and demonstrated in the Examples herein, the presence of citrullinated proteins or peptides in a subject (e.g., in a biological sample from the subject) may indicate that the subject has cancer. Methods of detecting and quantifying proteins are known to those of skill in the art and include, but are not limited to, mass spectrometry, immunoassays (e.g., ELISA), Western blots, fluorescence microscopy, and immunohistochemistry.

In some embodiments, the citrullinated peptide that is detected and/or quantified is a sequence corresponding to a portion of a protein that is known to be expressed in cancer cells. In some embodiments, the citrullinated peptide is a sequence corresponding to a portion of a protein that is highly expressed by cancer cells. In some embodiments, the protein that is highly expressed in cancer cells is not expressed or minimally expressed by corresponding normal (i.e., non-cancerous) tissues. In some embodiments, the citrullinated peptide is a sequence corresponding to a portion of a protein that is known to be expressed on the cell surface of cancer cells. In some embodiments, the citrullinated peptide is a sequence corresponding to a portion of a protein that has been targeted in cancer immunotherapy (e.g., any of the peptides listed in Table 10). In some embodiments, the citrullinated peptide is a sequence corresponding to a portion of a protein encoded by any of the genes listed in Table 1, any of the genes listed in Table 2, and/or any of the genes listed in Table 10. In some embodiments, the citrullinated peptide has at least 70% identity to any of the sequences listed in Table 2, Table 9, and/or Table 10.

### C. Combination Methods and Assay Results

In some instances, more than one of the detection methods described above may be used in a complementary manner for more reliable results. In some embodiments, other immunoassays can be performed either in alternative to or before and/or after the immunohistochemistry methods. For example, a Western blot may be performed using, for example, a panel of known antigens associated with autoantibodies, the panel including a citrullinated peptide or antigenic fragment or variants thereof, the results of which may warrant further evaluation using, for example, the immunohistochemistry methods described herein. In another example, an immunohistochemistry method as described herein may be performed, followed by a Western blot in order to, for example, further confirm the specific antigens, including the citrullinated peptide, recognized by the autoantibodies in the biological sample.

Any data demonstrating the presence or absence of a citrullinated protein-specific autoantibody and the citrullinated peptide or antigenic fragment or variant thereof may be correlated with reference data. For example, detection of a citrullinated protein-specific autoantibody may indicate that the subject who provided the sample analyzed has cancer (e.g., a specific type or subtype of cancer). If the subject has been previously diagnosed, the amount of citrullinated protein-specific autoantibodies detected at the time of prior diagnosis and in the present time may be correlated to find out about the progression of the disease and/or the success of a treatment. For example, if the amount of citrullinated protein-specific autoantibodies is found to increase, it may be concluded that the disease is progressing and/or that any treatment attempted is unsuccessful.

### D. Treatment

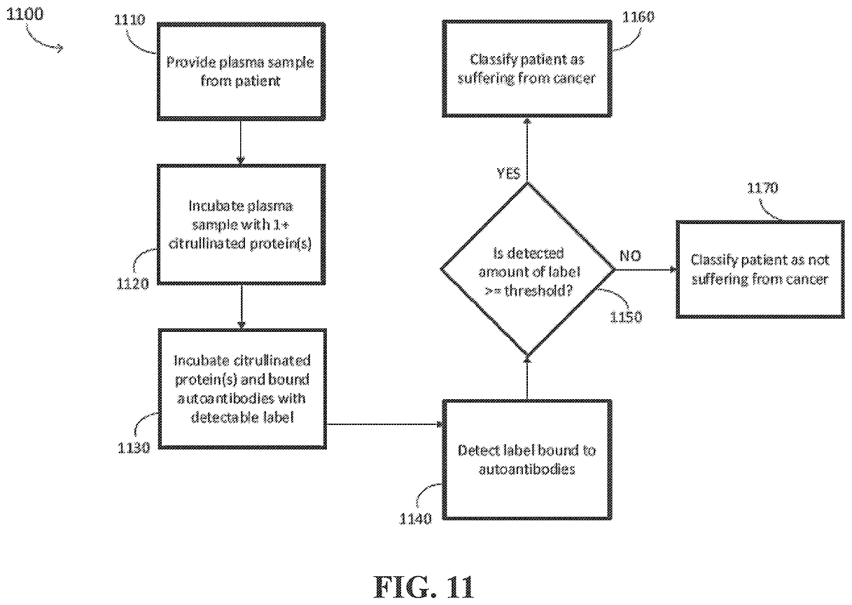

Also provided herein are methods of treating cancer. In some embodiments, the methods comprise targeting anti-cancer agents to a citrullinated protein or peptide. In some embodiments, the citrullinated protein or peptide is expressed by a cancer cell. Some embodiments of the diagnostic methods provided herein (e.g., the illustrative embodiments depicted in FIGS. 11-14 and described below) may further comprise steps of administering to a subject (e.g., a subject having or found to have elevated levels of a biomarker as described herein) one or more anti-cancer agents. In some embodiments, provided herein are methods of treating a subject with cancer, wherein the subject has elevated levels of one or more biomarkers as described herein.

In some embodiments, anti-cancer agents that may be used comprise immunotherapeutic agents. As used throughout, immunotherapy is a therapy that uses the subject's own immune system to treat cancer in the subject. Examples of cancer immunotherapy include, but are not limited to, monoclonal antibodies, chimeric antigen receptors, antibody-drug conjugates, bispecific antibodies (e.g., bispecific T engagers, bispecific NK cell engagers, etc.), immune checkpoint inhibitors, cancer vaccines, cytokines and interferons. In some embodiments, the immunotherapy comprises administering to the subject an antibody that specifically binds to a citrullinated protein or peptide (e.g., as described herein). In some embodiments, the antibody is a human antibody, chimeric antibody, humanized antibody, an F(ab)′2, an Fab, an Fv, a single domain antibody, a bispecific antibody, a helix-stabilized antibody, a single-chain antibody molecule, a disulfide stabilized antibody, or a domain antibody.

In some embodiments, the immunotherapy comprises administering to the subject a T cell, natural killer cell, or macrophage comprising a chimeric antigen receptor that specifically binds to a citrullinated protein or peptide (e.g., as described herein). Chimeric antigen receptors (CARs, also known as chimeric T cell receptors) are designed to be expressed in host effector cells, e.g., T cells, NK cells, or macrophages, and to induce an immune response against a specific target antigen (e.g., a citrullinated peptide as described herein) and cells expressing that antigen (e.g., cancer cells expressing citrullinated proteins or peptides as described herein). Adoptive T cell immunotherapy, in which a patient's own T lymphocytes are engineered to express CARs, has shown great promise in treating hematological malignancies. CARs can be engineered and used as described, for example, in Sadelain et al., 2013, Cancer Discov. 3:388-398. A CAR typically comprises an extracellular target-binding module, a transmembrane (TM) domain, and an intracellular signaling domain (ICD). The CAR domains can be joined via flexible hinge and/or spacer regions. The extracellular target-binding module generally comprises an antibody or antigen binding fragment thereof (e.g., an antibody or antigen binding fragment thereof that specifically binds to citrullinated proteins or peptides as described herein).

Immunotherapy can comprise the administration of monoclonal antibodies. Monoclonal antibodies are designed to attach to a specific target. The monoclonal antibodies used to treat liver cancer affect a tumor's ability to form new blood vessels, also known as angiogenesis. These therapeutics are often referred to angiogenesis inhibitors and include: Bevacizumab (Avastin), which can be used in conjunction with the immunotherapy drug atezolizumab (Tecentriq); Ramucirumab (Cyramza).

In some embodiments of the methods for treating a subject with cancer provided herein, an additional cancer therapy is administered to the subject. In some embodiments, the additional therapy comprises surgical treatment for the cancer. For example, the patient may receive surgical resection (removal of the tumor with surgery). Small tumors may also be treated with other types of treatment such as ablation or radiation. Ablation is treatment that destroys tumors without removing them. These techniques can be used in patients with a few small tumors and when surgery is not a good option. They are less likely to cure the cancer than surgery, but they can still be very helpful for some people. Ablation is best used for tumors no larger than 3 cm across. For slightly larger tumors (1 to 2 inches, or 3 to 5 cm across), it may be used along with embolization. Because ablation often destroys some of the normal tissue around the tumor, it might not be a good choice for treating tumors near major blood vessels, the diaphragm, or major bile ducts. In some embodiments, the ablation is radiofrequency ablation (RFA). In some embodiments, the ablation is microwave ablation (MWA). In some embodiments, the ablation is cryoablation (cryotherapy). In some embodiments, the ablation is ethanol (alcohol) ablation, e.g., percutaneous ethanol injection (PEI).

In some embodiments, a patient with cancer is also treated using radiation therapy. Radiation therapy uses high-energy rays, or particles to destroy cancer cells. Radiation can be helpful, e.g., in treating cancer that cannot be removed by surgery, cancer that cannot be treated with ablation or did not respond well to such treatment; cancer that has spread to areas such as the brain or bones; patients experiencing severe pain due to large cancers; and patients having a tumor thrombus.

In some embodiments, a patient with cancer is also treated using drug therapy, e.g., targeted drug therapy or chemotherapy. Targeted drugs work differently from standard chemotherapy drugs and include, e.g., kinase inhibitors; Sorafenib (Nexavar), lenvatinib (Lenvima), Regorafenib (Stivarga), and cabozantinib (Cabometyx). Common chemotherapy drugs for treating cancer include, for example: Gemcitabine (Gemzar); Oxaliplatin (Eloxatin); Cisplatin; Doxorubicin (pegylated liposomal doxorubicin); 5-fluorouracil (5-FU); Capecitabine (Xeloda); Mitoxantrone (Novantrone), or combinations thereof. Chemotherapy can be regional when drugs are inserted into an artery that leads to the part of the body with the tumor. thereby focusing the chemotherapy on the cancer cells in that area of the body and reducing side effects by limiting the amount of drug reaching the rest of the body. For example, hepatic artery infusion (HAI), or chemo given directly into the hepatic artery, is an example of a regional chemotherapy that can be used for liver cancer.

In some embodiments, the additional therapy is a monoclonal antibody therapy, checkpoint inhibitor therapy, oncolytic virus therapy, or thermal therapy.

Thus, in one aspect, provided herein is a method for treating cancer in a subject comprising administering an effective amount of an immunotherapeutic agent targeting a citrullinated peptide as described herein and at least one additional cancer treatment.

Also provided herein are methods of reducing the risk of or preventing cancer in a subject. In some embodiments, the methods comprise inducing a protective immune response in the subject by administering a composition comprising at least one citrullinated peptide or protein associated with the cancer to the subject. As used herein, a “protective immune response” refers to an immune response induced after administration of a vaccine composition to a subject where, upon exposure to the source of the antigenic component of the vaccine (e.g., a citrullinated peptide or cell expressing the antigen), the clinical symptoms elicited by the source are diminished. In some embodiments, inducing a protective immune response comprises administering a vaccine to the subject, wherein the vaccine comprises at least one citrullinated peptide or protein associated with the cancer.

In some embodiments, the vaccine comprises a cancer neoantigen. In some embodiments, the cancer neoantigen comprises a citrullinated peptide or protein. In some embodiments, the citrullinated peptide or protein is immunogenic (i.e., able to induce a protective immune response). In some embodiments, the citrullinated peptide is a sequence corresponding to a portion of a protein that is known to be expressed in cancer cells. In some embodiments, the citrullinated peptide is a sequence corresponding to a portion of a protein that is highly expressed by cancer cells. In some embodiments, the protein that is highly expressed in cancer cells is not expressed or minimally expressed by corresponding normal (i.e., non-cancerous) tissues. In some embodiments, the citrullinated peptide is a sequence corresponding to a portion of a protein that is known to be expressed on the cell surface of cancer cells. In some embodiments, the citrullinated peptide is a sequence corresponding to a portion of a protein that has been targeted in cancer immunotherapy (e.g., any of the peptides listed in Table 10). In some embodiments, the citrullinated peptide is a sequence corresponding to a portion of a protein encoded by any of the genes listed in Table 1, any of the genes listed in Table 2, and/or any of the genes listed in Table 10. In some embodiments, the citrullinated peptide has at least 70% identity to any of the sequences listed in Table 2, Table 9, and/or Table 10. In some embodiments, the citrullinated peptide is optimized for vaccination according to known methods for peptide vaccine selection and preparation (e.g., as described in Example 7 herein).

Cancer neoantigens (e.g., citrullinated peptides or proteins as described herein) can be recognized by tumor-infiltrating cytotoxic CD8⁺ T cells, and increased immune cell infiltration and the related cytotoxicity signatures have been observed in tumors with a higher neoantigen load. Accordingly, neoantigen presentation and load have been positively correlated with prognosis in patients with a variety of cancers and with benefit from immune-checkpoint inhibitors (ICIs) in patients with melanoma, non-small-cell lung cancer (NSCLC) or colorectal cancer with mismatch-repair deficiency. Together, these studies highlight the potential therapeutic benefit of developing immunotherapies that specifically “train” the immune system to target neoantigens.

Vaccines have traditionally been used for the prevention of infectious diseases, however, the ability of such agents to elicit and amplify antigen-specific immune responses has long been recognized as a potentially valuable tool for the treatment of cancer. Early therapeutic vaccination strategies focused on self-antigens abnormally expressed or overexpressed in tumors, termed tumor-associated antigens (TAAs), were largely unsuccessful in generating clinically effective antitumor immune responses, probably owing to the TAA-specific T cells being subject to central and/or peripheral tolerance. Such TAAs can also be expressed to some extent in nonmalignant tissues, which raises the risk of vaccine-induced autoimmune toxicities. Thus, these early studies highlighted the lack of tumor specificity and poor immunogenicity as fundamental issues to overcome in developing cancer vaccines.

Vaccines predicated on neoantigens rather than traditionally used TAAs have several advantages. First, neoantigens are exclusively expressed by tumor cells and can, therefore, elicit truly tumor-specific T cell responses, thereby preventing “off-target” damage to nonmalignant tissues. Second, the neoantigens described herein are de novo epitopes derived from citrullination of proteins expressed in cancer cells, which presents the possibility to circumvent T cell central tolerance of self-epitopes and thus induce immune responses to tumors. Personalized neoantigen-based vaccines therefore afford the opportunity to boost tumor-specific immune responses and add an additional tool to the immunotherapy toolbox. Furthermore, the potential of these vaccine-boosted neoantigen-specific T cell responses to persist and provide post-treatment immunological memory presents the possibility of long-term protection against disease recurrence.

### E. Exemplary Embodiments

Exemplary embodiments are described below with reference to FIGS. 11-14.

FIG. 11 presents a flowchart of an exemplary method of detecting cancer and/or diagnosing a subject with cancer 1100 in accordance with embodiments of the present disclosure. The method 1100 comprises providing (at 1110) a plasma sample from a patient (i.e., a subject) suffering from a cancer or suspected of suffering from the cancer.

In some embodiments, the patient is a human. In embodiments, the present method may be performed in a veterinary context. That is, the patient may be any non-human mammal suffering from a cancer. The non-human mammal may be a research animal, a pet, livestock, a working animal, a racing animal (e.g., a horse, a dog, a camel, etc.), an animal at stud (e.g., a bull, a retired racing stallion, etc.) or any other non-human mammal for which it is desired to treat its cancer. For convenience, the present disclosure will typically refer to human patients. However, the person of ordinary skill in the art having the benefit of the present disclosure will readily be able to adapt the teachings of the present disclosure to a veterinary context.

The cancer may be any cancer known to the person of ordinary skill in the art having the benefit of the present disclosure. Generally, the present disclosure considers cancers characterized by increased protein citrullination to be of greatest interest. Examples of such cancers include, but are not limited to, breast cancers, lung cancers, skin cancers, endometrial cancers, ovarian cancers, and colorectal cancers.

The plasma sample may be taken from the patient's bloodstream and purified of intact cells by any appropriate technique known to the person of ordinary skill in the art having the benefit of the present disclosure.

The first method 1100 also comprises incubating (at 1120) the plasma sample with at least one citrullinated protein selected from the group consisting of the proteins encoded by genes listed in Table 1, the proteins encoded by genes listed in Table 2, and the proteins encoded by genes listed in Table 10, under conditions sufficient for any autoantibodies against the citrullinated protein(s) that may be present in the plasma sample, to bind to the citrullinated protein(s).

Though not to be bound by theory, as demonstrated in the Examples herein, cancers characterized by increased protein citrullination may elicit autoantibodies, i.e., the patient's immune system may develop antibodies against proteins commonly citrullinated in the patient's cancer cells, and such antibodies may circulate in the patient's plasma. On the other hand, if the patient is suspected of suffering from the cancer but is not actually suffering from the cancer, the patient's plasma will have very few, if any, autoantibodies against citrullination of proteins commonly citrullinated in the cancer.

In one embodiment, the citrullinated protein(s) is/are selected from the group consisting of citrullinated vimentin and citrullinated α-enolase.

Incubating (at 1120) may be performed by any appropriate technique. Such techniques can be performed as a routine matter by the person of ordinary skill in the art having the benefit of the present disclosure. The conditions sufficient for autoantibodies in the plasma sample, if any, to bind to the citrullinated protein(s) can be established by the person of ordinary skill in the art as a routine matter. In embodiments, the conditions may comprise immobilizing the citrullinated protein(s) on a substrate, such as, e.g., the wells of a 96-well plate, among others.

The first method 1100 also comprises incubating (at 1130) the citrullinated protein(s) and any bound autoantibodies against it/them with a detectable label, under conditions wherein the detectable label will bind to the bound autoantibodies and will substantially not bind to other molecules.

Detectable labels generally comprise (i) a binding moiety that will bind to a molecule of interest, in this case, the autoantibodies (if any) bound to the citrullinated protein(s) and (ii) a moiety that produces a detectable signal, either constitutive or induced. Examples of binding moieties that can be used in the detectable label of the first method 1100 include non-human antibodies against human immunoglobulins, among others. Examples of moieties producing detectable signals include moieties comprising radioisotopes, moieties comprising chromophores, moieties comprising fluorophores, enzymatic active sites catalyzing the conversion of a molecule to a product having a different detectable signal than the molecule, among others. Detectable labels are generally known to the person of ordinary skill in the art and need not be described in detail. Conditions wherein the detectable label will bind to the bound autoantibodies and will substantially not bind to other molecules can be implemented as a routine matter.

In one embodiment, the detectable label is an antibody against the autoantibody, wherein the antibody comprises a fluorescent moiety.

The first method 1100 also comprises detecting (at 1140) the label bound to the bound autoantibodies. The precise implementation of detecting (at 1140) will depend on the label's moiety producing a detectable signal, and is a routine matter for the person of ordinary skill in the art.

In one embodiment, the incubating (at 1120), incubating (at 1130), and detecting (at 1140) may be performed as part of an enzyme-linked immunosorbent assay (ELISA), which is a well-known and long-established assay.

The intensity of the signal detected (at 1140) can be processed to provide an amount of the label bound to the bound autoantibodies. The detected amount of the label is then considered in a decision block (1150). If the amount of the label detected is greater than or equal to a threshold, a relatively large number of autoantibodies is present, from which it can be inferred that the patient has a relatively large amount of the citrullinated protein(s) in his/her body, which is a marker of a cancer characterized by increased protein citrullination. Hence, flow passes from the decision block 1150 along the YES path to classifying (at 1160) the patient as suffering from the cancer, in response to the detected amount of the label bound to the bound autoantibodies being equal to or greater than a threshold.

On the other hand, if the amount of the label detected is less than the threshold, few if any autoantibodies are present, from which it can be inferred that the patient has a relatively low amount of the citrullinated protein(s) in his/her body, which indicates that the patient is free of a cancer characterized by increased protein citrullination. In this circumstance, flow passes from the decision block 1150 to classifying (at 1170) the patient as not suffering from the cancer, in response to the detected amount of the label bound to the bound autoantibodies being less than the threshold.

After classifying (at 1160), the patient may receive a cancer treatment, such as surgical resection, chemotherapy, monoclonal antibody therapy, checkpoint inhibitor therapy, oncolytic virus therapy, thermal therapy (e.g., RFA, microwave ablation, and/or cryotherapy), radiotherapy, and two or more thereof, among others. In the other circumstance, after classifying (at 1170), the patient may be spared the expense and inconvenience of unnecessary cancer treatment, and/or the patient may be tested for other medical conditions, including other cancers not characterized by increased protein citrullination.

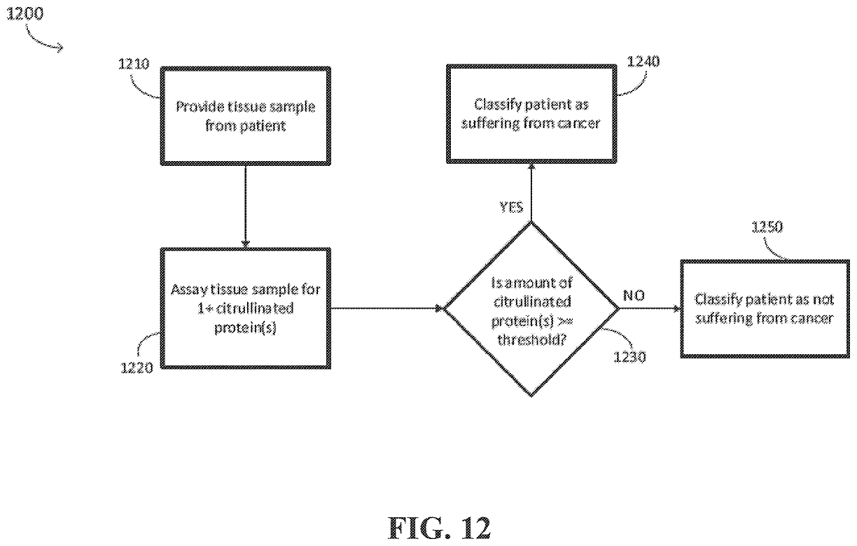

FIG. 12 presents a flowchart of a second method 1200 in accordance with embodiments herein. The second method 1200 comprises providing (at 1210) a tissue sample from a patient suffering from a cancer or suspected of suffering from the cancer. Typically, the tissue sample is taken from a tumor or a suspected tumor. This may be performed by well-known techniques that need not be described in detail.

The cancer may be any cancer known to the person of ordinary skill in the art having the benefit of the present disclosure. Generally, the present disclosure considers cancers characterized by increased protein citrullination to be of greatest interest. Examples of such cancers include, but are not limited to, breast cancers, lung cancers, skin cancers, endometrial cancers, ovarian cancers, and colorectal cancers.

The second method 1200 also comprises assaying (at 1220) the tissue sample for at least one citrullinated protein selected from the group consisting of the proteins encoded by genes listed in Table 1, the proteins encoded by genes listed in Table 2, and the proteins encoded by genes listed in Table 10. “Assaying” may involve any qualitative, semi-qualitative, or quantitative evaluation of the tissue sample's citrullinated protein(s) content. When two or more citrullinated proteins are assayed, they may be evaluated individual or in the aggregate.

Assaying (at 1220) may comprise any known techniques. In one embodiment, the assaying (at 1220) comprises lysing cells of the tissue sample, to yield a tissue sample cell lysate, and isolating a protein fraction from the tissue sample cell lysate. Citrullinated protein(s) in the isolated protein fraction may be identified and quantified as a routine matter by the person of ordinary skill in the art having the benefit of the present disclosure. In one embodiment, the assaying (at 1220) may comprise liquid chromatography-mass spectrometry (LC-MS).

In one embodiment, the citrullinated protein(s) is/are selected from the group consisting of citrullinated vimentin and citrullinated α-enolase.

The results of the assaying (at 1220) are considered at decision block 1230. If the tissue sample contains an amount of the citrullinated protein(s) equal to or greater than a threshold, flow passes to classifying (at 1240) the patient as suffering from the cancer. If the amount of the citrullinated protein(s) in the tissue sample is less than the threshold, flow instead passes to classifying (at 1250) the patient as not suffering from the cancer.

After classifying (at 1240), the patient may receive a cancer treatment, such as surgical resection, chemotherapy, monoclonal antibody therapy, checkpoint inhibitor therapy, oncolytic virus therapy, thermal therapy (e.g., RFA, microwave ablation, and/or cryotherapy), radiotherapy, a cancer vaccine comprising the citrullinated protein(s), a targeted therapy against the citrullinated proteins (e.g., CAR-T therapy), and two or more thereof, among others. In the other circumstance, after classifying (at 1250), the patient may be spared the expense and inconvenience of unnecessary cancer treatment, and/or the patient may be tested for other medical conditions, including other cancers not characterized by increased protein citrullination.

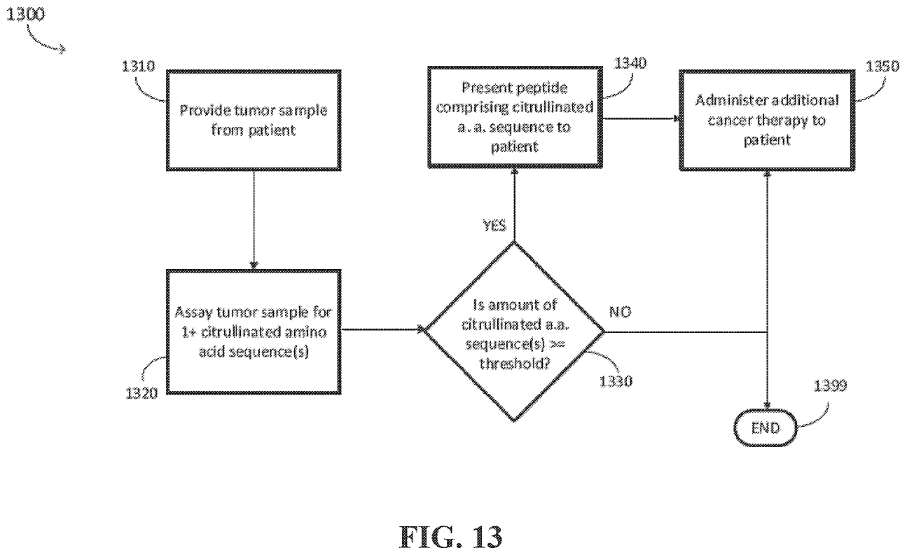

Turning to FIG. 13, a flowchart depicting a third method 1300 is shown. The third method 1300 comprises providing (at 1310) a tumor sample from a patient suffering from a cancer.

The patient, the cancer, the tumor sample, and techniques for the provision thereof have been described above and/or are well-known in the art, and need not be described further.

The third method 1300 also comprises assaying (at 1320) the tumor sample for at least one citrullinated amino acid sequence selected from the group consisting of sequences and corresponding modifications listed in Table 2, Table 9, and/or Table 10, and sequences having at least 70% identity to the sequences listed in Table 2, Table 9, and/or Table 10 and comprising at least one arginine residue. In one embodiment, the citrullinated amino acid sequences(s) is/are selected from the group consisting of GVMVSHR*SGETEDTF (SEQ ID NO:43), LAQANGWGVMVSHR*SGETEDTF (SEQ ID NO:44), and AVEKGVPLYR*HIADLAGNS (SEQ ID NO:45), wherein R* is citrulline.

Assaying (at 1320) may generally be performed as the assaying action 1220 described above with reference to FIG. 12.

The results of assaying (at 1320) are considered at decision block 1330. If the tumor sample contains an amount of (a) citrullinated amino acid sequence(s) equal to or greater than a threshold, then the third method 1300 flows to presenting (at 1340) to the immune system of the patient at least one peptide, wherein each peptide comprises at least one of the citrullinated amino acid sequences present in an amount at or above the threshold. By presenting (at 1410), the patient's immune system may develop antibodies against the citrullinated amino acid sequence, i.e., the citrullinated amino acid sequence may be considered a cancer vaccine.

The peptide(s) comprising the citrullinated amino acid sequence(s) that are presented (at 1340) may be derived from the tumor sample of the patient, may be synthesized to comprise the citrullinated amino acid sequence(s), or a combination of thereof. The peptide(s) may be provided as-is and/or may be presented by and/or encoded by an oncolytic virus.

Cancer vaccine techniques are undergoing rapid development of which the person of ordinary skill in the art having the benefit of the present disclosure will be aware.

The cancer vaccine arising from the presenting (at 1340) may be sufficient treatment for the patient's cancer. Even so, in embodiments, the third method 1300 may further comprise administering (at 1350), to the patient, an additional cancer therapy selected from the group consisting of surgical resection, chemotherapy, monoclonal antibody therapy, checkpoint inhibitor therapy, oncolytic virus therapy, thermal therapy, radiotherapy, and two or more thereof.

The additional cancer therapies referred to above are well-known and need not be described in detail. They may be implemented as a routine matter by the person of ordinary skill in the art having the benefit of the present disclosure.

Returning to the decision block 1330, if the tumor sample contains an amount of (a) citrullinated amino acid sequence(s) equal to or greater than a threshold, then flow passes out the NO node, to one of two destinations. In one embodiment, flow passes to administering (at 1350) the additional therapy/ies discussed above. Alternatively, the third method 1300 may terminate (at 1399).

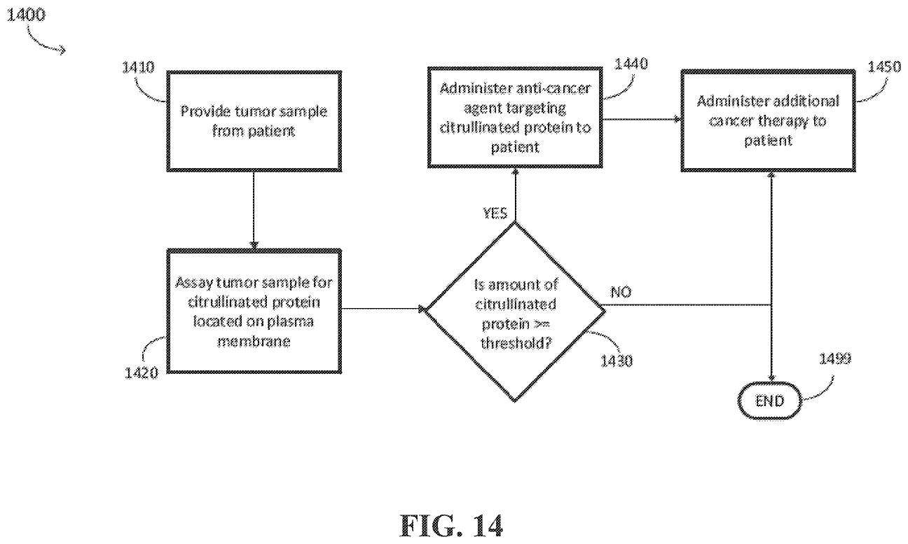

Turning to FIG. 14, a flowchart of a fourth method 1400 is presented. The fourth method 1400 comprises providing (at 1410) a tumor sample from a patient suffering from a cancer. The providing (at 1410) may be substantially as set forth above concerning previous methods, and need not be described in detail here.

The fourth method 1400 also comprises assaying (at 1420) the tumor sample for a citrullinated protein encoded by a gene selected from the group consisting of genes listed in Table 1 as having a plasma membrane location.

The assaying (at 1420) may be substantially as described above relating to other methods. By “plasma membrane location” is meant that a preponderance of the citrullinated protein in the tumor sample is exposed on the cell surface of cancer cells.

After assaying (at 1420), flow passes to decision block 1430. If the amount of citrullinated protein is greater than or equal to a threshold amount, it may be reasonably inferred that the citrullinated protein has a significant presence on the cell surface of cancer cells. Not to be bound by theory, but it is unlikely that the citrullinated protein has any significant presence on the cell surface of healthy cells. Accordingly, the citrullinated protein may provide a target for homing in an anti-cancer agent to cancer cells possessing the citrullinated protein on their cell surfaces, with little if any attack by the anti-cancer agent on healthy tissues.

Accordingly, flow in this circumstance may follow the YES path to administering (at 1440) to the patient an anti-cancer agent targeting the citrullinated protein, in response to the tumor sample containing an amount of the citrullinated protein equal to or greater than a threshold.

Similarly to the third method 1300, the fourth method 1400 may further comprise administering (at 1450), to the patient, an additional cancer therapy not targeting the citrullinated protein, as described below. The administering (at 1450) may be performed with administering (at 1440), i.e., the fourth method 1400 may provide a combination therapy; and/or the administering (at 1450) may be performed if the assay (at 1420) discovered insufficient amounts of the citrullinated protein, thereby leading flow from decision block 1430 along the NO path. Alternatively, the NO path may lead to termination of the fourth method 1400 (at 1499).

### F. Determining Patient Cancer Status

In the detection and diagnostic methods provided herein, the presence of cancer in a subject is determined by detecting levels of biomarkers, e.g., citrullinated protein-specific autoantibody biomarkers or citrullinated protein or peptide biomarkers, in a biological sample. As used herein, a “biomarker” refers to a molecule whose level in a biological sample, e.g., a blood sample such as a plasma sample, is correlated with the presence or absence of cancer (e.g., a specific type or subtype of cancer). The levels of each of the biomarkers need not be correlated with the cancer status in all subjects; rather, a correlation will exist at the population level, such that the level is sufficiently correlated within the overall population of individuals with cancer that it can be combined with the levels of other biomarkers, in any of a number of ways, as described elsewhere herein, and used to determine the cancer status. The values used for the measured level of the individual biomarkers can be determined in any of a number of ways, including direct readouts from relevant instruments or assay systems, e.g., as described below and/or using means known to those of skill in the art. In some embodiments, the readout values of the biomarkers are compared to the readout value of a reference or control, a peptide or other molecule whose level does not vary according to cancer status and whose level is measured at the same time as the biomarkers.

The term “correlating” generally refers to determining a relationship between one random variable with another. In various embodiments, correlating a given biomarker level with the presence or absence of cancer comprises determining the presence, absence or amount of at least one biomarker in a subject with the same outcome. In specific embodiments, a set of biomarker levels, absences or presences is correlated to a particular outcome, using receiver operating characteristic (ROC) curves.

In some embodiments, as demonstrated in the Examples herein, AUC values are used as a measure of the ability of a biomarker or combination of biomarkers to determine the cancer status of an individual. The “area under curve” or “AUC” refers to area under a ROC curve. AUC under a ROC curve is a measure of accuracy. An area of 1 represents a perfect test, whereas an area of 0.5 represents an insignificant test. For suitable biomarkers as described herein, the AUC may be between 0.700 and 1. For example, the AUC may be at least about 0.700, at least about 0.750, at least about 0.800, at least about 0.810, at least about 0.820, at least about 0.830, at least about 0.840, at least about 0.850, at least about 0.860, at least about 0.870, at least about 0.880, at least about 0.890, at least about 0.900, at least about 0.910, at least about 0.920, at least about 0.930, at least about 0.940, at least about 0.950, at least about 0.960, at least about 0.970, at least about 0.980, at least about 0.990, or at least about 0.995.

Additional cancer biomarkers can be assessed and identified using any standard analysis method or metric, e.g., by analyzing data from biological samples taken from subjects with or without a diagnosis of cancer, as described in more detail elsewhere herein and as illustrated, e.g., in the Examples. In some embodiments, differences in data between groups (e.g., between samples from cancer patients and samples from healthy patients without cancer) can be evalutated using two groups comparison tests, e.g., Student's T-tests, Welch's T-tests, or the Mann-Whitney U test, or multiple comparison tests, e.g., Kruskal Wallis Tests or Analysis of Variance (ANOVA) tests. In some embodiments, principal component analysis (PCA) or partial least squares discriminate analysis (PLS-DA) can be performed. Receiver operating characteristic (ROC) curves can be generated, e.g., using the pROC package in R, and the AUCs calculated with a 95% confidence interval as well as sensitivity and specificity values. Different learning algorithms, including deep learning, gradient boosting machine, auto-machine learning, iterative random forest, LASSO regularization, and bionomial logistic regression analysis can be performed, e.g., for the analysis of combinations of multiple variables.

To determine the presence of cancer (i.e., the “cancer status”) in an individual (i.e., a subject or patient), the measured biomarker levels in a sample obtained from the individual are generally compared to reference levels, e.g., levels taken from a healthy individual without cancer. The reference control levels can be measured at the same time as the biomarker levels, i.e., using the same sample, or can be a level determined based on previous measurements.

When using multiple biomarkers, it is not necessary that all of the biomarkers are elevated or depressed relative to control levels in a sample, e.g., a plasma sample, from a given subject to give rise to a determination of cancer. For example, for a given biomarker level there can be some overlap between individuals falling into different probability categories. However, collectively the combined levels for all of the biomarkers included in the assay gives rise to an AUC score that indicates a high probability of, e.g., the presence of cancer.

In some embodiments, the levels of the selected biomarkers are quantified and compared to one or more preselected or threshold levels. Threshold values can be selected that provide an ability to predict the presence or absence of cancer. Such threshold values can be established, e.g., by calculating receiver operating characteristic (ROC) curves using a first population with cancer and a second population without cancer.

In some embodiments, measuring the levels of biomarkers described herein (e.g., citrullinated protein-specific autoantibodies or citrullinated peptides) comprises the detection and quantification (e.g., semi-quantification) of the selected biomarkers in the sample. In some embodiments, the measured biomarker levels are adjusted relative to one or more standard level(s) (“normalized”). As known in the art, normalizing is done to remove technical variability inherent to a platform to give a quantity or relative quantity.

In some embodiments, the measurement of differential levels of specific biomarkers from biological samples may be accomplished using a range of technologies, reagents, and methods. These include any of the methods of measurement as described elsewhere herein.

The biomarker levels are typically normalized following detection and quantification as appropriate for the particular platform using methods routinely practiced by those of ordinary skill in the art.

Threshold or cut-off values can be adjusted to change test performance, e.g., test sensitivity and specificity. For example, the threshold for cancer may be intentionally lowered to increase the sensitivity of the test for cancer, if desired.

Determining the accuracy of detection and/or diagnosis may involve the use of accuracy measures such as sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting cancer.

It will be appreciated that for any particular biomarker, a distribution of biomarker levels for subjects with and without cancer may overlap. Under such conditions, a test does not absolutely distinguish the two populations (i.e., with or without cancer) with 100% accuracy, and the area of overlap indicates where the test cannot distinguish them. A threshold value is selected, above which the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982)).

In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more biomarkers are selected to discriminate between subjects with cancer and subjects without cancer with at least about 70%, 75%, 80%, 85%, 90%, 95% accuracy or having a C-statistic of at least about 0.70, 0.75, 0.80, 0.85, 0.90, 0.95.

The phrases “assessing the likelihood” and “determining the likelihood,” as used herein, refer to methods by which the skilled artisan can predict the presence or absence of a condition (e.g., cancer) in a patient. The skilled artisan will understand that this phrase includes within its scope an increased probability that a condition (e.g., cancer) is present or absent in a patient; that is, that a condition is more likely to be present or absent in a subject. For example, the probability that an individual identified as having a specified condition actually has the condition can be expressed as a “positive predictive value” or “PPV.” Positive predictive value can be calculated as the number of true positives divided by the sum of the true positives and false positives. PPV is determined by the characteristics of the predictive methods of the present methods as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 900, 91%, 92%, 93%, 94%, 95%, 96%, 97%, 98%, or 99%.

In other examples, the probability that an individual identified as not having a specified condition or outcome actually does not have that condition can be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined to have a significant probability of having or not having a specified condition or outcome (e.g., cancer). By “significant probability” is meant that the subject has a reasonable probability (0.6, 0.7, 0.8, 0.9 or more) of having, or not having, a specified condition or outcome.

In some embodiments, a detection of cancer can be based not solely on biomarker levels, but can also take into account clinical and/or other data about the subject, e.g., clinical data about the subject's current medical state (e.g., the presence of cancer-related symptoms), the presence of any symptoms characteristic of cancer, the medical history of the subject, the presence of one or more risk factors for HCC (e.g., family history of cancer, genetic factors including mutant alleles relevant to cancer), and/or demographic data about the subject (age, sex, etc.).

Kits and devices useful for performing the provided methods of detecting cancer and/or diagnosing a subject with cancer are described below.

### G. Measurement Systems and Reports for Detecting and Recording Biomarker Expression

In one aspect, a system, e.g., measurement system is provided. Such systems allow, e.g., the detection of biomarker levels (e.g., citrullinated protein-specific autoantibody biomarkers or citrullinated protein or peptide biomarkers) in a sample and the recording of the data resulting from the detection. The stored data can then be analyzed to determine the cancer status of a subject. Such systems can comprise, e.g., assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

Also provided is a system for detecting citrullinated protein-specific autoantibody biomarkers or citrullinated protein or peptide biomarkers in a sample, by utilizing a station for analyzing the sample (e.g., by mass spectrometry (Mass Spec or MS), liquid chromatography/mass spectrometry (LC/MS), enzyme-linked immunosorbent assay (ELISA), or immunohistochemistry (IHC)) to detect one or more (e.g., two or more) citrullinated protein-specific autoantibody biomarkers or citrullinated protein or peptide biomarkers, such as, e.g., any of the citrullinated proteins or peptides described herein (e.g., the peptides listed in Table 2, Table 9, and Table 10) or autoantibodies that specifically bind to such proteins or peptides, and the sample is a sample of a bodily fluid obtained from a subject (e.g., a blood sample such as a plasma sample) or a tissue sample obtained from a subject. In some embodiments, the system comprises a station suitable for the assay used to detect the biomarkers. For example, a mass spectrometer and/or a liquid chromatography system for MS and/or LC/MS, a plate reader for ELISA, and/or a microscope for IHC. Optionally, a station for generating a report containing information on results of the analyzing is further included.

Also provided is a method of generating a report containing information on results of the detection of citrullinated protein-specific autoantibody biomarkers or citrullinated protein or peptide biomarkers in a sample, including detecting one or more (e.g., two or more) biomarkers in the sample, and generating the report, wherein the one or more (e.g., two or more) are any of the citrullinated proteins or peptides described herein (e.g., the peptides listed in Table 2, Table 9, and Table 10) or autoantibodies that specifically bind to such proteins or peptides; the sample is a sample of a bodily fluid obtained from a subject (e.g., a blood sample such as a plasma sample) or a tissue sample obtained from the subject, and the report is useful for diagnosing cancer in the subject.

### H. Computer/Diagnostic Systems for Determining Cancer Status

Certain aspects of the methods described herein may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that uses inputted biomarker expression (and optionally other) data, and determines the cancer status of a subject.

A computer system can include desktop and laptop computers, tablets, mobile phones and other mobile devices. The system can include various elements such as a printer, keyboard, storage device(s), monitor (e.g., a display screen, such as an LED), peripherals, devices to connect a computer system to a wide area network such as the Internet, a mouse input device, scanner, a storage device(s), computer readable medium, camera, microphone, accelerometer, and the like. Any of the data mentioned herein can be output from one component to another component and can be output to the user.

In one aspect, the present disclosure provides a computer implemented method for determining the presence or absence of cancer in a patient. The computer performs steps comprising, e.g.,: receiving inputted patient data comprising values for the levels of one or more biomarkers in a biological sample from the patient; analyzing the levels of one or more biomarkers and optionally comparing them to respective reference values, optionally comparing the biomarker levels to one or more threshold values to determine cancer status; and displaying information regarding the cancer status or probability in the patient. In certain embodiments, the inputted patient data comprises values for the levels of a plurality of biomarkers in a biological sample from the patient, e.g., biomarkers comprising one or more pairs or three-way combinations of biomarkers comprising any of the citrullinated proteins or peptides described herein (e.g., the peptides listed in Table 2, Table 9, and Table 10) or autoantibodies that specifically bind to citrullinated proteins or peptides as described herein.

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the cancer status of the subject. For example, the storage component includes instructions for determining cancer status based on biomarker levels, as described herein. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### 1. Kits and Panels

Also provided herein are kits and panels that can be used for performing the methods described herein. In some embodiments, the kits and panels comprise one or more citrullinated peptides or antigenic fragments or variants thereof to which citrullinated protein-specific autoantibodies can specifically bind. As used herein, a “kit” refers to a package containing the listed materials, and instructions of any form that are provided in connection with the materials in a manner such that a clinical professional will clearly recognize that the instructions are to be associated with the materials. As used herein, “instructions” typically involve written text or graphics on or associated with packaging of materials. Instructions also can include any oral or electronic instructions provided in any manner. Written text or graphics may include a website URL or a QR code encoding a website URL, where other instructions or supplemental information may be provided in electronic form.

The peptide used in the kits and panels is preferably designed such that it is immunogenic, particularly that it binds to citrullinated protein-specific autoantibodies from subjects. In some instances, the kits include a panel as provided herein, such as a prognostic or diagnostic panel.

In certain embodiments, a kit as described herein includes one or more solubilizing agents for increasing the solubility of a peptide such as, for example, a buffer solution. The kit may further include reagents that provide a detectable signal when used in conjunction with the citrullinated peptides or fragments or variants thereof and a biological sample. In some embodiments, the kit includes a detectably-labeled secondary antibody that is able to bind to a citrullinated protein-specific autoantibody specifically binding to said one or more citrullinated peptides or fragments or variants thereof. Reagents for the detection of the secondary antibody label can also be included in the kit. The secondary antibody is detected by a method that depends on a labeling group used. Exemplary labels for secondary antibodies are described above in this disclosure.

In addition, a kit can include directions for using the citrullinated peptides or fragments or variants thereof and/or directions for practicing a method described herein; particularly, detecting citrullinated protein-specific autoantibodies in a biological sample. The concentration or amount of citrullinated protein-specific autoantibodies contained in the biological sample is indirectly measured by measuring the amount of the detectable label. The obtained measurement value may be converted to a relative or absolute concentration, amount, activity, etc. using a calibration curve or the like.

In some embodiments, a kit or a panel as provided herein includes a reference sample, such as a normal control sample. In some embodiments, a kit or a panel as provided herein includes one or more control antibody that detects an antigen that is expected to be present in a biological sample such as, for example, a biological sample from a healthy subject, or a biological sample from a subject with cancer. If such a sample is included, the obtained measurement values for such sample are compared with the results of the test sample, so that the presence or absence of cancer in the subject or biological sample can be more objectively determined.

In addition to the one or more citrullinated peptides, fragments, and/or variants, the panel can include additional polypeptides such as, for example, positive or negative controls or other antigens known to bind to autoantibodies of prognostic and/or diagnostic value, particularly those related to cancer and/or autoimmune disease.

In one aspect, provided herein is a prognostic or diagnostic device comprising a panel as described above, the panel including one or more citrullinated peptides or antigenic fragments or variants thereof. In some embodiments, such a prognostic or diagnostic panel device comprises one or more citrullinated peptides, fragments, or variants in a form as described above that allows contacting it with an aqueous solution, more preferably the liquid human sample, in a straightforward manner. In particular, the one or more citrullinated peptides, fragments, or variants may be immobilized on the surface of a carrier (also referred to as a substrate), which carrier comprises, but is not limited to glass plates or slides, biochips, microtiter plates, beads, for example magnetic beads, chromatography columns, membranes or the like. Exemplary devices include line blots, microtiter plates and biochips. In some embodiments, the device can include additional polypeptides such as, for example, positive or negative controls or other antigens known to bind to autoantibodies of prognostic and/or diagnostic value, particularly those related to cancer or autoimmune diseases as discussed above.

In some embodiments, the kits provided herein comprise a substrate; at least one citrullinated protein selected from the group consisting of the proteins encoded by genes listed in Table 1, the proteins encoded by genes listed in Table 2, and the proteins encoded by genes listed in Table 10; and instructions for performing a method, comprising instructions to provide a plasma sample from a patient suffering from a cancer or suspected of suffering from the cancer; incubate the plasma sample with the citrullinated protein(s) under conditions sufficient for any autoantibodies against the citrullinated protein(s) that may be present in the plasma sample to bind to the citrullinated protein(s); incubate the citrullinated protein(s) and any bound autoantibodies against it/them with a detectable label under conditions wherein the detectable label will bind to the bound autoantibodies and will substantially not bind to other molecules; detect the label bound to the bound autoantibodies; classify the patient as suffering from the cancer in response to the detected amount of the label bound to the bound autoantibodies being equal to or greater than a threshold; and classify the patient as not suffering from the cancer in response to the detected amount of the label bound to the bound autoantibodies being less than the threshold.

The kit may contain one or more containers, each of which can contain one or more of the materials. The kits also may contain instructions for mixing, diluting, using, or administering the materials. The kits also can include other containers with one or more solvents, surfactants, preservatives, buffers, washes, and/or diluents (e.g., normal saline (0.9% NaCl), or 5% dextrose) as well as containers for mixing, diluting, incubating, washing, etc.

The materials may be provided in any suitable form, for example, as a liquid solution or as a dried product. When the material provided is a dry product, the material may be reconstituted by the addition of solvent, which may also be provided by the kit. In embodiments where liquid forms of a material are used, the liquid form may be concentrated or ready to use.

The kit, in one embodiment, may comprise a carrier being compartmentalized to receive in close confinement one or more containers such as vials, tubes, and the like

The substrate may be as described hereinabove. The citrullinated protein(s) may also be as described hereinabove.

In addition to the substrate and the citrullinated protein(s), in embodiments, the kit may also comprise the detectable label, and any materials required to allow the label to generate a detectable signal.

The method to which the instructions relate may be as described hereinabove.

In some embodiments, the kits provided herein are useful for diagnostic methods comprising detecting citrullinated peptides and/or proteins (e.g., in a biological sample from a subject). In some embodiments, the kits comprise a cell lysis agent; and instructions for performing a method, comprising instructions to providing a tissue sample from a patient suffering from a cancer or suspected of suffering from the cancer; lyse cells of the tissue sample by exposing the tissue sample to the cell lysis agent; assay the tissue sample for at least one citrullinated protein selected from the group consisting of the proteins encoded by genes listed in Table 1, the proteins encoded by genes listed in Table 2, and the proteins encoded by genes listed in Table 10; classify the patient as suffering from the cancer, in response to the tissue sample containing an amount of the citrullinated protein(s) equal to or greater than a threshold; and classify the patient as not suffering from the cancer, in response to the tissue sample containing an amount of the citrullinated protein(s) less than the threshold.

Any cell lysis agent may be included in the kit. In one embodiment, the cell lysis agent may comprise octyl glucoside, and the instructions may further comprise instructions to prepare the octyl glucoside for use by bringing to 1% wt/vol in phosphate-buffered saline (PBS).

The kit may comprise other materials as described above or routinely included in such kits.

The method for which the kit provides instructions may be as described above.

In some embodiments, the kits provided herein comprise at least one peptide, wherein each peptide comprises at least one citrullinated amino acid sequence selected from the group consisting of sequences and corresponding modifications listed in Table 2, Table 9, and/or Table 10, and sequences having at least 70% identity to the sequences listed in Table 2, Table 9, and/or Table 10 and comprising at least one arginine residue; and instructions for performing a method, comprising instructions to: provide a tumor sample from a patient suffering from a cancer: assay the tumor sample for the citrullinated amino acid sequence(s) contained in each of the peptide(s); and present to the immune system of the patient one or more of the peptides, in response to the tumor sample containing an amount equal to or greater than a threshold of the citrullinated amino acid sequence contained in the peptide(s).

The citrullinated amino acid sequence(s) and the peptide(s) have been described above.

The method for which the instructions are provided can be as described above.

In some embodiments, the kit may further comprise a cell lysis agent, and the instructions may further comprise instructions to assay the tumor sample by lysing cells of the tumor sample, to yield a tumor sample cell lysate, and quantifying the amount of the citrullinated amino acid sequence(s) in the tumor sample cell lysate.

Alternatively or in addition, the instructions may further comprise instructions to administer, to the patient, an additional cancer therapy selected from the group consisting of surgical resection, chemotherapy, monoclonal antibody therapy, checkpoint inhibitor therapy, oncolytic virus therapy, thermal therapy, radiotherapy, and two or more thereof. In a further embodiment, wherein the additional cancer therapy is selected from the group consisting of chemotherapy, monoclonal antibody therapy, checkpoint inhibitor therapy, and oncolytic virus therapy, the kit may further comprise one or more of a chemotherapeutic agent, a monoclonal antibody, a checkpoint inhibitor, or an oncolytic virus.

In some embodiments, the kits provided herein comprise an anti-cancer agent targeting a citrullinated protein encoded by a gene selected from the group consisting of genes listed in Table 1 as having a plasma membrane location; and instructions for performing a method, comprising instructions to provide a tumor sample from a patient suffering from a cancer; assay the tumor sample for the citrullinated protein; and administer to the patient an anti-cancer agent targeting the citrullinated protein, in response to the tumor sample containing an amount of the citrullinated protein equal to or greater than a threshold.

Anti-cancer agents and citrullinated proteins have been described above.

The method for which the instructions are provided can be as described above.

In one embodiment, the kit may further comprise a cell lysis agent, and the instructions may further comprise instructions to assay the tumor sample by lysing cells of the tumor sample, to yield a tumor sample cell lysate, and quantifying the amount of the citrullinated amino acid sequence(s) in the tumor sample cell lysate.

Alternatively or in addition, the instructions may further comprise instructions to administer, to the patient, an additional cancer therapy selected from the group consisting of surgical resection, chemotherapy, monoclonal antibody therapy, checkpoint inhibitor therapy, oncolytic virus therapy, thermal therapy, radiotherapy, and two or more thereof. In a further embodiment, wherein the additional cancer therapy is selected from the group consisting of chemotherapy, monoclonal antibody therapy, checkpoint inhibitor therapy, and oncolytic virus therapy, the kit may further comprise one or more of a chemotherapeutic agent, a monoclonal antibody, a checkpoint inhibitor, or an oncolytic virus.

### IV. Polypeptides

In embodiments, the present disclosure relates to an isolated peptide comprising at least 70% sequence identity to a peptide selected from the group consisting of peptides listed in Table 2, Table 9, and Table 10.

In some embodiments, the peptide comprises at least 6 contiguous amino acids (e.g., at least 7, at least 8, at least 9, at least 10, at least 11, at least 12, at least 13, at least 14, or at least 15 contiguous amino acids) of a peptide listed in Table 2, Table 9, and/or Table 10.

In some embodiments, the peptide is 15 amino acids or fewer (e.g., 14, 13, 12, 11, 10, 9, 8, 7, 6 amino acids or fewer) in length.

In some embodiments, the peptide is around 9 amino acids in length.

In some embodiments, the peptide is around 15 amino acids in length.

In some embodiments, the peptide is 20-25 amino acids in length.

In some embodiments, the peptide is 22-24 amino acids in length.

In some embodiments, the peptide is immunogenic.

In some embodiments, the peptide is modified, i.e., one or more bonds present in the wild-type peptide is replaced with a bond to an atom or a molecule absent from the wild-type peptide.

In some embodiments, the modification comprises conjugation to a molecule. For example, the molecule may comprise an antibody, a lipid, an adjuvant, or a detection moiety.

In some embodiments, the peptide has at least 90% sequence identity to a peptide listed in Table 2, Table 9, and/or Table 10.

In some embodiments, the peptide has 1, 2 or 3 substitutions relative to a peptide listed in Table 2, Table 9, and/or Table 10.

In some embodiments, the peptide comprises 100% sequence identity to a peptide listed in Table 2, Table 9, and/or Table 10.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. Many of the following examples are further described in Katayama et al., 2021, “Protein citrullination as a source of cancer neoantigens,” Journal for ImmunoTherapy of Cancer 9:e002549, which is hereby incorporated by reference in its entirety. Reference is made to this Katayama et al. 2021 publication for illustration of certain experimental data as described in the instant disclosure.

### Example 1. Materials and Methods

### A. Mass-Spectrometry Analyses

Breast cancer cell line citrullinome analysis. Proteomic analysis was performed as previously described (refs. 19-22). For in-depth citrullinome analysis, 28 Breast cancer cell lines MCF7, MDA-MB-231, SKBR3, HCC1954, HCC1143, BT474, HCC1500, T47D, ZR75-1, HCC1937, HCC1599, HCC202, HCC1806, MDA-MB-468, HCC2218, HCC70, HCC1187, Hs578T, BT549, MCF10A, MCF12A, MDA-MB-361, HCC1395, CAMA1, HCC38, MDA-MB-436, BT20 and MDA-MB-157 were labeled with 13C6 Lys (#CNLM-2247, Cambridge isotope laboratories) in RPMI1640 containing 10% dialyzed FBS and 1% penicillin/streptomycin cocktail (Gibco). Stable Isotope Labeling by Amino acid in Cell culture (SILAC labeling) (ref. 23) was performed to discriminate FBS derived proteins from cell proteins.

For proteomic analysis of whole-cell lysates, −2×10⁷ cells were lysed in 1 mL of phosphate-buffered saline (PBS) containing octyl-glucoside (1% w/v) and protease inhibitors (complete protease inhibitor cocktail, Roche Diagnostics), followed by sonication and centrifugation at 20,000×g with collection of the supernatant and filtration through a 0.22 μm filter. Two milligrams of whole cell extract (WCE) proteins were reduced in dithio-threitol (DTT) and alkylated with acrylamide before fractionation with reversed phase-high performance liquid chromatography (RP-HPLC). A total of 84 fractions were collected at a rate of 3 fractions/min. Mobile phase A consisted of water (H₂O):acetonitrile (ACN) (95:5, v/v) with 0.1% of trifluoroacetic acid (TFA). Mobile phase B consisted of ACN:H₂O (95:5) with 0.1% of TFA. Collected fractions from HPLC were dried by lyophilization, followed by in-solution digestion with trypsin (Mass Spectrometry Grade, Thermo Fisher).

Based on the chromatogram profile, 84 fractions were pooled into 24 fractions for liquid chromatography-tandem mass spectrometry (LC-MS/MS) analysis per cell line. In total, 2,688 fractions were subjected to reversed phase LC-MS/MS (RPLC-MS/MS) using a nanoflow LC system (EASYnano HPLC system, Thermo Scientific) coupled online with LTQ Orbitrap ELITE mass spectrometer (Thermo Scientific). Separations were performed using 75 μm inner diameter (id)×360 μm outer diameter (od)×25-cm-long fused-silica capillary column (Column Technology) slurry packed with 3 sm, 100 A° pore size C18 silica-bonded stationary phase. Following injection of ˜500 ng of protein digest onto a C18 trap column (Waters, 180 μm idx20 mm), peptides were eluted using a linear gradient of 0.35% mobile phase B (0.1 formic acid in ACN) per minute for 90 min, then to 95% B for an additional 10 min, all at a constant flow rate of 300 nL/min. Eluted peptides were analyzed by LTQ Orbitrap ELITE in data-dependent acquisition mode. Each full MS scan (m/z 400-1800) was followed by 20 MS/MS scans (collision-induced dissociation (CID) normalized collision energy of 35%). Acquisition of each full mass spectrum was followed by the acquisition of MS/MS spectra for the 20 most intense +2, +3 or +4 ions within a duty cycle, dynamic exclusion was enabled to minimize redundant selection of peptides previously selected for MS/MS analysis. Parameters for MS1 were 60,000 for resolution, 1×10⁶ for automatic gain control target, and 150 ms for maximum injection time. MS/MS was done by CID fragmentation with 3×10⁴ for automatic gain control, 10 ms for maximum injection time, 35 for normalized collision energy, 2.0 m/z for isolation width, 0.25 for activation q-value, and 10 ms for activation time.

MS/MS spectra were searched against the Uniprot human proteome database (January 2017) using Sequest HT in Proteome Discoverer V.1.4 pipeline. One fixed modification of propionamide at Cys (71.037114 Da) and three variable modifications, oxidation at Met (15.9949 Da) deamidation at Arg (0.984016 Da) and SILAC 13C6 at Lys (6.0201 Da), were chosen. The mass error allowed was 10 parts per million (ppm) for parent monoisotopic and 0.5 Da for tandem mass (MS2) fragment monoisotopic ions. Full trypsin was specified as protein cleavage site, with possibility of two missed cleavages allowed. The searched result was filtered with false discovery rate (FDR)=0.01, and the peptides with deamidated Arg at C-terminal of the tryptic peptides were considered as false identification and removed as well.

### B. Plasma Ig-Bound Citrullinome Analysis

Clinical subjects for Ig-bound analysis. Plasma samples were collected from 156 women with newly diagnosed breast cancer (0-0.8 years) as cases, and 40 age-matched cancer-free women were used as control. For the cases, only patients who had no documented distant metastasis at the time of sample collection were included in this study. Written informed consent was obtained and the study was approved by the IRB at MD Anderson Cancer Center. The timing of blood draw was after the diagnostic biopsy and prior to neoadjuvant chemotherapy, or definitive surgery in patients who did not receive chemotherapy in the neoadjuvant setting (ref. 23) (Table 6 and Table 8). The additional 73 healthy control plasmas were obtained from MD Anderson Cancer Center Gynecologic Tissue Bank following Institutional Review Board approval and informed consent (Table 6).

Plasma Ig-bound work flow. Plasma Ig-bound proteins were prepared and analyzed as described previously (ref. 24). Twenty six pooled plasma samples from 156 women with newly diagnosed breast cancer and 12 pooled plasma samples from 113 cancer-free subjects as controls were analyzed (Table 6). Briefly, 100 μL of pooled plasma for each experimental condition was processed with the immuno-depletion column Hu-14 10×100 mm (#5188-6559; Agilent Technologies, Santa Clara, California, USA) to remove the top 14 high abundance proteins, Albumin, IgG, IgA, Transferrin, Haptoglobin, Fibrinogen, al-Antitrypsin, al-Acid Glycoprotein, Apolipoprotein AI, Apolipoprotein AII, Complement C3, Transthyretin, IgM, and α2-Macroglobulin. The bound fraction was used for IgG-bound protein analysis as previously described (refs. 24 and 25).

LC-high-definition MSE (HDMSE) Data were acquired in resolution mode with SYNAPT G2-S using Waters Masslynx (V.4.1, SCN851). The capillary voltage was set to 2.80 kV, sampling cone voltage to 30 V, source offset to 30 V, and source temperature to 100° C. Mobility used high-purity N2 as the drift gas in the ion-mobility spectrometry (IMS) TriWave cell. Pressures in the helium cell, Trap cell, IMS TriWave cell, and Transfer cell were 4.50 mbar, 2.47e-2 mbar, 2.90 mbar, and 2.53e-3 mbar, respectively. The IMS wave velocity was 600 m/s, the helium cell DC was 50 V, the trap DC bias was 45 V, the IMS TriWave DC bias was 3 V, and the IMS wave delay was 1000 μs. The mass spectrometer was operated in V-mode with a typical resolving power of at least 20,000. All analyses were performed using positive mode electrospray ionization (ESI) using a NanoLockSpray source. The lock mass channel was sampled every 60 s. The mass spectrometer was calibrated with a [Glu1]-fibrinopeptide solution (300 fmol/μL) delivered through the reference sprayer of the NanoLockSpray source. Accurate mass LC-HDMSE data were collected in an alternating, low energy (MS) and high energy (MSE) mode of acquisition with mass scan range from m/z 50 to 1800. The spectral acquisition time in each mode was 1.0 s with a 0.1-s inter-scan delay. In low energy HDMS mode, data were collected at constant collision energy of 2 eV in both Trap cell and Transfer cell. In high energy HDMSE mode, the collision energy was ramped from 25 to 55 eV in the Transfer cell only. The RF applied to the quadrupole mass analyzer was adjusted such that ions from m/z 300 to 2000 were efficiently transmitted, ensuring that any ions observed in the LC-HDMSE data less than m/z 300 were known to arise from dissociations in the Transfer collision cell.

The acquired LC-HDMSE data were processed and searched against the Uniprot human proteome database (January 2017) through ProteinLynx Global Server (PLGS, Waters Company) with False Discovery Rate 4%. The modification search settings and the deamidated Arg, C-terminal miss cleavage assessment were the same with the cell line citrullinome. The plot values are represented as the number of citrullinated proteins relative to the total unique peptides per sample as to adjust for batch effects that occurred during the data acquisition. The Ingenuity Pathway Analysis (IPA) network analysis of plasma IgG-bound was done by calculating the ratio of the spectral counts of the citrullinome in each BC receptor subtype compared with healthy controls with greater than or equal to 1.5.

### C. Plasma Autoantibody Western Blot Assay

The recombinant unmodified Vimentin (Cayman Chemical, #11234) was incubated with 100 mM HEPES (pH7.6) containing 100 mM NaCl and 1 mM CaCl₂) buffer and stored under room temperature for 1 hour, and the citrullinated form was prepared. Each of the recombinant unmodified Vimentin and citrullinated Vimentin was loaded 0.1, 0.5, 1.0 μg to Criterion XT 12% gel and transferred to PVDF membrane using Trans-Blot Turbo Transfer System (BioRad, #1704150). From the Healthy control (n=8 pooled) and triple negative breast cancer (TNBC) stage II (n=11 pooled), 2 μL of the plasmas was diluted 150-fold with 0.05% casein dissolved in Tris buffered saline containing 0.01% Tween 20 (TBST) and incubated with the recombinant proteins transferred PVDF membrane for 2 hours, respectively. Then after washing the membrane with TBST, the secondary antibody of ECL anti-human IgG horseradish peroxidase-linked whole Ab (GE Healthcare #NA933) was added and stored for 1 hour under room temperature followed by Clarity Western ECL (BioRad, #170-5061) detection. The band intensities were read by ImageJ V.1.46r (imagej.nih.gov/).

### D. Plasma Autoantibody ELISA Assay

Clinical subjects for the plasma ELISA assay. Eleven newly diagnosed stage II TNBC patient plasmas and 31 healthy controls were used for autoantibody assays. Case plasmas were derived from a subset of patients in the breast cohort that were used for proteomic analyses of Ig-bound proteins that had sufficient volume. The independent set of healthy control plasmas was obtained from the MD Anderson Cancer Center Gynecologic Tissue Bank as described above (Table 6 and Table 8).

Plasma autoantibody assay method. Concentrations for anti-Vimentin, anti-citrullinated Vimentin autoantibodies were determined using Luminex bead-based immunoassays on the MAGPIX instrument (Luminex Corporation, Austin Texas). Samples were analyzed in the same batch in random order. MagPlex Microspheres were conjugated with purified recombinant Vimentin (Cayman Chemical, #11234) and recombinant Citrullinated Vimentin (Cayman Chemical, #21942) at 5 μg/million concentration. The samples were tested at the final dilution of 1:16,250. The acid dissociated autoantibodies were prepared by 5 μL plasma diluted 50-fold with 0.1 M Gly-HCl (pH 3.0), stored under room temperature for 30 minutes, and exchanged buffer to Reagent Diluent Concentrate 2 buffer (R&D Systems, #841380) using Zeba spin column (ThermoFisher Scientific, #89890). The samples were incubated with MagPlex beads for 2 hrs at room temperature on a shaker. After the samples were washed with PBST, PE conjugated secondary antibody was used to incubate samples for 30 mins. The measured fluorescent intensity (MFI) of each well was read using MAGPIX instrument. The TNBC stage II plasmas in Table 6 were used for the assay and the anonymous individual subject information is presented in Table 8.

### E. Cell Surface Human Leukocyte Antigen (HLA)-Bound Peptidyl-Citrullinome

A total of 5×10′ HCC1954 and TNBC MDA-MB-468 cells were used for culture in peptidyl-citrullinome analysis using the LTQ Orbitrap ELITE as described previously (ref. 26). The MS/MS spectra were searched against the Uniprot human proteome database (January 2017) using Sequest HT in Proteome Discoverer V.1.4 pipeline. The search was conducted for amino acid lengths from 8 to 34. Two variable modifications, oxidation at Met (15.9949 Da) and deamidation at Arg (0.984016 Da), were chosen. The mass error allowed was 10 ppm for parent monoisotopic and 0.5 Da for MS2 fragment monoisotopic ions. The searched result was filtered with FDR=0.01. The identified peptides with amino acid lengths putatively from 8 to 11 were considered as MHC-I binding peptides and from 12 to 34 as MHC-IU binding peptides.

We additionally performed in silico binding affinity predictions between peptides and MHC-II molecules using the well stablished prediction tool NetMHC-II pan V.2.3 (refs. 27 and 28). Briefly, identified peptide sequences were loaded to the prediction tool NEetMHC-II pan2.3, then the data were sorted by the binding peptide core affinity prediction (IC50, nM) with MHC-II pocket assessed by the artificial intelligent network SSNAligment, and the percentile rank that is generated by comparing the peptide's score against the scores of one million random 15 mers selected from Swiss-Prot database.

F. PADI2 siRNA Knockdown

Transient knockdown of PADI2 was performed by transfecting cells with 50 nM of siControl (Silencer Select Negative Control No. 1, Thermo Fisher Scientific), siPADI2#1 (s22187, Thermo Fisher Scientific) and siPADI2#2 (s22188, Thermo Fisher Scientific) in Lipofectamine RNAiMAX (Thermo Fisher Scientific). After 72 hours of transfection, total RNA was isolated using RNeasy Mini Kit (Qiagen, Germantown, Maryland). Reverse transcription was performed with 1 ug of total RNA using High-Capacity cDNA Reverse Transcription Kit (Thermo Fisher Scientific) and real time PCR performed using TaqMan Gene Expression Assays (Hs00247108_ml, PADI2 FAM-MGB, Thermo Fisher Scientific). 18S (Hs99999901_s1, VIC-MGB, Thermo Fisher Scientific) was used as an internal control. All samples were assayed in triplicate.

Proteins were extracted using (8M Urea and 50 mM Triethylammonium bicarbonate (TEAB) with protease inhibitor cocktail (complete Protease Inhibitor Cocktail, Millipore Sigma). A total of 100 ug protein was used for tandem mass tag (TMT) labeling per channel. After reduction with TCEP (Tris(2-carboxyethyl)phosphine) and alkylation with acrylamide, proteins were digested with Lys-C (WAKO) overnight at 37° C. Digested peptides were desalted with Monospin C18 column (GL Sciences, Tokyo, Japan) and dried by speedvac. The tryptic peptides were re-suspended in 0.1M TEAB buffer/acetonitrile and reacted with each TMT channel for one hour (TMTsixplex Isobaric Label Reagent Set, Thermo Fisher) and then quenched with hydroxylamine, mixed together and dried by speedvac. Tryptic peptides were subsequently fractionated under high-pH conditions using Monospin L C18 column (GL Sciences) in 0.1% trimethylamine/acetonitrile with step elution of acetonitrile into 10 fractions.

Fractionated peptides were injected into an EASYnano HPLC system (Thermo Fisher Scientific) online coupled with Q Exactive Mass Spectrometer (Thermo Fisher Scientific). The system was equipped with a Waters Symmetry C18 nanoAcquity trap-column (180 μm×20 mm, 5 μm) and a C18 analytical column (75 μm×200 mm, 3 μm; Column Technology). The separation column temperature was set ambient, and the temperature of the tray compartment in the auto-sampler was set at 6° C. Mass spectrometer parameters were spray voltage 2.5 kV, capillary temperature 320° C., Fourier transform (FT) resolution 70,000, AGC target value 3×10⁶, 1 microscan with 30 ms injection time. Mass spectra were acquired in a data-dependent mode in the m/z range of 350-1,800. The step gradient of Normalized Collision Energy (NCE) 20, 25, 35 was applied to induce fragmentation. Acquisition of each full mass spectrum was followed by acquisition of MS/MS spectra for the 10 most intense +2, +3 or +4 ions within a duty cycle. The acquired LC-MS/MS data were processed by the Proteome Discoverer V.1.4 (Thermo Scientific). Sequest HT was used as a search engine with the parameters including fixed modification of Cys alkylated with acrylamide (+71.03714), Lys with TMT (+229.162932, N-terminal and Lys), and variable modification of Met oxidation (+15.99491) and Arg deamidation (+0.984016). Mass error of 10 ppm was allowed for the parent MS1 and 0.02 Da was allowed for the MS2 fragments. Data were searched against the Uniprot human database (2017) and were further filtered with FDR=0.01 and TMT ratios were quantified.

### G. Immunohistochemistry (IHC)

Clinical subjects for breast cancer tissue microarray analysis. Breast cancer tissue microarrays (TMA; BC081120C, BR20810, and BR20811) including 127 Luminal A, 99 Luminal B, 57 HER2-enriched, and 139 TNBC from a total of 422 patient tissues, were used for assessment of PADI2 expression related to clinical parameters and co-expression with peptidyl-citrulline (Table 3).

IHC work flow. Healthy tissue microarray (TMA; BN0001a) and breast cancer tissue sections (HuCAT297, HuCAT298, 2017-16604A TNBC, Fmg0105378 Her2+, and Fmg030209B5 ER+) were purchased from US Biomax (Rockville, Maryland, USA) and healthy mammary gland tissue section was obtained from Zyagen (HP-414). Sections were de-paraffinized in xylene, rehydrated in a descending ethanol series, and then treated with 3% hydrogen peroxide for 10 min. Antigen retrieval was conducted in a pressure cooker in 1× ImmunoDNA Retriever with citrate (Bio SB, Santa Barbara, California, USA) and 0.1% Tween 20 at 121° C. for 15 min. Sections were hybridized with 1:2000-times diluted anti-PADI2 monoclonal antibody (66386-1-1g, Proteintech, Rosemont, Illinois, USA), 1:1000-times diluted anti-citrulline monoclonal antibody (Clone F95, Millipore Sigma, Burlington, Massachusetts, USA), 1:250 times diluted anti-CD20cy monoclonal antibody (Clone L26, Agilent Technologies), 1:50 times diluted anti-CD19 monoclonal antibody (Clone LE-CDI9, Agilent Technologies), and 1:1000 times diluted anti-pan Cytokeratin monoclonal antibody (Clone AE1/AE3+5D3, Abcam, Cambridge, UK) for 16 hrs at 4° C. After washing with TBS for 5 min×3, signal development was performed by Histofine DAB-2V kit (Nichirei Bioscience, Tokyo, Japan). Images were scanned by MD Anderson Cancer Center (MDACC)-North Campus Research Histology Core Laboratory and analyzed using Aperio Imagescope (Leica Biosystems, Buffalo Grove, Illinois, USA). For PADI2 and Citrulline IHC evaluation was independently performed by three operators. Positivity of cancer cells was scored as Low: 0-24%, Low-Moderate: 25-49%, Moderate-High: 50-74%, and High: 75-100/c and p-values were calculated by 2-sided X²-square test for the trend between PADI2 staining positivity and the respective comparative groups. Since BR20810 and BR20811 consisted of duplicate tissues per patient, we derived an average score.

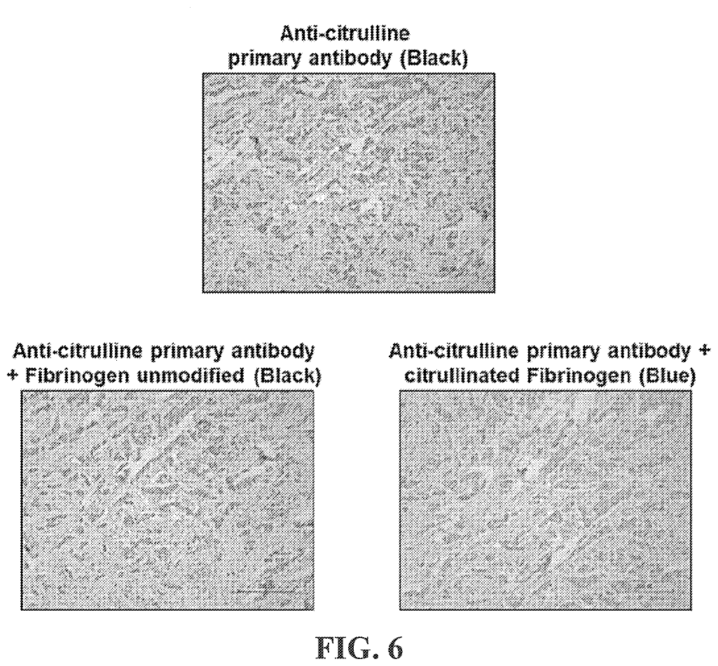

For antigen absorption based IHC, we used a previously developed method (ref. 29) with slight modification. Anti-PADI2 antibody was 2,000× and mixed with various concentrations of recombinant citrullinated fibrinogen (18473, Cayman Chemical) and uncitrullinated fibrinogen (16088, Cayman Chemical) and incubated at 4° C. overnight. After centrifugation at 20,000×g for 30 min, the supernatant was used as primary antibody for IHC. Reactivity of the antibody was confirmed using breast invasive ductal carcinoma tissue sections and the positive staining was observed in the presence of unmodified fibrinogen whereas staining was abrogated in the presence of citrullinated fibrinogen (FIG. 6).

### H. Gene Expression Data

The Cancer Genome Atlas (TCGA) gene expression data, HM450 methylation data, and clinical data were downloaded from cBioPortal (ref. 30). Gene expression for the Curtis dataset (ref. 31) was obtained through the Oncomine database (ref. 32).

### I. Immune Cell Signature Analyses

Immune signatures were derived as previously described (ref. 33). Briefly, specific immune cell infiltration was computationally inferred using RNA-seq data based on gene sets overexpressed in one of 24 immune cell types according to Bindea et al. (ref. 34). Scoring of TCGA cancer samples for each of the immune cell signatures and for expression of Antigen Presentation MHC class I (APM1) genes (HLA-A/B/C, p2M, TAP1/2, TAPBP) or Antigen Presentation MHC class II (APM2) genes (HLA-DR/DQ/DP/DM) is described elsewhere (ref. 35).

### J. Statistical Analyses

Unsupervised hierarchical clustering heatmaps were generated using R statistical software. Figures were generated using R statistical software or GraphPad Prism V.8. Spearman's correlation analyses were performed to assess relationships between continuous variables. Fisher's exact tests were used to assess relationships between categorical variables. All statistical tests were two-sided unless specified otherwise.

### Example 2. PADI2 is Highly Expressed in Breast Cancer Among Various Cancer Types

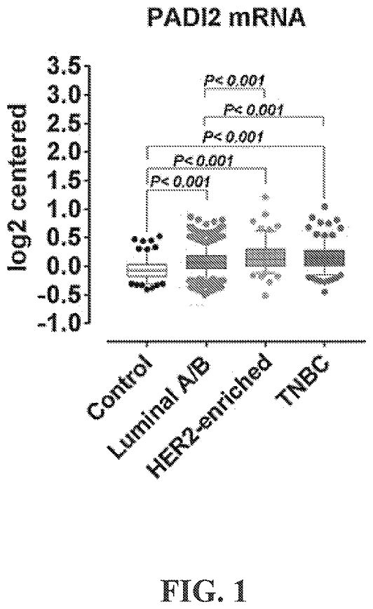

Proteomic analysis of PADI family protein expression in whole cell lysates from 196 cancer cell lines were stratified by cancer type of brain, breast, colon, gastric, glioma, leukemia, small cell lung, non-small cell lung, melanoma, ovarian, pancreatic and prostate cancer. The quantitative expression was based on the spectral counts of the peptides in common as well as unique sequences that distinct PADI family members. The PADI2 protein expression was highest among other family members and relatively enriched in breast cancer (data not shown). Using mRNA expression datasets from TCGA, we further interrogated differential expression of PADI family members amongst 9,721 human tumors consisting of 32 different cancer types including ACC: Adrenocortical carcinoma, BLCA: Bladder Urothelial Carcinoma, BRCA: Breast invasive carcinoma, CESC: Cervical squamous cell carcinoma and endocervical adenocarcinoma, CHOL: Cholangiocarcinoma, COAD: Colon adenocarcinoma, DLBC: Lymphoid Neoplasm Diffuse Large B-cell Lymphoma, ESCA: Esophageal carcinoma, GBM: Glioblastoma multiforme, HNSC: Head and Neck squamous cell carcinoma, KICH: Kidney Chromophobe, KIRC: Kidney renal clear cell carcinoma, KIRP: Kidney renal papillary cell carcinoma, LAML: Acute Myeloid Leukemia, LGG: Brain Lower Grade Glioma, LIHC: Liver Hepatocellular carcinoma, LUAD: Lung adenocarcinoma, LUSC: Lung squamous cell carcinoma, MESO: Mesothelioma, OV: Ovarian serous cystadenocarcinoma, PAAD: Pancreatic adenocarcinoma, PCPG: Pheochromocytoma and Paraganglioma, PRAD: Prostate adenocarcinoma, SARC: Sarcoma, SKCM: Skin Cutaneous Melanoma, STAD: Stomach adenocarcinoma, TGCT: Testicular Germ Cell Tumors, THCA: Thyroid carcinoma, THYM: Thymoma, UCEC: Uterine Corpus Endometrial Carcinoma, UCS: Uterine carcinosarcoma, UVM: Uveal Melanom. Concordant with proteomic data, relative to the other PADI family members, PADI2 exhibited the highest mRNA expression levels (RNA Seq V2 RSEM) among the cancer types (data not shown). We determined the differential expression of PADI2 among breast cancer subtypes in 1,725 breast tumors and 144 normal breast tissues from the Curtis cohort (ref. 36). Breast tumors exhibited statistically significantly elevated mRNA expression of PADI2 compared with normal breast tissues with an increasing trend from Luminal A/B, HR-/HER2-enriched to TNBC tumors (Dunn's multiple comparison test, two-sided p<0.001, FIG. 1). Spearman's correlation analyses between PADI2 mRNA expression and HM450 methylation β-values from TCGA further indicated statistically significant inverse associations for most of the cancer types among the 32 cancer types described above, suggesting that PADI2 gene expression is in part, regulated through DNA methylation (data not shown).

### Example 3. Proteomics Revealed PADI2 Mediated Citrullinome in Breast Cancer

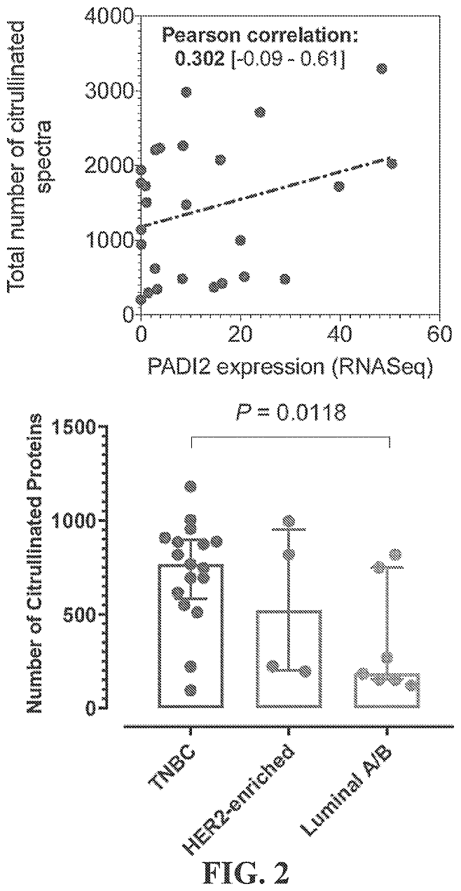

We next examined whether the extent of protein citrullination was reflective of differential expression of PADI2 among the breast cancer subtypes. Global analysis of the protein citrullinome in whole cell lysates of 28 breast cancer cell lines (8 Luminal A/B, 5 hormone receptor (HR)-/HER2-enriched, and 15 TNBC) by mass spectrometry with a strict criteria of FDR=0.01 revealed that the total number or citrullinated spectra in the cell lines had positive correlation with PADI2 expression level (FIG. 2, top panel, Pearson correlation=0.302) and TNBC had significantly higher number of citrullinated proteins compared with Luminal A/B (FIG. 2, bottom panel, P=0.0118). The representative citrullinome discriminated between the Luminal A/B, HR-/HER2-enriched and TNBC (ANOVA 2-sided p<0.05) were listed in Table 1. Of the citrullinated proteins identified in the whole cell lysates, their cellular localization was classified as Nucleus (22.8%), Cytoplasm (59.6%), Plasma Membrane (10.5%) and Extracellular Space (7.0%) based on Ingenuity Pathway Analysis (ingenuity.com). (ref. 37) (Table 1). We also identified citrullinated vimentin (Table 1) and citrullinated a-enolase (Table 2) which are known to occur in autoimmune diseases (refs. 38-40).

TABLE 1
IPA localization of breast cancer whole cell lysate citrullinome.

| Gene | IPA_Location | IPA_Types |
|---|---|---|
| ADCK1 | Cytoplasm | kinase |
| ALDH7A1 | Cytoplasm | enzyme |
| ARF5 | Cytoplasm | enzyme |
| ATP2A2 | Cytoplasm | transporter |
| ATP6V1A | Plasma | transporter |
| | Membrane | |
| CALD1 | Cytoplasm | other |
| CAPNS1 | Cytoplasm | peptidase |
| CCT2 | Cytoplasm | kinase |
| CEP128 | Cytoplasm | other |
| CLNS1A | Plasma | ion channel |
| | Membrane | |
| COL1A1 | Extracellular | other |
| | Space | |
| CRK | Cytoplasm | other |
| CTTN | Plasma | other |
| | Membrane | |
| DBI | Cytoplasm | other |
| DNAJC8 | Nucleus | other |
| DPP3 | Cytoplasm | peptidase |
| EIF4G1 | Cytoplasm | translation |
| | | regulator |
| EPRS | Cytoplasm | enzyme |
| EZR | Plasma | other |
| | Membrane | |
| FKBP4 | Nucleus | enzyme |
| G6PD | Cytoplasm | enzyme |
| GPI | Extracellular | enzyme |
| | Space | |
| GPR161 | Plasma | G-protein |
| | Membrane | coupled |
| | | receptor |
| HADHB | Cytoplasm | enzyme |
| HDLBP | Nucleus | transporter |
| HSP90AB1 | Cytoplasm | enzyme |
| HSPD1 | Cytoplasm | enzyme |
| IQGAP1 | Cytoplasm | other |
| KRT15 | Cytoplasm | other |
| KRT19 | Cytoplasm | other |
| KRT7 | Cytoplasm | other |
| LONP1 | Cytoplasm | peptidase |
| LRPPRC | Cytoplasm | other |
| MSN | Plasma | other |
| | Membrane | |
| MVP | Nucleus | other |
| MYH14 | Extracellular | enzyme |
| | Space | |
| PAWR | Nucleus | transcription |
| | | regulator |
| PFN1 | Cytoplasm | other |
| PGAM1 | Cytoplasm | phosphatase |
| PGRMC2 | Nucleus | other |
| PSMA1 | Cytoplasm | peptidase |
| RAB1B | Cytoplasm | other |
| RCN1 | Cytoplasm | other |
| RHOA | Cytoplasm | enzyme |
| RPN1 | Cytoplasm | enzyme |
| SERPINH1 | Extracellular | other |
| | Space | |
| SFN | Cytoplasm | other |
| SFPQ | Nucleus | transcription |
| | | regulator |
| SMARCC2 | Nucleus | transcription |
| | | regulator |
| SNRPD1 | Nucleus | other |
| SRSF1 | Nucleus | other |
| TAF15 | Nucleus | other |
| UBQLN2 | Nucleus | other |
| VCP | Cytoplasm | enzyme |
| VIM | Cytoplasm | other |
| XRCC6 | Nucleus | enzyme |
| YWHAE | Cytoplasm | other |

TABLE 2
Breast cancer cell surface MHC binding peptidyl-citrullinome.

| | | | MIDA- | | Net MHCIIpan-2.3 prediction of anmodified form peptides | | | |
|---|---|---|---|---|---|---|---|---|
| | | | MB-468 | HCC1954 | | Affinity | | |
| Gene | Sequence | Modification | (TNBC) | (HR−/Her2+) | MHC II allele | (nM) | %Rank | Bind Level |
| AIMP2 | YQVKPYHGGGAPLR*VEL | R14(Cit.) | MHC-II | | DRB5_0101 | 285.5 | 8.5 | Moderate |
| | PT (SEQ ID NO: 1) | | | | | | | |
| API5 | SGLKSLQTVSGR*QQL | R12(Cit.) | MHC-II | | DRB1_0101 | 15.5 | 1.6 | Strong |
| | (SEQ ID NO: 2) | | | | | | | |
| ARPC2 | NRAR*PDAEKKEMKTITG | R4(Cit.) | MHC-II | | DRB1_0101 | 20 | 2.5 | Moderate |
| | KTFSSR (SEQ ID NO: 3) | | | | | | | |
| ARPC3 | VKR*QFMNKSLSGPGQ | R3(Cit.) | MHC-II | | | | | |
| | (SEQ ID NO: 4) | | | | | | | |
| ATL3 | YVLEQASSHIGNSTQATVR | R19(Cit.); | MHC-II | | DRB5_0101 | 282.4 | 8.5 | Moderate |
| | *DAVVGR*PSMDKKAQ | R25(Cit.) | | | | | | |
| | (SEQ ID NO: 5) | | | | | | | |
| ATL3 | YVLEQASSHIGNSTQATVR | R19(Cit.) | MHC-II | | | | | |
| | *DAVVGRPSMDKKAQ | | | | | | | |
| | (SEQ ID NO: 6) | | | | | | | |
| ATP5O | LAENGR*LSNTQGVVSAFS | R6(Cit.) | MHC-II | | | | | |
| | (SEQ ID NO: 7) | | | | | | | |
| ATP5O | LAENGR*LSNTQGVVSAFS | R6(Cit.) | MHC-II | | | | | |
| | TM (SEQ ID NO: 8) | | | | | | | |
| ATP6V | IAQQMMPEVR*GALFGAN | R10(Cit.) | MHC-II | | HLA- | 852.5 | 1.9 | Strong |
| 1E1 | ANRKF (SEQ ID NO: 9) | | | | DPA10103- | | | |
| | | | | | DPB10301 | | | |
| | | | | | DRB5_0101 | 62.6 | 3 | Moderate |
| | | | | | HLA- | 3365.3 | 7 | Moderate |
| | | | | | DPA10103- | | | |
| | | | | | DPB10301 | | | |
| B2M | LLKNGER*IEKVEHSDLSF | R7(Cit.) | MHC-II | | | | | |
| | SKDWS (SEQ ID NO: 10) | | | | | | | |
| CALM1 | MAR*KMKDTDSEEEIREA | R3(Cit.) | | MHC-II | | | | |
| | (SEQ ID NO: 11) | | | | | | | |
| CALM1 | MIR*EADIDGDGQVNYEE | R3(Cit.) | MHC-II | | | | | |
| | (SEQ ID NO: 12) | | | | | | | |
| CALM1 | IR*EADIDGDGQVNYEE | R2(Cit.) | MHC-II | | | | | |
| | (SEQ ID NO: 13) | | | | | | | |
| CALM1 | IR*EADIDGDGQVNYE | R2(Cit.) | MHC-II | | | | | |
| | (SEQ ID NO: 14) | | | | | | | |
| CALM1 | DKDGNGYISAAELR*HVM | R14(Cit.) | MHC-II | | DRB5_0101 | 86.9 | 3.5 | Moderate |
| | (SEQ ID NO: 15) | | | | | | | |
| CALML5 | MIREADVDQDGR*VNYEE | R12(Cit.) | MHC-II | | | | | |
| | (SEQ ID NO: 16) | | | | | | | |
| CALML5 | MIREADVDQDGR*VNYEE | R.12(Cit.) | MHC-II | | | | | |
| | F (SEQ ID NO: 17) | | | | | | | |
| CALML5 | MIREADVDQDGR*VNYE | R12(Cit.) | MHC-II | | | | | |
| | (SEQ ID NO: 18) | | | | | | | |
| CALML5 | IREADVDQDGR*VNYE | R1I(Cit.) | MHC-II | | | | | |
| | (SEQ ID NO: 19) | | | | | | | |
| CAP1 | EAVSHTSDMHR*GYADSP | R11(Cit.) | MHC-II | | | | | |
| | SKAGAAPYVQAF (SEQ ID | | | | | | | |
| | NO: 20) | | | | | | | |
| CAPZA1 | NNDNLLR*EGAAHAFAQY | R7(Cit.) | MHC-II | | DRB1_0101 | 42.7 | 6 | Moderate |
| | (SEQ ID NO: 21) | | | | | | | |
| | | | | | HLA- | 4810.8 | 9.5 | Moderate |
| | | | | | DPA10103- | | | |
| | | | | | DPB10301 | | | |
| CBR1 | LPPDAEGPHGQFVSEKR*V | R17(Cit.) | MHC-II | | | | | |
| | E (SEQ ID NO: 22) | | | | | | | |
| CCT8 | IPR*ALAENSGVKANEVIS | R3(Cit.) | MHC-II | | | | | |
| | KL (SEQ ID NO: 23) | | | | | | | |
| CCT8 | R*VDQIIMAKPAGGPKPPS | RI(Cit.) | MHC-II | | DRB1_0101 | 12.9 | 1.2 | Strong |
| | GKKDWDDDQND (SEQ ID | | | | | | | |
| | NO: 24) | | | | | | | |
| | | | | | DRB5_0101 | 267.6 | 8 | Moderate |
| | | | | | HLA- | 1974.1 | 4 | Moderate |
| | | | | | DPA10103- | | | |
| | | | | | DPB10301 | | | |
| CHST2 | NPDGPLGAAAGAAGGSW | R19(Cit.) | | MHC-II | | | | |
| | GR*PGPPPA (SEQ ID | | | | | | | |
| | NO: 25) | | | | | | | |
| COBLL1 | VTRR*LGAGARAAPRR | R4(Cit.) | | MHC-II | HLA- | 219 | 0.5 | Strong |
| | (SEQ ID NO: 26) | | | | DPA10103- | | | |
| | | | | | DPB10301 | | | |
| | | | | | HLA- | 3601.9 | 7.5 | Moderate |
| | | | | | DPA10103- | | | |
| | | | | | DPB10301 | | | |
| COPS5 | KLEQSEAQLGR*GSF (SEQ | R11(Cit.) | MHC-II | | | | | |
| | ID NO: 27) | | | | | | | |
| COX5A | FNKPDIDAWELR*KGINTL | R12(Cit.) | MHC-II | | | | | |
| | (SEQ ID NO: 28) | | | | | | | |
| CSTB | FVHLR*VFQSLPHENKPLT | R5(Cit.) | MHC-II | | HLA- | 722 | 1.6 | Strong |
| | LSNYQTNKAKHDE (SEQ | | | | DPA10103- | | | |
| | ID NO: 29) | | | | DPB10301 | | | |
| | | | | | DRB5_0101 | 345.8 | 9.5 | Moderate |
| | | | | | HLA- | 4041.2 | 8 | Moderate |
| | | | | | DPA10103- | | | |
| | | | | | DPB10301 | | | |
| CSTB | R*VFQSLPHENKPLTLSNY | RI(Cit.) | MHC-II | | | | | |
| | QTNKAKHDE (SEQ ID | | | | | | | |
| | NO: 30) | | | | | | | |
| DUT | EVQALDDTER*GSGGFGST | R.10(Cit.) | MHC-II | | | | | |
| | GKN (SEQ ID NO: 31) | | | | | | | |
| DYNC112 | INANR*ADAEEEAATRIPA | R5(Cit.) | MHC-II | | | | | |
| | (SEQ ID NO: 32) | | | | | | | |
| DYNLRB1 | LR*IRSKKNEIMVAPDKDY | R2(Cit.) | MHC-II | | | | | |
| | FL (SEQ ID NO: 33) | | | | | | | |
| EEF1A1 | WKVTR*KDGNASGTTLL | R5(Cit.) | MHC-II | MHC-II | | | | |
| | (SEQ ID NO: 34) | | | | | | | |
| EEF1A1 | WKVTR*KDGNASGTTL | R5(Cit.) | MHC-II | | | | | |
| | (SEQ ID NO: 35) | | | | | | | |
| EEF1A1 | WKVTR*KDGNASGTTLLE | R5(Cit.) | MHC-II | | | | | |
| | (SEQ ID NO: 36) | | | | | | | |
| EEF1A1 | YVTIIDAPGHR*DFIKNM | R11(Cit.) | | MHC-II | DRB5_0101 | 159.9 | 5.5 | Moderate |
| | (SEQ ID NO: 37) | | | | | | | |
| EEF1A1 | R*QTVAVGVIKAVDKKAA | RI(Cit.) | MHC-II | | DRB5_0101 | 39.5 | 1.8 | Strong |
| | GAGKVTKSAQKAQKAK | | | | | | | |
| | (SEQ ID NO: 38) | | | | | | | |
| EEF1G | YVSNEELR*GSTPEAAAQV | R8(Cit.) | MHC-II | | HLA- | 665 | 1.5 | Strong |
| | VQ (SEQ ID NO: 39) | | | | DPA10103- | | | |
| | | | | | DPB10301 | | | |
| | | | | | HLA- | 1127 | 2.5 | Moderate |
| | | | | | DPA10103- | | | |
| | | | | | DPB10301 | | | |
| EEF1G | YVSNEELR*GSTPEAAAQV | R8(Cit.) | MHC-II | | | | | |
| | VQWVS (SEQ ID NO: 40) | | | | | | | |
| ENO1 | FKSPDDPSR*YISPDQLADL | R9(Cit.) | MHC-II | MHC-II | | | | |
| | (SEQ ID NO: 41) | | | | | | | |
| ENO1 | FTSKGLFR*AAVPSGASTG | R8(Cit.) | MHC-II | | DRB1_0101 | 14.9 | 1.5 | Strong |
| | IYE (SEQ ID NO: 42) | | | | | | | |
| ENO1 | GVMVSHR*SGETEDTF | R7(Cit.) | | MHC-II | | | | |
| | (SEQ ID NO: 43) | | | | | | | |
| ENO1 | LAQANGWGVMVSHR*SG | R14(Cit.) | | MHC-II | DRB5_0101 | 335.2 | 9.5 | Moderate |
| | ETEDTF (SEQ ID NO: 44) | | | | | | | |
| ENO1 | AVEKGVPLYR*HIADLAG | R10(Cit.) | | MHC-II | HLA- | 694.5 | 1.5 | Strong |
| | NS (SEQ ID NO: 45) | | | | DPA10103- | | | |
| | | | | | DPB10301 | | | |
| | | | | | HLA- | 3609.6 | 7.5 | Moderate |
| | | | | | DPA10103- | | | |
| | | | | | DPB10301 | | | |
| FUBP1 | YR*QQAAYYAQTSPQGMP | R2(Cit.) | MHC-II | | DRB1_0101 | 53 | 7.5 | Moderate |
| | QHPPAPQGQ (SEQ ID | | | | | | | |
| | NO: 46) | | | | | | | |
| | | | | | HLA- | 3361.9 | 7 | Moderate |
| | | | | | DPA10103- | | | |
| | | | | | DPB10301 | | | |
| GAPDH | TVHAITATQKTVDGPSGK | R21(Cit.) | | MHC-II | DRB5_0101 | 24.8 | 1.1 | Strong |
| | LWR* (SEQ ID NO: 47) | | | | | | | |
| | | | | | DRB5_0101 | 100 | 4 | Moderate |
| GAPDH | ISWYDNEFGYSNR*VVDL | R13(Cit.) | MHC-II | | | | | |
| | (SEQ ID NO: 48) | | | | | | | |
| GAPDH | ISWYDNEFGYSNR*VVD | R13(Cit.) | MHC-II | | | | | |
| | SEQ ID NO: 49) | | | | | | | |
| GAPDH | WYDNEFGYSNR*VVD | RII(Cit.) | MHC-II | | | | | |
| | (SEQ ID NO: 50) | | | | | | | |
| GAR1 | GGGNFRGGGRGGFGRGG | R.19(Cit.) | | MHC-II | | | | |
| | GR*GGFNKGQ (SEQ ID | | | | | | | |
| | NO: 51) | | | | | | | |
| GBA | SLTGLLLLQAVSWASGAR | R18(Cit.) | MHC-II | | DRB5_0101 | 345.3 | 9.5 | Moderate |
| | *P (SEQ ID NO: 52) | | | | | | | |
| HDDC2 | ELEAER*STNIAAAASEPH | R6(Cit.) | MHC-II | | HLA- | 611.3 | 1.4 | Strong |
| | S (SEQ ID NO: 53) | | | | DPA10103- | | | |
| | | | | | DPB10301 | | | |
| | | | | | HLA- | 4782.3 | 9.5 | Moderate |
| | | | | | DPA10103- | | | |
| | | | | | DPB10301 | | | |
| HIBADH | MDAPVSGGVGAAR*SGNL | R.13(Cit.) | MHC-II | | | | | |
| | TF (SEQ ID NO: 54) | | | | | | | |
| HISTI | VR*LLLPGELAKHAVSEGT | R2(Cit.) | MHC-II | | DRB1_0101 | 10.4 | 0.8 | Strong |
| H2BO | KAVTKYTSSK (SEQ ID | | | | | | | |
| | NO: 55) | | | | | | | |
| | | | | | HLA- | 2641.4 | 5.5 | Moderate |
| | | | | | DPA10103- | | | |
| | | | | | DPB10301 | | | |
| HMGCSI | YSPQQLAGKR*IGVFSYGS | R10(Cit.) | MHC-II | | DRB1_0101 | 69.7 | 9.5 | Moderate |
| | GLAA (SEQ ID NO: 56) | | | | | | | |
| | | | | | HLA- | 4136.3 | 8.5 | Moderate |
| | | | | | DPA10103- | | | |
| | | | | | DPB10301 | | | |
| HMGN1 | PKRKVSSAEGAAKEEPKR | R19(Cit.) | MHC-II | | DRB5_0101 | 277.3 | 8.5 | Moderate |
| | R*SARLS (SEQ ID NO: 57) | | | | | | | |
| | | | | | HLA- | 2799.1 | 6 | Moderate |
| | | | | | DPA10103- | | | |
| | | | | | DPB10301 | | | |
| HNRNPA1 | FAKPR*NQGGYGGSSSSSS | R5(Cit.); | MHC-II | | | | | |
| | YGSGR*RF (SEQ ID NO: 58) | R23(Cit.) | | | | | | |
| HSPB1 | SR*AQLGGPEAAKSDETA | R2(Cit.) | MHC-II | | | | | |
| | AK (SEQ ID NO: 59) | | | | | | | |
| IL16 | GIYVKTIFAGGAAAADGR* | R18(Cit.) | MHC-II | | DRB1_0101 | 71.5 | 9.5 | Moderate |
| | LQEGDE (SEQ ID NO: 60) | | | | | | | |
| | | | | | HLA- | 4419 | 9 | Moderate |
| | | | | | DPA10103- | | | |
| | | | | | DPB10301 | | | |
| IPO4 | QIR*QFAAVLTR*RRLNTR | R3(Cit.); | | MHC-II | DRB5_0101 | 23.6 | | Strong |
| | WRRLAAEQRESLKSL | RII(Cit.) | | | | | | |
| | (SEQ ID NO: 61) | | | | | | | |
| | | | | | HLA- | 28.1 | 0.04 | Strong |
| | | | | | DPA10103- | | | |
| | | | | | DPB10301 | | | |
| | | | | | DRB5_0101 | 80.8 | 3.5 | Moderate |
| | | | | | HLA- | 4493.6 | 9 | Moderate |
| | | | | | DPA10103- | | | |
| | | | | | DPB10301 | | | |
| KCTD8 | QWIPPPDKR*RNSELFQ | R9(Cit.) | MHC-II | MHC-II | | | | |
| | (SEQ ID NO: 62) | | | | | | | |
| KRT8 | R*GGLGGGYGGASGMGGI | R1(Cit.); | | | | | | |
| | TA (SEQ ID NO: 63) | M14(Oxi.) | | | | | | |
| LDHA | LR*TPKIVSGKDYNVTANS | R2(Cit.) | MHC-II | | DRB5_0101 | 300.9 | 9 | Moderate |
| | KL (SEQ ID NO: 64) | | | | | | | |
| MAGEF1 | ALADEADR*ARAKA (SEQ | R8(Cit.) | | | | | | |
| | ID NO: 65) | | | | | | | |
| M1F | VNTNVPR*ASVPDGELSEL | R7(Cit.) | MHC-II | | | | | |
| | (SEQ ID NO: 66) | | | | | | | |
| MK167 | LALRKR*TPSAGKAMDTP | R6(Cit.) | MHC-II | | DRB5_0101 | 324.4 | 9 | Moderate |
| | KPAVSDEKNINTF (SEQ ID | | | | | | | |
| | NO: 67) | | | | | | | |
| | | | | | HLA- | 2418.7 | 5 | Moderate |
| | | | | | DPA10103- | | | |
| | | | | | DPB10301 | | | |
| NDUFA4 | WDR*NNPEPWNKLGPND | R3(Cit.) | MHC-II | | | | | |
| | QYK (SEQ ID NO: 68) | | | | | | | |
| NME2 | NVVKTGR*VMLGETNPAD | R7(Cit.); | MHC-II | | | | | |
| | SKPGTIR*GD (SEQ ID | R24(Cit.) | | | | | | |
| | NO: 69) | | | | | | | |
| NME2 | NVVKTGR*VMLGETNPAD | R7(Cit.) | MHC-II | | | | | |
| | SKPGTIRGDF (SEQ ID | | | | | | | |
| | NO: 70) | | | | | | | |
| NME2 | NVVKTGR*VMLGETNPAD | R7(Cit.) | MHC-II | | | | | |
| | SKPGTIRGD (SEQ ID | | | | | | | |
| | NO: 71) | | | | | | | |
| NME2 | NVVKTGR*VMLGETNPAD | R7(Cit.) | MHC-II | | | | | |
| | (SEQ ID NO: 72) | | | | | | | |
| NUMB | WAALENKSKQR*TNPSPT | R11(Cit.) | MHC-II | | DRB5_0101 | 95.6 | 4 | Moderate |
| | NPFSSDLQK (SEQ ID | | | | | | | |
| | NO: 73) | | | | | | | |
| NUMB | WAALENKSKQR*TNPSPT | RII(Cit.) | MHC-II | | | | | |
| | NPFSSDLQKT (SEQ ID | | | | | | | |
| | NO: 74) | | | | | | | |
| PA2G4 | LMPNGPMR*ITSGPFEPDL | R8(Cit.) | MHC-II | MHC-II | HLA- | 4557.1 | 0 | Moderate |
| | (SEQ ID NO: 75) | | | | DPA10103- | | | |
| | | | | | DPB10301 | | | |
| PCBP1 | YSIQGQHTISPLDLAKLNQ | R22(Cit.) | MHC-II | | DRB1_0101 | 60.1 | 8 | Moderate |
| | VAR*QQSH (SEQ ID NO: 76) | | | | | | | |
| PCBP1 | SIQGQHTISPLDLAKLNQV | R21(Cit.) | MHC-II | | | | | |
| | AR*QQSHF (SEQ ID NO: 77) | | | | | | | |
| PCSK5 | AEKWTTVPR*QHVCVEST | R9(Cit.); | MHC-II | | | | | |
| | DR*QIKTIRPN (SEQ ID | R19(Cit.) | | | | | | |
| | NO: 78) | | | | | | | |
| PEX7 | LLVTGAVDCSLRGWDLR* | R17(Cit.) | MHC-II | | | | | |
| | N (SEQ ID NO: 79) | | | | | | | |
| PFN1 | R*TKSTGGAPTENVTVTKT | R1(Cit.) | MHC-II | MHC-II | | | | |
| | DKTL (SEQ ID NO: 80) | | | | | | | |
| PGK1 | HLGR*PDGVPMPDKYSLE | R4(Cit.) | MHC-II | | | | | |
| | PVAVELK (SEQ ID NO: 81) | | | | | | | |
| PGLS | LAR*ELPAAVAPAGPASLA | R3(Cit.); | MHC-II | | HLA- | 473.3 | 1.1 | Strong |
| | R*WTLG (SEQ ID NO: 82) | R19(Cit.) | | | DPA10103- | | | |
| | | | | | DPB10301 | | | |
| | | | | | DRB1_0101 | 48.4 | 7 | Moderate |
| PGLS | LARELPAAVAPAGPASLA | R19(Cit.) | MHC-II | | | | | |
| | R*WTLG (SEQ ID NO: 83) | | | | | | | |
| PGLS | RLVPFDHAESTYGLYR*TH | R16(Cit.) | MHC-II | | | | | |
| | L (SEQ ID NO: 84) | | | | | | | |
| PHGDH | QVVGR*AGTGVDNVDL | R5(Cit.) | MHC-II | | | | | |
| | (SEQ ID NO: 85) | | | | | | | |
| PLIN3 | DVASVQQQR*QEQSY | R9(Cit.) | | MHC-II | | | | |
| | (SEQ ID NO: 86) | | | | | | | |
| PPIA | GGDFTR*HNGTGGKSIYGE | R6(Cit.) | MHC-II | | | | | |
| | KFEDENF (SEQ ID NO: 87) | | | | | | | |
| PPIA | GGDFTR*HNGTGGKSIYGE | R6(Cit.) | MHC-II | | | | | |
| | KFEDEN (SEQ ID NO: 88) | | | | | | | |
| PPIA | GGDFTR*HNGTGGKSIYGE | R6(Cit.) | MHC-II | | | | | |
| | KFEDE (SEQ ID NO: 89) | | | | | | | |
| PPIA | FTR*HNGTGGKSIYGEKF | R3(Cit.) | MHC-II | | | | | |
| | (SEQ ID NO: 90) | | | | | | | |
| PPIAL4E | ENLIR*KHTGSGILSMANA | R5(Cit.) | MHC-II | | | | | |
| | GPNINGSQF (SEQ ID | | | | | | | |
| | NO: 91) | | | | | | | |
| PPIAL4E | LIR*KHTGSGILSMANAGP | R3(Cit.); | | MHC-II | | | | |
| | NTN (SEQ ID NO: 92) | M13(Oxi.) | | | | | | |
| PPIAL4E | LIR*KHTGSGILSMANAGP | R3(Cit.); | MHC-II | | | | | |
| | NINGSQFF (SEQ ID NO: 93) | M13(Oxi.) | | | | | | |
| PPIAL4E | LIR*KHTGSGILSMANAGP | R3(Cit.); | MHC-II | | | | | |
| | NINGSQF (SEQ ID NO: 94) | M13(Oxi.) | | | | | | |
| PPIAL4E | LIR*KHTGSGILSMANAGP | R3(Cit.) | MHC-II | MHC-II | | | | |
| | NTN (SEQ ID NO: 95) | | | | | | | |
| PPIAL4E | LIR*KHTGSGILSMANAGP | R3(Cit.) | MHC-II | | | | | |
| | N (SEQ ID NO: 96) | | | | | | | |
| PPIAL4E | LIR*KHTGSGILSMANAGP | R3(Cit.) | MHC-II | | | | | |
| | NINGSQF (SEQ ID NO: 97) | | | | | | | |
| PPIAL4E | LIR*KHTGSGILSMANAGP | R3(Cit.) | MHC-II | | | | | |
| | NINGSQFF (SEQ ID NO: 98) | | | | | | | |
| PPP1R2P3 | LETADGESMNTEESNQGS | R29(Cit.) | MHC-II | | | | | |
| | TPSDQQQNKLR* (SEQ ID | | | | | | | |
| | NO: 99) | | | | | | | |
| PPPIR2P3 | LETADGESMNTEESNQGS | R29(Cit.) | MHC-II | | | | | |
| | TPSDQQQNKLR*SS (SEQ | | | | | | | |
| | ID NO: 100) | | | | | | | |
| PPPIR7 | WSDLDELKGAR*SLET | R11(Cit.) | MHC-II | | DRB1_0101 | 70.6 | 9.5 | Moderate |
| | (SEQ ID NO: 101) | | | | | | | |
| PRDX1 | HIDDKGILR*QITVNDLPVG | R9(Cit.) | MHC-II | | HLA- | 4343.5 | 9 | Moderate |
| | RSVDET (SEQ ID NO: 102) | | | | DPA10103- | | | |
| | | | | | DPB10301 | | | |
| PRDX1 | VNTPKKQGGLGPMNIPLV | R23(Cit.) | MHC-II | | | | | |
| | SDPKR*TIAQDYG (SEQ ID | | | | | | | |
| | NO: 103) | | | | | | | |
| PRDX2 | IIDGKGVLR*QITVNDLPV | R9(Cit.) | MHC-II | | | | | |
| | GRSVDEAL (SEQ ID | | | | | | | |
| | NO: 104) | | | | | | | |
| PRDX2 | R*R*LSEDYGVLKTDEGIA | R1(Cit.); | MHC-II | | DRB5_0101 | 346.5 | 9.5 | Moderate |
| | YRG (SEQ ID NO: 105) | R2(Cit.) | | | | | | |
| PSMA5 | QFGEEDADPGAMSR*PFG | R14(Cit.) | MHC-II | | | | | |
| | VAL (SEQ ID NO: 106) | | | | | | | |
| RAD23A | GQENPQLLQQISR*HQEQF | R13(Cit.) | MHC-II | | | | | |
| | (SEQ ID NO: 107) | | | | | | | |
| RAD23B | LQQIGR*ENPQLLQQISQH | R6(Cit.) | MHC-II | | | | | |
| | QEH (SEQ ID NO: 108) | | | | | | | |
| RPS21 | NVAEVDKVTGR*FNGQFK | R11(Cit.) | MHC-II | | | | | |
| | TY (SEQ ID NO: 109) | | | | | | | |
| RPS25 | LSKGLIKLVSKHR*AQVIY | R13(Cit.) | | MHC-II | DRB5_0101 | 4.3 | 0.05 | Strong |
| | TRNTKGGDAPAAGEDA | | | | | | | |
| | (SEQ ID NO: 110) | | | | | | | |
| | | | | | DRB5_0101 | 5.2 | 0.08 | Strong |
| | | | | | DRB1_0101 | 48.9 | 7 | Moderate |
| | | | | | HLA- | 3255.9 | 6.5 | Moderate |
| | | | | | DPA10103- | | | |
| | | | | | DPB10301 | | | |
| RPS25 | KGLIKLVSKHR*AQVIYTR | R11(Cit.) | MHC-II | | | | | |
| | NTKGGDAPAAGEDA (SEQ | | | | | | | |
| | ID NO: 111) | | | | | | | |
| S100A6 | MEDLDR*NKDQEVNFQE | R6(Cit.) | MHC-II | | | | | |
| | (SEQ ID NO: 112) | | | | | | | |
| S100A6 | MEDLDR*NKDQEVNF | M1(Oxi.); | MHC-II | | | | | |
| | (SEQ ID NO: 113) | R6(Cit.) | | | | | | |
| S100A9 | MAR*LTWASHEKMHEGD | R3(Cit.) | MHC-II | | | | | |
| | EGPGHHHKPGLGEG (SEQ | | | | | | | |
| | ID NO: 114) | | | | | | | |
| S100A9 | MAR*LTWASHEKMHEGD | R3(Cit.) | MHC-II | | | | | |
| | EGPGHHHKPGLGEGTP | | | | | | | |
| | (SEQ ID NO: 115) | | | | | | | |
| S100A9 | MAR*LTWASHEKMHEGD | M1(Oxi.); | MHC-II | | | | | |
| | EGPGHHHKPGLGEGTP | R3(Cit.) | | | | | | |
| | (SEQ ID NO: 116) | | | | | | | |
| SERBP1 | PGHLQEGFGCVVTNR*F | R15(Cit.) | MHC-II | | | | | |
| | (SEQ ID NO: 117) | | | | | | | |
| SERBP1 | PGHLQEGFGCVVTNR*FD | R15(Cit.) | MHC-II | | | | | |
| | QL (SEQ ID NO: 118) | | | | | | | |
| SFN | GLLDSHLIKEAGDAESR* | R17(Cit.) | MHC-II | | | | | |
| | (SEQ ID NO: 119) | | | | | | | |
| SLC9A3R1 | LR*AQEAPGQAEPPAAAE | R2(Cit.) | MHC-II | | | | | |
| | VQGAGN (SEQ ID NO: 120) | | | | | | | |
| SRF | GGTR*GANGGR*VPGNGA | R4(Cit.); | MHC-II | | | | | |
| | GLGPGR* (SEQ ID NO: 121) | R10(Cit.); | | | | | | |
| | | R22(Cit.) | | | | | | |
| SRP14 | LR*ATDGKKKISTVVSSKE | R2(Cit.) | MHC-II | | DRB5_0101 | 259.3 | 8 | Moderate |
| | VNKFQMA (SEQ ID | | | | | | | |
| | NO: 122) | | | | | | | |
| | | | | | HLA- | 4255.9 | 8.5 | Moderate |
| | | | | | DPA10103- | | | |
| | | | | | DPB10301 | | | |
| SRP9 | MVAKEAR*NVTMETE | R7(Cit.) | | | | | | |
| | (SEQ ID NO: 123) | | | | | | | |
| TAGLN2 | LGGLAVAR*DDGL (SEQ | R8(Cit.) | | MHC-II | | | | |
| | ID NO: 124) | | | | | | | |
| TAGLN2 | VIGLQMGTNRGASQAGMT | R24(Cit.) | MHC-II | | | | | |
| | GYGMPR*Q (SEQ ID | | | | | | | |
| | NO: 125) | | | | | | | |
| TAGLN2 | TNRGASQAGMTGYGMPR | R17(Cit.) | MHC-II | MHC-II | | | | |
| | *QIL (SEQ ID NO: 126) | | | | | | | |
| TAGLN2 | QLQEGKNVIGLQMGTNR* | R17(Cit.) | MHC-II | | | | | |
| | GASQAGMTGYGMPRQIL | | | | | | | |
| | (SEQ ID NO: 127) | | | | | | | |
| TAGLN2 | VIGLQMGTNR*GASQAGM | R10(Cit.); | MHC-II | | | | | |
| | TGYGMPRQIL (SEQ ID | M17(Oxi.) | | | | | | |
| | NO: 128) | | | | | | | |
| TECR | FTKTHPQWYPAR*QSL | R12(Cit.) | MHC-II | | | | | |
| | (SEQ ID NO: 129) | | | | | | | |
| TIMM9 | HIQQNEALAAKAGLLGQP | R19(Cit.) | MHC-II | | HLA- | 2249.1 | 4.5 | Moderate |
| | R* (SEQ ID NO: 130) | | | | DPA10103- | | | |
| | | | | | DPB10301 | | | |
| TIPRL | IFPER*IDPNPADSQKSTQV | R5(Cit.) | MHC-II | | | | | |
| | E (SEQ ID NO: 131) | | | | | | | |
| UBA7 | R*AEASQELLAQ (SEQ ID | R1(Cit.) | | MHC-I | | | | |
| | NO: 132) | | | | | | | |
| UBE2L3 | WQGLIVPDNPPYDKGAFR | R18(Cit.) | MHC-II | | | | | |
| | * (SEQ ID NO: 133) | | | | | | | |
| YWHAZ | KSVTEQGAELSNEER*NLL | R15(Cit.) | MHC-II | | | | | |
| | (SEQ ID NO: 134) | | | | | | | |
| ZBED1 | MLVSNR*VSWWGSTLAM | M1(Oxi.); | MHC-II | | | | | |
| | LQRLKEQQFVIAGVL (SEQ | R6(Cit.) | | | | | | |
| | ID NO: 135) | | | | | | | |

Oxi.: oxidized Met. (represented as M)
Cit.: citrullinated Arg. (represented as R*)
#Affinity prediction was based on unmodified sequence.

### Example 4. Positive Correlation of PADI2 and Citrullinome in Tissue Microarray Analysis

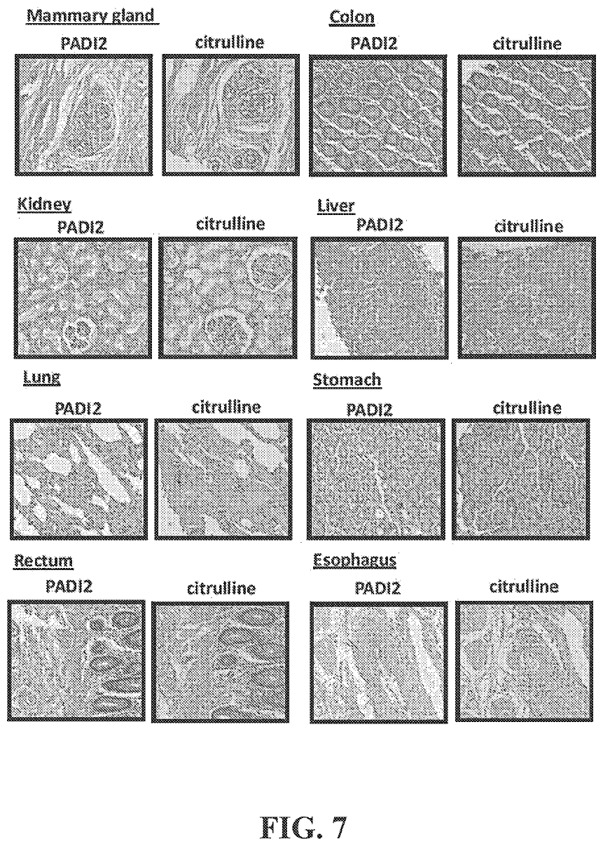

To confirm the association of PADI2 expression with protein citrullination in breast tumor tissues, we assessed PADI2 protein expression and citrullinome by means of immunohistochemistry (IHC) in 422 breast tumors (Table 3). PADI2 protein expression in the cohort of 422 breast tumors was statistically significantly higher in Grade III vs Grades I and 11 tumors (X²-square test, 2-sided P<0.0001), estrogen receptor (ER)−(HR-/HER2-enriched and TNBC) versus ER+(Luminal AB) (X²-square test, 2-sided P<0.0001) and TNBC versus Non-TNBC (X²-square test, 2 sided P<0.0001) (Table 3). PADI2 expression was not associated with age or stage in a statistically significant manner (Table 3). Staining for peptidyl-citrulline (Low+Low-Moderate vs Moderate-High+High) was significantly positively correlated with PADI2 protein expression (OR 4.45, 95% CI: 2.48-7.78; X²-square test, 2-sided P<0.0001) (Table 3, data not shown). Staining for PADI2 and peptidyl-citrulline was negative in mammary gland, colon, kidney, liver, lung, stomach, rectum and esophagus as well as the tumor's adjacent normal tissue (FIG. 7).

TABLE 3
Citrullinated protein count in breast cancer tissues (n = 422) in relation to PADI2
expression and clinical parameters.

| | PADI2 expression* | | | | | | |
|---|---|---|---|---|---|---|---|
| | | Low- | Moderate- | | | P | OR |
| Variable | Low | moderate | high | High | Comparison | value† | (95%CI)‡ |
| Age | | | | | | | |
| ≤50 | 77 | 57 | 40 | 60 | Age ≤50 vs >50 | 0.0477 | 1.55 (0.91 to |
| | | | | | | | 2.59) |
| >50 | 43 | 45 | 48 | 52 | | | |
| Grade | | | | | | | |
| I-II | 93 | 68 | 56 | 50 | Grade I + II vs III | <0.0001 | 8.12 (3.83 to |
| | | | | | | | 17.44) |
| III | 11 | 21 | 22 | 48 | | | |
| Unknown | 16 | 13 | 10 | 14 | | | |
| Stage | | | | | | | |
| I | 12 | 11 | 10 | 10 | Stage I vs | 0.8284 | 1.13 (0.49 to |
| | | | | | II+III+IV | | 2.75) |
| II | 90 | 75 | 62 | 85 | Stage I + II vs | 0.8618 | 1.01 (0.51to |
| | | | | | III + IV | | 2.01) |
| III | 17 | 15 | 16 | 17 | | | |
| IV | 1 | 1 | 0 | 0 | | | |
| Receptor | | | | | | | |
| Luminal A | 61 | 30 | 19 | 17 | HR+ vs HR− | <0.0001 | 6.71 (3.75 to |
| | | | | | | | 11.66) |
| Luminal B | 32 | 29 | 17 | 21 | TNBC vs non-TNBC | <0.0001 | 4.33 (2.41 to |
| | | | | | | | 8.12) |
| HER2- | 7 | 14 | 14 | 22 | | | |
| enriched | | | | | | | |
| TNBC | 20 | 29 | 38 | 52 | | | |
| Citrullination | | | | | | | |
| Low | 19 | 9 | 9 | 11 | Citrullination | <0.0001 | 4.45 (2.48 to |
| | | | | | (low+ low-moderate | | 7.78) |
| | | | | | vs moderate-high + | | |
| | | | | | high) | | |
| Low-moderate | 77 | 57 | 49 | 42 | Citrullination | <0.0001 | 7.97 (2.5 to |
| | | | | | (high vs rest) | | 25.95) |
| Moderate-high | 21 | 29 | 22 | 40 | | | |
| High | 3 | 7 | 8 | 19 | | | |

*PADI2 and peptidyl-citrulline (citrullination) staining positivity: low: 0%-24%; low- moderate: 25%-49%; moderate-high: 50%-74%; high: ≥75%.
†P value represents two-sided X2 test for trend between PADI2 staining and the respective comparative groups.
‡OR (95% CI) for comparison of high PADI2 (≥75% staining positivity) vs low PADI2 (0%-24% staining positivity) expression.
HR, Hormone Receptor; PADI2, protein arginine deiminase 2; TNBC, Triple Negative Breast Cancer.

### Example 5. PADI2 is Associated with Tumor Immunophenotype

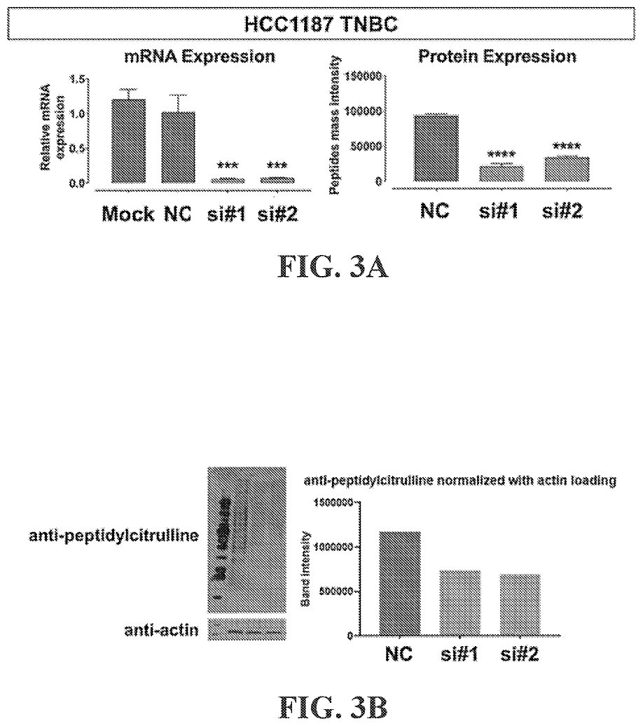

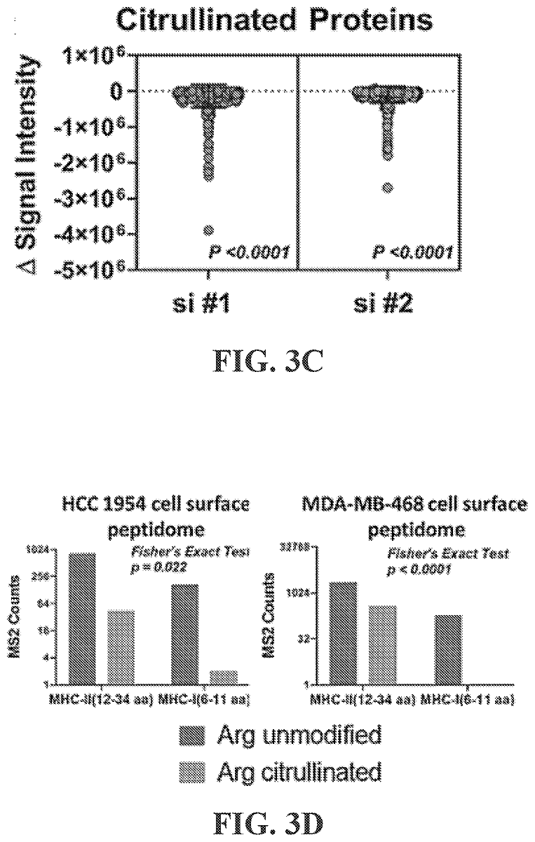

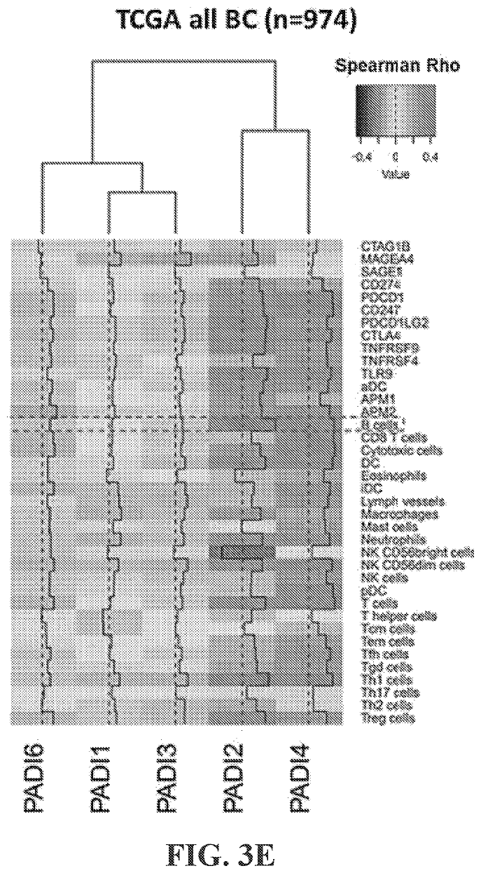

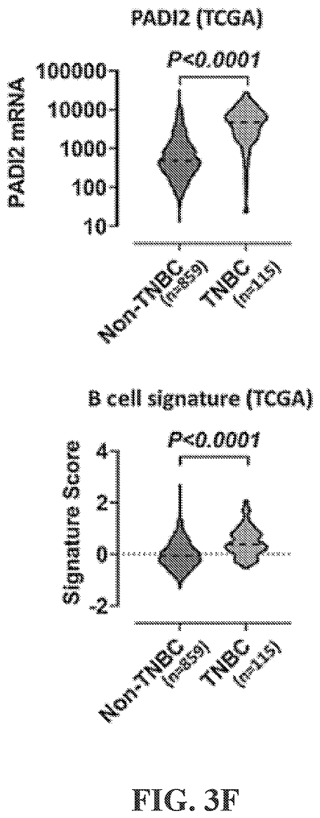

We determined whether PADI2 is a major contributor to protein arginine deamination by knocking down PADI2 with siRNA in the TNBC HCC1187 cell line and performing global assessment of protein citrullination by immunoblots as well as by mass spectrometry analysis. Knockdown of PADI2 reduced PADI2 mRNA and protein (FIG. 3A). Immunoblots demonstrated reductions in protein citrullination globally (FIG. 31B). Mass spectrometry-based analyses similarly demonstrated that citrullinated digested peptides were statistically significantly reduced (paired 2-sided t-test, p<0.0001) compared with control (FIG. 3Q). We next evaluated the cell surface HLA bound peptidome of HR-MHER2-enriched (HCC 1954) and TNBC (MDA-MB-468) cell lines as a means to evaluate MH-C antigen presentation (ref. 26). Specifically, we screened for the presence of citrullinated peptides that were putatively in the MHC class II binding peptide length (12-34 amino acids) or in the MHC class I binding peptide length (6-11 amino acids) with mild acid elution method that can recover class I (ref. 26) and class 11 (ref. 41). We identified 23 (MHC class II) and 1 (MHC class I) citrullinated peptides in HCC1954 and 126 (MHC class II) and 0 (MHC class I) in MDA-MB-468 (FIG. 3D, Table 2) indicating predominance of citrullinated peptides within the MHC class II binding peptide length, compared with the MHC class I binding peptide length (Fisher's exact test, 2-sided p=0.022 for HCC1954 and p<0.0001 for MDA-MB-468) (FIG. 3D). The citrullinated peptides identified were further searched with the affinity prediction software NetMHC-II pan V.4.1 (refs. 27 and 28) considered as unmodified form, and the immunogenicity of actual citrullinated forms was possibly enhanced compared to the predicted result (Table 2).

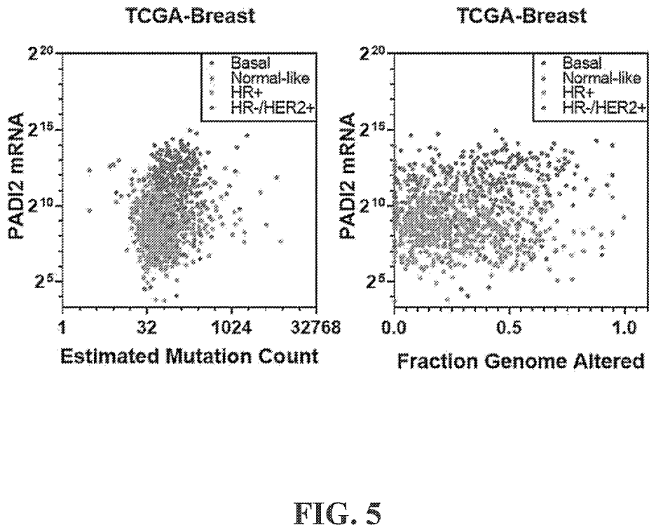

We next evaluated whether elevated PADI2 expression is associated with a distinct tumor immunophenotype. Using TCGA-derived mRNA expression datasets for 974 breast tumors, we first performed Spearman's correlation analyses between tumor PADI2 mRNA expression and gene expression profiles of checkpoint blockade-related genes as well as gene expression signatures reflective of immune cell infiltrates (ref 34). Statistically significant positive correlations were observed between PADI2 mRNA and virtually all checkpoint blockade-related genes as well as gene expression signatures reflective of immune cell infiltrates (FIG. 3E, Table 4). PADI2 mRNA expression was most positively correlated with a gene signature for B cell infiltrates (p<0.0001, FIG. 3E, Spearman's r=0.49 (0.44-0.53), Table 4). TNBC is considered to be more immunogenic than non-TNBC because of genomic instability and higher rates of mutation (ref 42). We previously reported that TNBC exhibited enhanced immune cell infiltrates compared with non-TNBC tumors (ref. 33). The higher expression of PADI2 in TNBC was associated with an enhanced B cell response (p<0.0001) in comparison with non-TNBC (Luminal A/B and HR-/HER2 enriched combined) (FIG. 3F; FIG. 5 and Table 5). Notably, the association between mutational burden (defined herein as the number of mutation events per case) with mRNA expression of PADI2 (Spearman's r=0.10 (95% CI: −0.05 to 0.26); p=0.17) or B-cell gene-based signatures (Spearman's r=−0.10 (95% CI: −0.25 to 0.06); p=0.20) was non-significant in basal-type TNBC tumors (FIG. 5 and Table 5). These findings implicate that the association between PADI2 and B cell gene signatures is independent of mutational burden.

TABLE 4
TCGA breast cancer PADI2 and immune
gene signature correlation.

| | Spearman | | |
|---|---|---|---|
| Variable | Coefficient | 95% CI | p-value |
| | PADI2-All BrCa Cases | | |
| CTAG1B | 0.1704 | 0.1072 to 0.2323 | <0.0001 |
| MAGEA4 | 0.245 | 0.1835 to 0.3047 | <0.0001 |
| SAGE1 | 0.03806 | −0.02644 to 0.1022 | 0.2336 |
| CD274 | 0.2517 | 0.1904 to 0.3111 | <0.0001 |
| PDCD1 | 0.2967 | 0.2368 to 0.3544 | <0.0001 |
| CD247 | 0.3139 | 0.2546 to 0.3708 | <0.0001 |
| PDCD1LG2 | 0.3675 | 0.3104 to 0.4219 | <0.0001 |
| CTLA4 | 0.3411 | 0.2829 to 0.3968 | <0.0001 |
| TNFRSF9 | 0.3346 | 0.2761 to 0.3906 | <0.0001 |
| TNFRSF4 | 0.1612 | 0.09782 to 0.2234 | <0.0001 |
| TLR9 | 0.3782 | 0.3216 to 0.4321 | <0.0001 |
| aDC | 0.3114 | 0.2520 to 0.3685 | <0.0001 |
| APM1 | 0.2642 | 0.2032 to 0.3232 | <0.0001 |
| APM2 | 0.2918 | 0.2318 to 0.3497 | <0.0001 |
| B cells | 0.486 | 0.4352 to 0.5337 | <0.0001 |
| CD8 T cells | 0.1252 | 0.06123 to 0.1881 | <0.0001 |
| Cytotoxic cells | 0.2694 | 0.2085 to 0.3281 | <0.0001 |
| DC | 0.3383 | 0.2800 to 0.3942 | <0.0001 |
| Eosinophils | −0.1217 | −0.1847 to −0.05776 | <0.0001 |
| iDC | 0.1669 | 0.1035 to 0.2288 | <0.0001 |
| Lymph vessels | 0.1338 | 0.06994 to 0.1965 | <0.0001 |
| Macrophages | 0.2762 | 0.2156 to 0.3347 | <0.0001 |
| Mast cells | −0.01705 | −0.08140 to 0.04743 | 0.5937 |
| Neutrophils | 0.3029 | 0.2432 to 0.3603 | <0.0001 |
| NK CD56bright cells | −0.3105 | −0.3676 to −0.2511 | <0.0001 |
| NK CD56dim cells | 0.3071 | 0.2476 to 0.3643 | <0.0001 |
| NK cells | 0.1169 | 0.05284 to 0.1800 | 0.0002 |
| pDC | 0.1232 | 0.05924 to 0.1862 | 0.0001 |
| T cells | 0.3392 | 0.2810 to 0.3950 | <0.0001 |
| T helper cells | 0.1393 | 0.07557 to 0.2020 | <0.0001 |
| Tcm cells | 0.02992 | −0.03458 to 0.09418 | 0.3491 |
| Tem cells | 0.1854 | 0.1224 to 0.2469 | <0.0001 |
| Tfh cells | 0.1892 | 0.1263 to 0.2505 | <0.0001 |
| Tgd cells | 0.221 | 0.1588 to 0.2814 | <0.0001 |
| Th1 cells | 0.3935 | 0.3376 to 0.4466 | <0.0001 |
| Th17 cells | 0.06732 | 0.002898 to 0.1312 | 0.035 |
| Th2 cells | 0.2154 | 0.1530 to 0.2760 | <0.0001 |
| Treg cells | 0.3411 | 0.2829 to 0.3968 | <0.0001 |
| | PADI2-HR+ Subtype | | |
| CTAG1B | 0.1246 | 0.04860 to 0.1992 | 0.001 |
| MAGEA4 | 0.08475 | 0.008303 to 0.1602 | 0.0254 |
| SAGE1 | 0.04367 | −0.03295 to 0.1198 | 0.2499 |
| CD274 | 0.2083 | 0.1339 to 0.2803 | <0.0001 |
| PDCD1 | 0.2792 | 0.2071 to 0.3483 | <0.0001 |
| CD247 | 0.3044 | 0.2333 to 0.3722 | <0.0001 |
| PDCD1LG2 | 0.3125 | 0.2418 to 0.3800 | <0.0001 |
| CTLA4 | 0.2688 | 0.1963 to 0.3383 | <0.0001 |
| TNFRSF9 | 0.2568 | 0.1839 to 0.3269 | <0.0001 |
| TNFRSF4 | 0.1028 | 0.02651 to 0.1779 | 0.0066 |
| TLR9 | 0.2685 | 0.1960 to 0.3380 | <0.0001 |
| aDC | 0.2119 | 0.1377 to 0.2838 | <0.0001 |
| APM1 | 0.1085 | 0.03228 to 0.1835 | 0.0042 |
| APM2 | 0.3795 | 0.3121 to 0.4432 | <0.0001 |
| B cells | 0.4207 | 0.3556 to 0.4817 | <0.0001 |
| CD8 T cells | 0.3185 | 0.2480 to 0.3856 | <0.0001 |
| Cytotoxic cells | 0.3399 | 0.2704 to 0.4059 | <0.0001 |
| DC | 0.3592 | 0.2907 to 0.4240 | <0.0001 |
| Eosinophils | 0.1901 | 0.1153 to 0.2628 | <0.0001 |
| iDC | 0.3555 | 0.2868 to 0.4206 | <0.0001 |
| Lymph vessels | 0.3399 | 0.2705 to 0.4059 | <0.0001 |
| Macrophages | 0.3057 | 0.2347 to 0.3735 | <0.0001 |
| Mast cells | 0.3276 | 0.2575 to 0.3942 | <0.0001 |
| Neutrophils | 0.3796 | 0.3121 to 0.4432 | <0.0001 |
| NK CD56bright cells | −0.06905 | −0.1448 to 0.007496 | 0.0687 |
| NK CD56dim cells | 0.3367 | 0.2671 to 0.4028 | <0.0001 |
| NK cells | 0.3522 | 0.2833 to 0.4174 | <0.0001 |
| pDC | 0.1882 | 0.1133 to 0.2609 | <0.0001 |
| T cells | 0.3668 | 0.2986 to 0.4312 | <0.0001 |
| T helper cells | 0.02243 | −0.05416 to 0.09877 | 0.5546 |
| Tcm cells | 0.1575 | 0.08197 to 0.2312 | <0.0001 |
| Tem cells | 0.1771 | 0.1020 to 0.2502 | <0.0001 |
| Tfh cells | 0.3347 | 0.2649 to 0.4009 | <0.0001 |
| Tgd cells | 0.2918 | 0.2202 to 0.3603 | <0.0001 |
| Th1 cells | 0.3608 | 0.2924 to 0.4256 | <0.0001 |
| Th17 cells | 0.09174 | 0.01535 to 0.1671 | 0.0155 |
| Th2 cells | 0.1035 | 0.02717 to 0.1786 | 0.0063 |
| Treg cells | 0.2707 | 0.1983 to 0.3402 | <0.0001 |
| | PADI2-ER−/Her2+ Subtype | | |
| CTAG1B | 0.01529 | −0.2143 to 0.2433 | 0.8943 |
| MAGEA4 | 0.1571 | −0.07444 to 0.3726 | 0.1695 |
| SAGE1 | −0.07675 | −0.3004 to 0.1549 | 0.5042 |
| CD274 | 0.1726 | −0.05860 to 0.3862 | 0.1308 |
| PDCD1 | −0.0246 | −0.2521 to 0.2054 | 0.8307 |
| CD247 | 0.07358 | −0.1580 to 0.2975 | 0.522 |
| PDCD1LG2 | 0.06453 | −0.1668 to 0.2891 | 0.5746 |
| CTLA4 | 0.02055 | −0.2093 to 0.2483 | 0.8583 |
| TNFRSF9 | 0.09164 | −0.1402 to 0.3139 | 0.4249 |
| TNFRSF4 | −0.1274 | −0.3462 to 0.1045 | 0.2662 |
| TLR9 | 0.09926 | −0.1326 to 0.3208 | 0.3873 |
| aDC | 0.07836 | −0.1533 to 0.3018 | 0.4953 |
| APM1 | −0.1234 | −0.3426 to 0.1085 | 0.2816 |
| APM2 | 0.05323 | −0.1778 to 0.2787 | 0.6435 |
| B cells | 0.1194 | −0.1125 to 0.3390 | 0.2976 |
| CD8 T cells | −0.1327 | −0.3509 to 0.09918 | 0.2467 |
| Cytotoxic cells | 0.01734 | −0.2124 to 0.2452 | 0.8802 |
| DC | −0.0247 | −0.2522 to 0.2053 | 0.8301 |
| Eosinophils | −0.08739 | −0.3101 to 0.1444 | 0.4468 |
| iDC | −0.007119 | −0.2356 to 0.2221 | 0.9507 |
| Lymph vessels | −0.153 | −0.3690 to 0.07858 | 0.181 |
| Macrophages | −0.03674 | −0.2634 to 0.1938 | 0.7495 |
| Mast cells | −0.1678 | −0.3820 to 0.06347 | 0.1419 |
| Neutrophils | 0.008763 | −0.2206 to 0.2372 | 0.9393 |
| NK CD56bright cells | −0.06645 | −0.2909 to 0.1649 | 0.5632 |
| NK CD56dim cells | −0.0487 | −0.2745 to 0.1822 | 0.672 |
| NK cells | −0.2642 | −0.4649 to −0.03754 | 0.0194 |
| pDC | 0.03975 | −0.1909 to 0.2662 | 0.7297 |
| T cells | 0.07293 | −0.1586 to 0.2969 | 0.5257 |
| T helper cells | 0.113 | −0.1189 to 0.3333 | 0.3245 |
| Tcm cells | −0.1296 | −0.3481 to 0.1023 | 0.2582 |
| Tem cells | −0.01463 | −0.2427 to 0.2150 | 0.8988 |
| Tfh cells | −0.01703 | −0.2450 to 0.2127 | 0.8823 |
| Tgd cells | 0.02561 | −0.2045 to 0.2530 | 0.8239 |
| Th1 cells | 0.02227 | −0.2077 to 0.2499 | 0.8466 |
| Th17 cells | −0.02159 | −0.2492 to 0.2083 | 0.8512 |
| Th2 cells | 0.0376 | −0.1929 to 0.2642 | 0.7438 |
| Treg cells | 0.1085 | −0.1235 to 0.3292 | 0.3444 |
| | PADI2-TNBC (Basal) Subtype | | |
| CTAG1B | 0.061 | −0.09396 to 0.2138 | 0.425 |
| MAGEA4 | 0.067 | −0.08793 to 0.2196 | 0.381 |
| SAGE1 | 0.037 | −0.1178 to 0.1906 | 0.628 |
| CD274 | 0.124 | −0.03114 to 0.2731 | 0.106 |
| PDCD1 | 0.069 | −0.08621 to 0.2212 | 0.369 |
| CD247 | 0.071 | −0.08394 to 0.2234 | 0.353 |
| PDCD1LG2 | 0.206 | 0.05357 to 0.3496 | 0.007 |
| CTLA4 | 0.055 | −0.1005 to 0.2075 | 0.476 |
| TNFRSF9 | 0.196 | 0.04248 to 0.3398 | 0.01 |
| TNFRSF4 | 0.001 | −0.1533 to 0.1556 | 0.988 |
| TLR9 | 0.026 | −0.1286 to 0.1800 | 0.733 |
| aDC | 0.098 | −0.05704 to 0.2489 | 0.201 |
| APM1 | 0.059 | −0.09588 to 0.2119 | 0.440 |
| APM2 | 0.075 | −0.08026 to 0.2269 | 0.329 |
| B cells | 0.186 | 0.03233 to 0.3308 | 0.015 |
| CD8 T cells | −0.072 | −0.2236 to 0.08375 | 0.352 |
| Cytotoxic cells | 0.075 | −0.08084 to 0.2264 | 0.333 |
| DC | 0.085 | −0.07074 to 0.2360 | 0.271 |
| Eosinophils | 0.092 | −0.06369 to 0.2427 | 0.233 |
| iDC | 0.018 | −0.1364 to 0.1724 | 0.811 |
| Lymph vessels | 0.003 | −0.1519 to 0.1570 | 0.973 |
| Macrophages | 0.088 | −0.06765 to 0.2389 | 0.254 |
| Mast cells | 0.027 | −0.1277 to 0.1810 | 0.723 |
| Neutrophils | 0.07 | −0.08540 to 0.2220 | 0.363 |
| NK CD56bright cells | −0.138 | −0.2866 to 0.01646 | 0.071 |
| NK CD56dim cells | −0.04 | −0.1930 to 0.1154 | 0.606 |
| NK cells | −0.034 | −0.1876 to 0.1209 | 0.657 |
| pDC | 0.002 | −0.1521 to 0.1568 | 0.975 |
| T cells | 0.136 | −0.01861 to 0.2847 | 0.076 |
| T helper cells | 0.092 | −0.06290 to 0.2434 | 0.229 |
| Tcm cells | 0.124 | −0.03085 to 0.2734 | 0.106 |
| Tem cells | 0.232 | 0.08086 to 0.3734 | 0.002 |
| Tfh cells | 0.12 | −0.03499 to 0.2695 | 0.118 |
| Tgd cells | −0.1 | −0.2506 to 0.05524 | 0.193 |
| Th1 cells | 0.131 | −0.02373 to 0.2800 | 0.087 |
| Th17 cells | 0.139 | −0.01551 to 0.2875 | 0.069 |
| Th2 cells | 0.047 | −0.1081 to 0.2001 | 0.540 |
| Treg cells | 0.155 | 0.001025 to 0.3026 | 0.042 |
| | PADI2-Normal like Subtype | | |
| CTAG1B | 0.1842 | −0.1635 to 0.4912 | 0.2822 |
| MAGEA4 | 0.06258 | −0.2809 to 0.3918 | 0.7169 |
| SAGE1 | 0.2133 | −0.1338 to 0.5138 | 0.2116 |
| CD274 | −0.2136 | −0.5141 to 0.1335 | 0.2109 |
| PDCD1 | −0.3761 | −0.6332 to −0.04416 | 0.0238 |
| CD247 | −0.1843 | −0.4912 to 0.1634 | 0.2819 |
| PDCD1LG2 | −0.2288 | −0.5257 to 0.1178 | 0.1795 |
| CTLA4 | −0.3562 | −0.6193 to −0.02130 | 0.033 |
| TNFRSF9 | −0.1292 | −0.4472 to 0.2178 | 0.4526 |
| TNFRSF4 | −0.3737 | −0.6316 to −0.04147 | 0.0247 |
| TLR9 | −0.07207 | −0.3998 to 0.2720 | 0.6762 |
| aDC | −0.312 | −0.5876 to 0.02854 | 0.064 |
| APM1 | −0.2468 | −0.5394 to 0.09890 | 0.1467 |
| APM2 | −0.1846 | −0.4914 to 0.1631 | 0.2812 |
| B cells | 0.182 | −0.1657 to 0.4894 | 0.2881 |
| CD8 T cells | −0.3264 | −0.5980 to 0.01250 | 0.052 |
| Cytotoxic cells | −0.2252 | −0.5230 to 0.1215 | 0.1866 |
| DC | −0.2427 | −0.5363 to 0.1032 | 0.1538 |
| Eosinophils | −0.1395 | −0.4556 to 0.2078 | 0.4171 |
| iDC | −0.2775 | −0.5623 to 0.06623 | 0.1013 |
| Lymph vessels | −0.1297 | −0.4476 to 0.2173 | 0.4508 |
| Macrophages | −0.1681 | −0.4784 to 0.1796 | 0.3271 |
| Mast cells | −0.1851 | −0.4919 to 0.1626 | 0.2799 |
| Neutrophils | −0.2607 | −0.5499 to 0.08416 | 0.1245 |
| NK CD56bright cells | −0.294 | −0.5745 to 0.04835 | 0.0818 |
| NK CD56dim cells | −0.3668 | −0.6267 to −0.03343 | 0.0278 |
| NK cells | −0.3346 | −0.6039 to 0.003250 | 0.0461 |
| pDC | −0.1871 | −0.4935 to 0.1605 | 0.2745 |
| T cells | −0.1444 | −0.4595 to 0.2030 | 0.4008 |
| T helper cells | 0.3264 | −0.01250 to 0.5980 | 0.052 |
| Tcm cells | 0.1421 | −0.2053 to 0.4576 | 0.4084 |
| Tem cells | −0.4739 | −0.6995 to −0.1623 | 0.0035 |
| Tfh cells | −0.03552 | −0.3686 to 0.3056 | 0.837 |
| Tgd cells | −0.1951 | −0.4997 to 0.1524 | 0.2541 |
| Th1 cells | −0.201 | −0.5043 to 0.1464 | 0.2397 |
| Th17 cells | 0.425 | 0.1021 to 0.6668 | 0.0098 |
| Th2 cells | −0.1804 | −0.4882 to 0.1673 | 0.2923 |
| Treg cells | −0.1743 | −0.4833 to 0.1734 | 0.3094 |

TABLE 5
Correlation between PADI2 and mutation burden in TOGA breast cancers.

| | Fraction Genome Altered | | | Estimated Mutation Count | | |
|---|---|---|---|---|---|---|
| | Spearman | | | Spearman | | |
| Group | Coefficient | 95% CI | p-value | Coefficient | 95% CI | p-value |
| All | 0.103 | 0.038 to 0.166 | 0.0013 | 0.242 | 0.181 to 0.302 | <0.0001 |
| Basal | 0.092 | −0.063 to 0.243 | 0.2292 | 0.105 | −0.05 to 0.255 | 0.1723 |
| Normal- | −0.114 | −0.434 to 0.233 | 0.5097 | −0.185 | −0.492 to 0.163 | 0.2808 |
| Like | | | | | | |
| HR+ | −0.11 | −0.185 to −0.034 | 0.0037 | 0.029 | −0.048 to 0.105 | 0.4481 |
| HR−/ | 0.124 | −0.108 to 0.343 | 0.2789 | 0.042 | −0.189 to 0.268 | 0.7159 |
| HER2+ | | | | | | |

### Example 6. Citrullinome Contributes to B Cell Tumor Immune Infiltration and Autoantibody Elevation in Breast Cancer

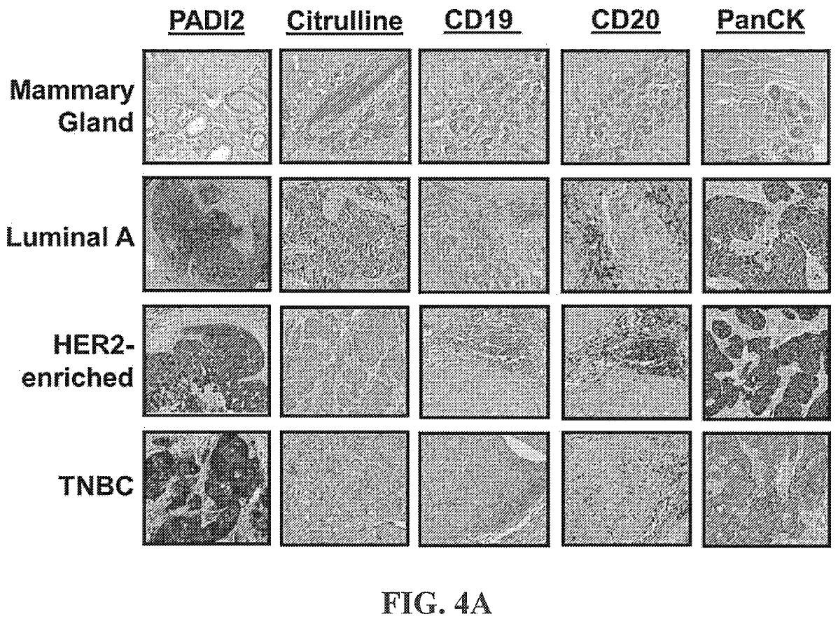

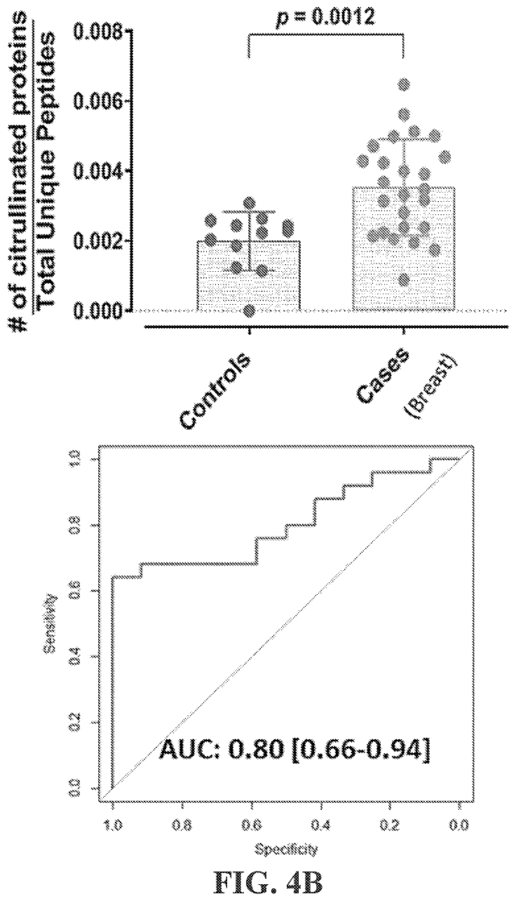

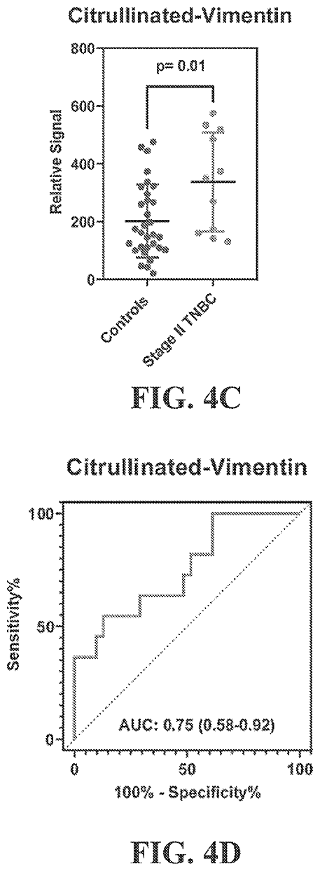

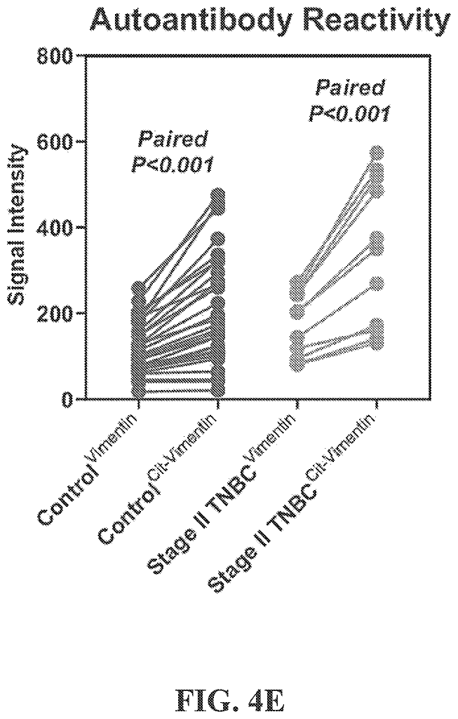

To further confirm the association of PADI2 protein expression with increased tumor protein citrullination and with increased tumor infiltration of B cells, we performed IHC on breast cancer tissue section slides for PADI2, anti-peptidyl-citrulline, Pan-cytokeratin (PanCK) and B cell markers CD19 and CD20 (FIG. 4A). Our findings confirmed that elevated PADI2 and increased protein citrullination were associated with infiltrating B cells (FIG. 4A). We therefore aimed to determine whether a tumor B cell response facilitated by PADI2-mediated citrullination would manifest in elevated circulating plasma autoantibodies. We evaluated IgG bound proteins by mass spectrometry, with 26 plasma pools from 156 patients with breast cancer (10 ER+, 8 HR-/HER2-enriched, 8 TNBC pools) and 11 healthy control pools from 113 cancer-free subjects (Table 6). The number of IgG bound citrullinated proteins was statistically significantly higher in plasmas of breast cancer cases compared with controls (Wilcoxon rank sum test 2-sided, p=0.0012, FIG. 4B, top panel; area under the curve (AUC)=0.80, FIG. 4B, bottom panel). IgG bound citrullinated proteins elevated in patients with breast cancer were further characterized by IPA network analyses. In Luminal A, the top 1 and top 2 networks were cytokeratin complex and estrogen-progesterone centered networks, whereas in TNBC, a cytokeratin complex and MYC centered network that included ENO1 were observed (Table 7 and see FIG. S5 in Katayama et al. 2021). The HR-/Her2-enriched subtype exhibited a cytokeratin complex and an FN1 centered network (Table 7 and see FIG. S5 in Katayama et al. 2021).

TABLE 6
Newly diagnosed breast cancer cohort of the plasma Ig-bound citrullinome.

| | | Sample | Age at diagnosis | |
|---|---|---|---|---|
| Subtype | Stage | pooled | Median | Mean |
| ER/PR+ and HER2− | I | (n = 3/group) | 63 ± 5.9 | 61 ± 5.9 |
| ER/PR+ and HER2− | I | (n = 9/group) | 63 ± 5.3 | 62 ± 5.3 |
| ER/PR+ and HER2− | II | (n = 3/group) | 54 ± 4.7 | 56 ± 4.7 |
| ER/PR+ and HER2− | II | (n = 3/group) | 40 ± 5.9 | 38 ± 5.9 |
| ER/PR+ and HER2− | II | (n = 9/group) | 56 ± 7.9 | 58 ± 7.9 |
| ER/PR+ and HER2− | II | (n = 9/group) | 39 ± 2.9 | 39 ± 2.9 |
| ER/PR+ and HER2− | III | (n = 3/group) | 52 ± 5.8 | 55 ± 5.8 |
| ER/PR+ and HER2− | III | (n = 3/group) | 43 ± 2.5 | 43 ± 2.5 |
| ER/PR+ and HER2− | III | (n = 9/group) | 60 ± 6.0 | 59 ± 6.0 |
| ER/PR+ and HER2− | III | (n = 9/group) | 44 ± 3.8 | 44 ± 3.8 |
| HR−/HER2+ enriched | II | (n = 3/group) | 63 ± 7.6 | 60 ± 7.6 |
| HR−/HER2+ enriched | II | (n = 3/group) | 44 ± 11.0 | 40 ± 11.0 |
| HR−/HER2+ enriched | II | (n = 9/group) | 57 ± 4.0 | 58 ± 4.0 |
| HR−/HER2+ enriched | II | (n = 9/group) | 42 ± 7.3 | 41 ± 7.3 |
| HR−/HER2+ enriched | III | (n = 3/group) | 52 ± 14.3 | 49 ± 14.3 |
| HR−/HER2+ enriched | III | (n = 3/group) | 36 ± 3.8 | 38 ± 3.8 |
| HR−/HER2+ enriched | III | (n = 9/group) | 51 ± 6.6 | 52 ± 6.6 |
| HR−/HER2+ enriched | III | (n = 9/group) | 39 ± 2.5 | 38 ± 2.5 |
| TNBC | II | (n = 3/group) | 60 ± 6.5 | 60 ± 6.5 |
| TNBC | II | (n = 3/group) | 45 ± 6.6 | 46 ± 6.6 |
| TNBC | II | (n = 9/group) | 64 ± 7.4 | 64 ± 7.4 |
| TNBC | II | (n = 9/group) | 42 ± 3.3 | 43 ± 3.3 |
| TNBC | III | (n = 3/group) | 56 ± 8.1 | 52 ± 8.1 |
| TNBC | III | (n = 3/group) | 37 ± 4.5 | 37 ± 4.5 |
| TNBC | III | (n = 9/group) | 58 ± 5.6 | 58 ± 5.6 |
| TNBC | III | (n = 9/group) | 39 ± 7.3 | 39 ± 7.3 |
| Healthy control | | (n = 10/group) | 52 ± 8.4 | 54 ± 8.4 |
| Healthy control | | (n = 10/group) | 44 ± 4.7 | 44 ± 4.7 |
| DCIS (ER/PR+) control | | (n = 10/group) | 58 ± 6.1 | 59 ± 6.1 |
| DCIS (ER/PR+) control | | (n = 10/group) | 43 ± 6.2 | 43 ± 6.2 |
| Healthy control | | (n = 10/group) | 70 ± 2.5 | 70 ± 2.5 |
| Healthy control | | (n = 20/group) | 56 ± 8.7 | 59 ± 8.7 |
| Healthy control | | (n = 20/group) | 54 ± 4.2 | 54 ± 4.2 |
| Healthy control | | (n = 20/group) | 63 ± 9.9 | 64 ± 9.9 |
| Healthy control | | (n = 1/group) | 67 | 67 |
| Healthy control | | (n = 1/group) | 61 | 61 |
| Healthy control | | (n = 1/group) | 69 | 69 |

TABLE 7
IPA interaction networks of plasma Ig-bound
citrullinome in breast cancer patients.

| | | Focus | |
|---|---|---|---|
| ID | Score | Molecules | Top Diseases and Functions |
| Luminal A | | | |
| 1 | 60 | 25 | [Cell Morphology, Embryonic |
| | | | Development, Hair and Skin |
| | | | Development and Function] |
| 2 | 28 | 14 | [Drug Metabolism, Endocrine System |
| | | | Development and Function, |
| | | | Energy Production] |
| TNBC | | | |
| 1 | 44 | 17 | [Cell Morphology, Embryonic |
| | | | Development, Hair and Skin |
| | | | Development and Function] |
| 2 | 26 | 11 | [Cancer, Gene Expression, Organismal |
| | | | Injury and Abnormalities] |
| HR−/Her2 | | | |
| 1 | 72 | 30 | [Cell Morphology, Embryonic |
| | | | Development, Hair and Skin |
| | | | Development and Function] |
| 2 | 47 | 22 | [Cellular Compromise, Cellular |
| | | | Function and Maintenance, |
| | | | Inflammatory Response] |

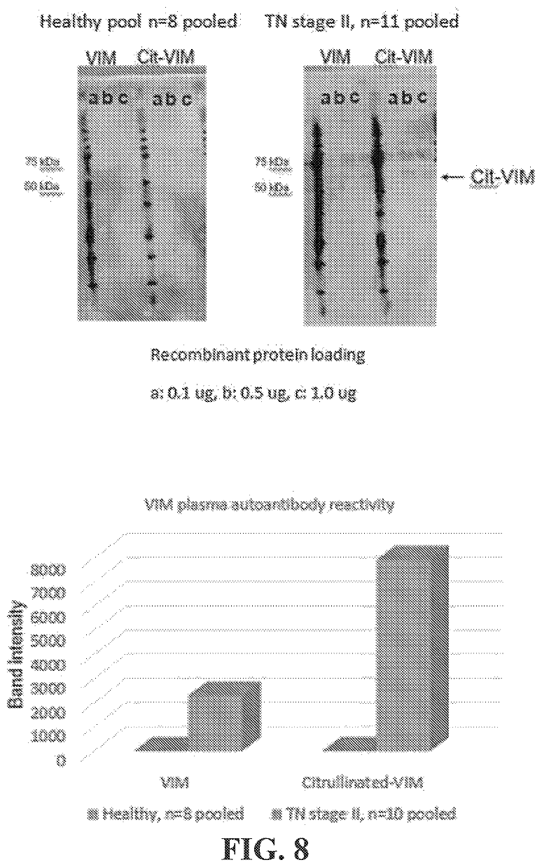

To determine the extent to which autoantibody reactivity is directed against citrulline-containing epitopes, we first used a western blot approach and evaluated differential autoantibody reactivity against citrullinated- and non-citrullinated vimentin using plasma pools from patients with newly diagnosed stage II TNBC (n=11 subjects/pool) (Table 8) and healthy controls (n=8 subjects/pool). We chose to focus on vimentin as our antigen of interest as autoantibody reactivity against vimentin has previously been reported in autoimmune disease and cancer (refs. 43-46). The high autoantibody reactivity against citrullinated vimentin was observed in the TNBC stage II patient plasma pool compared with the healthy control pool (FIG. 8). We note that immunoblots using plasma primary autoantibodies can cause considerable background due to reactivity of autoantibodies that are common between healthy control and patients with cancer. Therefore, we additionally developed Luminex autoantibody ELISA assays to test autoantibody reactivity against citrullinated- and non-citrullinated vimentin using individual plasmas from patients with newly diagnosed stage II TNBC (n=11) (Table 8) and healthy controls (n=31). Autoantibody reactivity against citrullinated vimentin was statistically significantly elevated in cases compared with controls (Wilcoxon rank-sum test, two-sided p=0.01 with an AUC of 0.75 (95% CI: 0.58 to 0.92); FIGS. 4C and 4D.); and compared with non-citrullinated vimentin among cases (2-sided paired t-test<0.001) (FIG. 4E).

TABLE 8
TNBC stage II cohort used for the plasma citrullinated vimentin ELISA assay.

| | | | | | | | T Size | |
|---|---|---|---|---|---|---|---|---|
| Menopausal | | | Clin | Clin | Clin | Clin | Baseline_{—} | Age |
| status | Trigger event | Histology | T | N | M | STAGE | Neoadj. | at DX |
| PRE | ABNORMAL | INVASIVE | T2 | N0 | M0 | IIA | 3.4 | 46 |
| | MAMMOGRAM | DUCTAL, | | | | | | |
| | | NOS | | | | | | |
| PRE | LUMP IN | INVASIVE | T3 | N0 | M0 | IIB | 6 | 45 |
| | BREAST (PT) | DUCTAL, | | | | | | |
| | | NOS | | | | | | |
| PRE | LUMP IN | INVASIVE | T2 | N1 | M0 | IIB | 3.8 | 50 |
| | BREAST (PT) | DUCTAL, | | | | | | |
| | | NOS | | | | | | |
| PRE | ABNORMAL | INVASIVE | T2 | N0 | M0 | IIA | 3.3 | 44 |
| | MAMMOGRAM | DUCTAL, | | | | | | |
| | | NOS | | | | | | |
| POST | LUMP IN | INVASIVE | T2 | N0 | M0 | IIA | 4.4 | 64 |
| | BREAST (PT) | DUCTAL, | | | | | | |
| | | NOS | | | | | | |
| POST | LUMP IN | INVASIVE | T2 | N1 | M0 | IIB | 4.5 | 62 |
| | BREAST (PT) | DUCTAL, | | | | | | |
| | | NOS | | | | | | |
| POST | LUMP IN | INVASIVE | T2 | N0 | MO | IIA | 2.1 | 65 |
| | BREAST (PT) | DUCTAL, | | | | | | |
| | | NOS | | | | | | |
| POST | LUMP IN | INVASIVE | T2 | N0 | M0 | IIA | 4 | 64 |
| | BREAST (PT) | DUCTAL, | | | | | | |
| | | NOS | | | | | | |
| POST | ABNORMAL | INVASIVE | Tlc | N1 | M0 | IIA | | 54 |
| | MAMMOGRAM | DUCTAL, | | | | | | |
| | | NOS | | | | | | |
| POST | ABNORMAL | INVASIVE | Tlc | N1 | M0 | IIA | | 54 |
| | MAMMOGRAM | DUCTAL | | | | | | |
| | | NOS | | | | | | |
| POST | ABNORMAL | INVASIVE | T2 | N0 | M0 | IIA | 2.1 | 68 |
| | MAMMOGRAM | DUCTAL, | | | | | | |
| | | NOS | | | | | | |

### Example 7. Citrullinated Peptides May be Useful for Cancer Vaccine Development

We have experimentally confirmed the occurrence of citrullinated mass spectrometry identified peptide fragments of ENO1 in the cell surfaceome, immunopeptidome of breast cancer cell lines, and circulating plasma IgG bound citrullinated peptides among TNBC cases compared to healthy controls (refs 52, 53). All identified peptides in the immunopeptidome were found to be greater than 12 amino acids in length and thus citrullinated peptides were exclusively class II peptides and suitable for peptide vaccination (refs 54, 55).

In some cases, the experimentally identified peptides described above may not be the best amino acid length for vaccination since protease digestion was used to make the proteins and peptides the proper length for detection via mass spectrometry. To synthesize the peptides for ELISpot assays in best length of 22 to 24 amino acid length used in many of vaccination studies, we additionally performed in silico binding affinity predictions between peptides and MHC-II molecules using the well-established prediction tool NetMHC-IIpanV.2.3. software(refs 56, 57). Briefly, identified citrullinated peptide sequences were considered as unmodified form and loaded to the prediction tool, followed by sorting the binding peptide core affinity prediction (IC50, nM) with MHC-II pocket assessed by the artificial intelligent network SSNAlignment, and the percentile rank that was generated by comparing the peptide's score against the scores of one million random 15 mers selected from Swiss-Prot database.

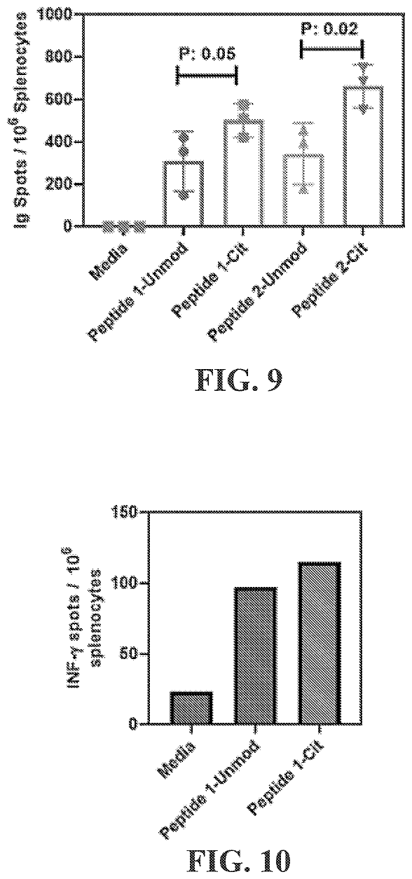

Using ELISpot assays, we tested the unmodified and citrullinated forms of three pairs of synthesized ENO1 peptides predicted as having high MHC-II binding affinity and assessed B cell-mediated IgG and T cell-mediated IFNγ secretion (Table 9). Two of the three citrullinated peptides (peptide-1 and -2) elicited a potent IgG response; none of the unmodified peptides showed appreciable differences compared to media (FIG. 9). A modest increase in IFNg secretion was observed with citrullinated peptide-1 (FIG. 10).

TABLE 9
Unmodified and citrullinated peptides tested in ELISpot assays.

| | | | Synthesized | | | |
|---|---|---|---|---|---|---|
| | | | Peptide | | Modi- | Mass Spectrometry identified |
| Peptide | Gene | Position | Sequence | Length | fication | ENO1 cit-peptide sequence |
| Peptide | ENO1_ | 32-56 | RAAVPSGASTGI | 25 | None | |
| 1 | WT | | YEALELRDNDK | | | |
| | | | TR (SEQ ID | | | |
| | | | NO: 136) | | | |
| Peptide | ENO1_ | 32-56 | R*AAVPSGASTG | 25 | R1(Cit.), | FTSKGLFR*AAVPSGASTGIYE |
| 1 | CIT | | IYEALELR*DND | | R19(Cit.), | (SEQ ID NO: 42) |
| | | | KTR* (SEQ ID | | R56(Cit.) | |
| | | | NO: 137) | | | |
| Peptide | ENO1_ | 164-86 | AMQEFMILPVG | 22 | None | |
| 2 | WT | | ASSFREAMRGA | | | |
| | | | (SEQ ID NO: 138) | | | |
| Peptide | ENO1_ | 164-86 | AMQEFMILPVG | 22 | R16(Cit.), | HIADLAGNSEVILPVPAFNVINGGSHA |
| 2 | CIT | | ASSFR*EAMR*G | | R20(Cit.) | GNKLAMQEFMILPVGAANFR*EAMR |
| | | | A (SEQ ID | | | (SEQ ID NO: 142) |
| | | | NO: 139) | | | |
| Peptide | ENO1_ | 262-284 | (SEQ ID NO: 140) | 23 | None | |
| 3 | WT | | KSPDDPSRYITP | | | |
| | | | DQLADLYKSFV | | | |
| Peptide | ENO1_ | 262-284 | KSPDDPSR*YITP | 23 | R8(Cit.) | SPDDPSR*YISPDQLADLYK (SEQ ID |
| 3 | CIT | | DQLADLYKSFV | | | NO: 143) |
| | | | (SEQ ID NO: 141) | | | |

Cit.: citrullinated Arg. (represented as R*)

### Example 8. Identifying Additional Cancer Cell Specific Citrullinated Protein Immunotherapy Targets

Cancer immunotherapy approaches which target overexpressed proteins on the surface of cancer cells have shown enormous clinical efficacy. However, expression of these proteins is generally not limited to cancer cells (e.g., tumors) and may also occur at a low to moderate level in normal tissues. In these cases, targeting of these proteins in immunotherapy approaches often causes negative side effects. As described above, PADI family enzymes are highly overexpressed in various cancer types and not expressed in normal tissues, which means that citrullinated proteins expressed on the surface of cancer cells may be preferable as immunotherapy targets.

Table 10 shows citrullinated sites on representative proteins that are commonly targeted in cancer immunotherapy, including TACSTD2 (TROP2), EGFR, ERBB2 and various other targets. Cancer cell surface proteins were biotin labeled, purified using streptavidin, digested with trypsin, and analyzed via mass spectrometry. The mass spectra were searched against the Uniprot Human genome database, with arginine citrullination considered as a variable modification. Multiple citrullination sites were identified from those proteins in various cancer types, and the citrullination modifications on those proteins were unreported elsewhere. Targeting citrullinated forms of proteins expressed on the surface of cancer cells (e.g., tumors) is a unique approach which further increases the specificity of cancer cell targeting (i.e., compared to targeting of normal cells, which can cause side effects).

TABLE 10
Citrullinated peptides identified in cancer immunotherapy target proteins.

| Gene | Sequence | Modification |
|---|---|---|
| TACSTD2 | TLVR*PSEHALVDNDGLYDPDCDPEGR* (SEQ ID NO: 144) | R4(Cit.); |
| (TROP2) | | R26(Cit.) |
| TACSTD2 | HR*PTAGAFNHSDLDAELR* (SEQ ID NO: 145) | R2(Cit.); |
| (TROP2) | | R18(Cit.) |
| TACSTD2 | FVAAVHYEQPTIQIELR* (SEQ ID NO: 146) | R17(Cit.) |
| (TROP2) | | |
| TACSTD2 | AAGDVDIGDAAYYFER* (SEQ ID NO: 147) | R16(Cit.) |
| (TROP2) | | |
| TACSTD2 | DIKGESLFQGR*GGLDLR* (SEQ ID NO: 148) | R11(Cit.); |
| (TROP2) | | R17(Cit.) |
| TACSTD2 | R*LTAGLIAVIVVVVVALVAGMAVLVITNR (SEQ ID NO: 149) | R1(Cit.) |
| TROP2) | | |
| EGFR | LTQLGTFEDHELSLQR* (SEQ ID NO: 150) | R16(Cit.) |
| EGFR | TIQEVAGYVLIALNTVER* (SEQ ID NO: 151) | R18(Cit.) |
| EGFR | IPLENLQIIR*GNMYYENSYALAVLSNYDANKTGLK (SEQ ID NO: 152) | R10(Cit.) |
| EGFR | FSNNPALCNVESIQWR* (SEQ ID NO: 153) | R16(Cit.) |
| EGFR | SPSDCCHNQCAAGCTGPR*ESDCLVCR* (SEQ ID NO: 154) | R18(Cit.); |
| | | R26(Cit.) |
| EGFR | YSFGATCVKKCPR*NYVVTDHGSCVR* (SEQ ID NO: 155) | R13(Cit.); |
| | | R25(Cit.) |
| EGFR | NYVVTDHGSCVRACGADSYEMEEDGVR* (SEQ ID NO: 156) | R27(Cit.) |
| EGFR | ATGQVCHALCSPEGCWGPEPR*DCVSCR*NVSR* (SEQ ID NO: 157) | R21(Cit.); |
| | | R27(Cit.); |
| | | R31(Cit.) |
| ERBB2 | CWGESSEDCQSLTR* (SEQ ID NO: 158) | R14(Cit.) |
| ERBB2 | YTFGASCVTACPYNYLSTDVGSCTL VCPLHNQEVTAEDGTQR* (SEQ ID | R42(Cit.) |
| | NO: 159) | |
| ERBB2 | CEKCSKPCAR*VCYGLGMEHLR (SEQ ID NO: 160) | R10(Cit.) |
| ERBB2 | VCYGLGMEHLR*EVR*AVTSANIQEFAGCK (SEQ ID NO:1 61) | R11(Cit.); |
| | | R14(Cit.) |
| ERBB2 | ILHNGAYSLTLOGLGISWLGLR* (SEQ ID NO: 162) | R22(Cit.) |
| ERBB2 | ELGSGLALIHHNTHLCFVHTVPWDQLFR* (SEQ ID NO: 163) | R28(Cit.) |
| ERBB2 | GHCWGPGPTQCVNCSQFLR* (SEQ ID NO: 164) | R19(Cit.) |
| NECTIN4 | GDSGEQVGQVAWAR* (SEQ ID NO: 165) | R14(Cit.) |
| NECTIN4 | NAVQADEGEYECR* (SEQ ID NO: 166) | R13(Cit.) |
| NECTIN4 | SMNGQPLTCVVSHPGLLQDQR* (SEQ ID NO: 167) | R21(Cit.) |
| NECTIN4 | ITHILHVSFLABASVR* (SEQ ID NO: 168) | R16(Cit.) |
| NECTIN4 | GLEDQNLWHIGR* (SEQ ID NO: 169) | R12(Cit.) |
| NECTIN4 | VDGDTLGFPPLTTEHSGIYVCHVSNEFSSR* (SEQ ID NO: 170) | R30(Cit.) |
| CD44 | FAGVFHVEKNGRYSISR* (SEQ ID NO: 171) | R17(Cit.) |
| (CSPG8) | | |
| CD44 | DGTR*YVOKGEYR* (SEQ ID NO: 172) | R4(Cit.); |
| (CSPG8) | | R12(Cit.) |
| CD44 | TNPEDIYPSNPTDDDVSSGSSSER* (SEQ ID NO: 173) | R24(Cit.) |
| (CSPG8) | | |
| CEACAM5 | VDGNR*QIIGYVIGTQQATPGPAYSGR* (SEQ ID NO: 174) | R5(Cit.); |
| | | R26(Cit.) |
| CEACAM5 | SDLVNEEATGQFR* (SEQ ID NO: 175) | R13(Cit.) |
| CEACAM5 | NDTASYKCETQNPVSAR* (SEQ ID NO: 176) | R17(Cit.) |
| CEACAM5 | R*SDSVILNVLYGPDAPTISPLNTSYR* (SEQ ID NO: 177) | R1(Cit.); |
| | | R26(Cit.) |
| HSP90AA1 | QDR*TLTIVDTGIGMTK (SEQ ID NO: 178) | R3(Cit.) |
| (HSP90) | | |
| HSP90AA1 | VTVITKHNDDEQYAWESSAGGSFTVR*TDTGEPMGR*GTK (SEQ ID | R26(Cit.); |
| (HSP90) | NO: 179) | R35(Cit.) |
| HSP90AA1 | HSQFIGYPITLFVEKER*DKEVSDDEAEEK (SEQ ID NO: 180) | R17(Cit.) |
| (HSP90) | | |
| HSP90AA1 | SLINDWEDHLAVKHFSVEGQLEFR* (SEQ ID NO: 181) | R24(Cit.) |
| (HSP90) | | |
| HSP90AA1 | R*VFIMDNCEELIPEYLNFIR* (SEQ ID NO: 182) | R1(Cit.); |
| (HSP90) | | R20(Cit.) |
| HSP90AA1 | GVVDSEDLPLNISR* (SEQ ID NO: 183) | R14(Cit.) |
| (HSP90) | | |
| HSP90AA1 | KLSELLR*YYTSASGDEMVSLKDYCTR* (SEQ ID NO: 184) | R7(Cit.); |
| (HSP90) | | R26(Cit.) |
| HSP90AA1 | VVVSNR*LVTSPCCIVTSTYGWTANMER*IMKAQALR | R6(Cit.); |
| (HSP90) | (SEQ ID NO: 185) | R27(Cit.) |
| HSP90AA1 | AQALR*DNSTMGYMAAK (SEQ ID NO: 186) | R5(Cit.) |
| (HSP90) | | |
| HSP90AA1 | SR*APVNWYQEK (SEQ ID NO: 187) | R2(Cit.) |
| (HSP90) | | |
| Isoform 2 | | |
| CD99 | KPSAGDDFDLGDAVVDGENDDPR*PPNPPKPMPNPNPNHPSSSGSFSDAD | R23(Cit.) |
| | LADGVSGGEGK (SEQ ID NO: 188) | |
| CD99 | ENAEQGEVDMESHR*NANAEPAVQR* (SEQ ID NO: 189) | R14(Cit.); |
| | | R24(Cit.) |
| IGF1R | VAGLESLGDLFPNLTVIR*GWK (SEQ ID NO: 190) | R18(Cit.) |
| IGF1R | MEEVTGTKGR*QSKGDINTR* (SEQ ID NO: 191) | R10(Cit.); |
| | | R19(Cit.) |
| IGF1R | GROSKGDINTR*NNGER* (SEQ ID NO: 192) | R11(Cit.); |
| | | R16(Cit.) |
| IGF1R | YGSQVEDQR*ECVSRQEYR* (SEQ ID NO: 193) | R9(Cit.); |
| | | R18(Cit.) |
| CD22 | SPLSTLTVYYSPETIGR* (SEQ ID NO: 194) | R17(Cit.) |
| CD22 | YCCQVSNDVGPGR* (SEQ ID NO: 195) | R13(Cit.) |
| CD33 | LDQEVQEETQGRFR*LLGDPSR* (SEQ ID NO: 196) | R14(Cit.); |
| | | R21(Cit.) |

### REFERENCES CITED IN THIS DISCLOSURE

1. Malmstrom V, Catrina Al, Klareskog L. The immunopathogenesis of seropositive rheumatoid arthritis: from triggering to targeting. Nature reviews Immunology 2017; 17(1):60-75. doi: 10.1038/nri.2016.124 [published Online First: 2016 Dec. 6]
2. Yuzhalin A E. Citrullination in Cancer. Cancer Res 2019; 79(7):1274-84. doi: 10.1158/0008-5472.CAN-18-2797 [published Online First: 2019 Mar. 22]
3. Schellekens G A, de Jong B A, van den Hoogen F H, et al. Citrulline is an essential constituent of antigenic determinants recognized by rheumatoid arthritis-specific autoantibodies. The Journal of clinical investigation 1998; 101(1):273-81. doi: 10.1172/jci1316 [published Online First: 1998 Feb. 14]
4. Witalison E E, Thompson P R, Hofseth L J. Protein Arginine Deiminases and Associated Citrullination: Physiological Functions and Diseases Associated with Dysregulation. Current drug targets 2015; 16(7):700-10. doi: 10.2174/1389450116666150202160954 [published Online First: 2015 Feb. 3]
5. Scherer H U, Huizinga T W J, Krönke G, et al. The B cell response to citrullinated antigens in the development of rheumatoid arthritis. Nature Reviews Rheumatology 2018; 14:157. doi: 10.1038/nrrheum.2018.10
6. Margulies D H. How MHC molecules grab citrullinated peptides to foster rheumatoid arthritis. The Journal of biological chemistry 2018; 293(9):3252-53. doi: 10.1074/jbc.H118.001829 [published Online First: 2018 Mar. 4]
7. Alspach E, Lussier D M, Miceli A P, et al. MHC-H neoantigens shape tumour immunity and response to immunotherapy. Nature 2019 doi: 10.1038/s41586-019-1671-8 [published Online First: 2019 Oct. 28]
8. Li P, Wang D, Yao H, et al. Coordination of PAD4 and HDAC2 in the regulation of p53-target gene expression. Oncogene 2010; 29(21):3153-62. doi: 10.1038/onc.2010.51 [published Online First: 2010 Mar. 2]
9. Wang Y, Wysocka J, Sayegh J, et al. Human PAD4 regulates histone arginine methylation levels via demethylimination. Science (New York, NY) 2004; 306(5694):279-83. doi: 10.1126/science.1101400 [published Online First: 2004 Sep. 4]
10. Guo Q, Fast W. Citrullination of inhibitor of growth 4 (ING4) by peptidylarginine deminase 4 (PAD4) disrupts the interaction between ING4 and p53. The Journal of biological chemistry 2011; 286(19):17069-78. doi: 10.1074/jbc.M111.230961 [published Online First: 2011 Apr. 2]
11. Tanikawa C, Espinosa M, Suzuki A, et al. Regulation of histone modification and chromatin structure by the p53-PADI4 pathway. Nature communications 2012; 3:676. doi: 10.1038/ncomms1676 [published Online First: 2012 Feb. 16]
12. Brentville V A, Metheringham R L, Gunn B, et al. Citrullinated Vimentin Presented on MHC-II in Tumor Cells Is a Target for CD4+ T-Cell-Mediated Antitumor Immunity. Cancer Res 2016; 76(3):548-60. doi: 10. 1158/0008-5472.CAN-15-1085 [published Online First: 2016 Jan. 1]
13. Cook K, Daniels I, Symonds P, et al. Citrullinated alpha-enolase is an effective target for anti-cancer immunity. Oncoimmunology 2018; 7(2):e1390642. doi: 10.1080/2162402X.2017.1390642 [published Online First: 2018 Jan. 9]
14. Tsou P, Katayama H, Ostrin E J, et al. The Emerging Role of B Cells in Tumor Immunity. Cancer Res 2016; 76(19):5597-601. doi: 10.1158/0008-5472.CAN-16-0431 [published Online First: 2016 Sep. 17]
15. Disis M L, Stanton S E. Immunotherapy in breast cancer: An introduction. Breast 2018; 37:196-99. doi: 10.1016/j.breast.2017.01.013 [published Online First: 2017 Feb. 7]
16. Ryan S O, Turner M S, Gariepy J, et al. Tumor antigen epitopes interpreted by the immune system as self or abnormal-self differentially affect cancer vaccine responses. Cancer Res 2010; 70(14):5788-96. doi: 10.1158/0008-5472.CAN-09-4519 [published Online First: 2010 Jul. 1]
17. Binnewies M, Roberts E W, Kersten K, et al. Understanding the tumor immune microenvironment (TIME) for effective therapy. Nat Med 2018; 24(5):541-50. doi: 10.1038/s41591-018-0014-x [published Online First: 2018 Apr. 25]
18. Derks S, de Klerk L K, Xu X, et al. Characterizing diversity in the tumor-immune microenvironment of distinct subclasses of gastroesophageal adenocarcinomas. Ann Oncol 2020; 31(8):1011-20. doi: 10.1016/j.annonc.2020.04.011 [published Online First: 2020 May 11]
19. Faca V M, Hanash S M. In-depth proteomics to define the cell surface and secretome of ovarian cancer cells and processes of protein shedding. Cancer Res 2009; 69(3):728-30. doi: 10.1158/0008-5472.CAN-08-3087 [published Online First: 2009 Jan. 22]
20. Wang H, Hanash S. Mass spectrometry based proteomics for absolute quantification of proteins from tumor cells. Methods 2015; 81:34-40. doi: 10.1016/j.ymeth.2015.03.007 [published Online First: 2015 Mar. 22]
21. Pitteri S J, JeBailey L, Faca V M, et al. Integrated proteomic analysis of human cancer cells and plasma from tumor bearing mice for ovarian cancer biomarker discovery. PLoS One 2009; 4(11):e7916. doi: 10.1371/journal.pone.0007916 [published Online First: 2009 Nov. 26]
22. Taguchi A, Delgado O, Celiktas M, et al. Proteomic signatures associated with p53 mutational status in lung adenocarcinoma. Proteomics 2014; 14(23-24):2750-9. doi: 10.1002/pmic.201400378 [published Online First: 2014 Oct. 22]
23. Katayama H, Tsou P, Kobayashi M, et al. A plasma protein derived TGFbeta signature is a prognostic indicator in triple negative breast cancer. NPJ Precis Oncol 2019; 3:10. doi: 10.1038/s41698-019-0082-5 [published Online First: 2019 Apr. 10]
24. Katayama H, Boldt C, Ladd J J, et al. An Autoimmune Response Signature Associated with the Development of Triple-Negative Breast Cancer Reflects Disease Pathogenesis. Cancer Res 2015; 75(16):3246-54. doi: 10.1158/0008-5472.CAN-15-0248 [published Online First: 2015 Jun. 20]
25. Ladd J J, Chao T, Johnson M M, et al. Autoantibody signatures involving glycolysis and splicesome proteins precede a diagnosis of breast cancer among postmenopausal women. Cancer Res 2013; 73(5):1502-13. doi: 10.1158/0008-5472.Can-12-2560 [published Online First: 2012 De. 28]
26. Tripathi S C, Peters H L, Taguchi A, et al. Immunoproteasome deficiency is a feature of non-small cell lung cancer with a mesenchymal phenotype and is associated with a poor outcome. Proc Natl Acad Sci USA 2016; 113(11):E1555-64. doi: 10.1073/pnas.1521812113 [published Online First: 2016 Mar. 2]
27. Nielsen M, Lundegaard C, Lund O. Prediction of MHC class 11 binding affinity using SMM-align, a novel stabilization matrix alignment method. BMC Bioinformatics 2007:8:238. doi: 10.1186/1471-2105-8-238 [published Online First: 2007 Jul. 5]
28. Jensen K K, Andreatta M, Marcatili P, et al. Improved methods for predicting peptide binding affinity to MHC class II molecules. Immunology 2018; 154(3):394-406. doi: 10.1111/imm.12889 [published Online First: 2018 Jan. 10]
29. Katono K, Sato Y, Jiang S X, et al. Prognostic significance of MYH9 expression in resected non-small cell lung cancer. PLoS One 2015; 10(3):e0121460. doi: 10.1371/journal.pone.0121460 [published Online First: 2015 Apr. 1]
30. Gao J, Aksoy B A, Dogrusoz U, et al. Integrative analysis of complex cancer genomics and clinical profiles using the cBioPortal. Science signaling 2013; 6(269):pl1. doi: 10.1126/scisignal.2004088 [published Online First: 2013 Apr. 4]
31. Curtis C, Shah S P, Chin S F, et al. The genomic and transcriptomic architecture of 2,000 breast tumours reveals novel subgroups. Nature 2012; 486(7403):346-52. doi: 10.1038/nature10983 [published Online First: 2012 Apr. 24]
32. Rhodes D R, Yu J, Shanker K, et al. ONCOMINE: a cancer microarray database and integrated data-mining platform. Neoplasia (New York, NY) 2004; 6(1):1-6. [published Online First: 2004 Apr. 8]
33. Fahrmann J F, Vykoukal J, Fleury A, et al. Association between plasma diacetylspermine and tumor spermine synthase with outcome in triple negative breast cancer. J Natl Cancer Inst 2019 doi: 10.1093/jnci/djz182 [published Online First: 2019 Sep. 11]
34. Bindea G, Mlecnik B, Tosolini M, et al. Spatiotemporal dynamics of intratumoral immune cells reveal the immune landscape in human cancer. Immunity 2013; 39(4):782-95. doi: 10.1016/j.immuni.2013.10.003 [published Online First: 2013 Oct. 22]
35. Chen F, Zhang Y, Bosse D, et al. Pan-urologic cancer genomic subtypes that transcend tissue of origin. Nature communications 2017; 8(1):199. doi: 10.1038/s41467-017-00289-x [published Online First: 2017 Aug. 5]
36. Curtis C, Shah S P, Chin S F, et al. The genomic and transcriptomic architecture of 2,000 breast tumours reveals novel subgroups. Nature 2012; 486(7403):346-52. doi: 10.1038/nature10983 [published Online First: 2012 Apr. 24]
37. Kramer A, Green J, Pollard J, Jr., et al. Causal analysis approaches in Ingenuity Pathway Analysis. Bioinformatics (Oxford, England) 2014; 30(4):523-30. doi: 10.1093/bioinformatics/btt703 [published Online First: 2013 Dec. 18]
38. Feitsma A L, van der Voort E I, Franken K L, et al. Identification of citrullinated vimentin peptides as T cell epitopes in HLA-DR4-positive patients with rheumatoid arthritis. Arthritis and rheumatism 2010; 62(1):117-25. doi: 10.1002/art.25059 [published Online First: 2009 Dec. 30]
39. Lundberg K, Kinloch A, Fisher B A, et al. Antibodies to citrullinated alpha-enolase peptide 1 are specific for rheumatoid arthritis and cross-react with bacterial enolase. Arthritis and rheumatism 2008:58(10):3009-19. doi: 10.1002/art.23936 [published Online First: 2008 Sep. 30]
40. Kinloch A, Tatzer V, Wait R, et al. Identification of citrullinated alpha-enolase as a candidate autoantigen in rheumatoid arthritis. Arthritis research & therapy 2005; 7(6):R1421-9. doi: 10.1186/ar1845 [published Online First: 2005 Nov. 10]
41. Capello M, Vykoukal J V, Katayama H, et al. Exosomes harbor B cell targets in pancreatic adenocarcinoma and exert decoy function against complement-mediated cytotoxicity. Nature communications 2019; 10(1):254. doi: 10.1038/s41467-018-08109-6 [published Online First: 2019 Jan. 18]
42. Kwa M J, Adams S. Checkpoint inhibitors in triple-negative breast cancer (TNBC): Where to go from here. Cancer 2018; 124(10):2086-103. doi: 10.1002/cncr.31272 [published Online First: 2018 Feb. 10]
43. Hong S H, Misek D E, Wang H, et al. Identification of a Specific Vimentin Isoform That Induces an Antibody Response in Pancreatic Cancer. Biomark Insights 2006; 1:175-83. [published Online First: 2008 Sep. 5]
44. Bretherton L, Toh B H, Jack 1. IgM autoantibody to intermediate filaments in Mycoplasma pneumoniae infections. Clin Immunol Immunopathol 1981; 18(3):425-30. doi: 10.1016/0090-1229(81)90135-5 [published Online First: 1981 Mar. 1]
45. Alcover A, Molano J, Renart J, et al. Antibodies to vimentin intermediate filaments in sera from patients with systemic lupus erythematosus. Arthritis Rheum 1984:27(8):922-8. doi: 10.1002/art.1780270812 [published Online First: 1984 Aug. 1]
46. Kurki P, Helve T, Virtanen I. Antibodies to cytoplasmic intermediate filaments in rheumatic diseases. J Rheumatol 1983; 10(4):558-62. [published Online First: 1983 Aug. 1]
47. Stadler S C, Vincent C T, Fedorov V D, et al. Dysregulation of PAD4-mediated citrullination of nuclear GSK3beta activates TGF-beta signaling and induces epithelial-to-mesenchymal transition in breast cancer cells. Proc Natl Acad Sci USA 2013; 110(29):11851-6. doi: 10.1073/pnas.1308362110 [published Online First: 2013 Jul. 3]
48. van Venrooij W J, van Beers J J, Pruijn G J. Anti-CCP antibodies: the past, the present and the future. Nature reviews Rheumatology 2011; 7(7):391-8. doi: 10.1038/nrrheum.2011.76 [published Online First: 2011 Jun. 8]
49. Monteith G R, McAndrew D, Faddy H M, et al. Calcium and cancer: targeting Ca2+transport. Nat Rev Cancer 2007; 7(7):519-30. doi: 10.1038/nrc2171 [published Online First: 2007 Jun. 23]
50. Friou G J, Finch S C, Detre K D. Interaction of nuclei and globulin from lupus erythematosis serum demonstrated with fluorescent antibody. Journal of immunology (Baltimore, Md: 1950) 1958; 80(4):324-9. [published Online First: 1958 Apr. 1]
51. Vlagea A, Falagan S, Gutierrez-Gutierrez G, et al. Antinuclear antibodies and cancer: A literature review. Critical reviews in oncology/hematology 2018; 127:42-49. doi: 10.1016/j.critrevonc.2018.05.002 [published Online First: 2018 Jun. 13]
52. Katayama H, Kobayashi M, Irajizad E, Sevillarno A, Patel N, Mao X, et al. Protein citrullination as a source of cancer neoantigens. J Immunother Cancer 2021; 9(6) doi 10.1136/jitc-2021-002549.
53. Katayama H, Tsou P, Kobayashi M, Capello M, Wang H, Esteva F, et al. A plasma protein derived TGFβ signature is a prognostic indicator in triple negative breast cancer. NPJ Precis Oncol 2019; 3:10 doi 10.1038/s41698-019-0082-5.
54. Gil E Y, Jo U H, Lee H J, Kang J, Seo J H, Lee E S, et al. Vaccination with ErbB-2 peptides prevents cancer stem cell expansion and suppresses the development of spontaneous tumors in MMTV-PyMT transgenic mice. Breast Cancer Res Treat 2014; 147(1):69-80 doi 10.1007/s10549-014-3086-4.
55. Ebben J D, Lubet R A, Gad E, Disis M L, You M. Epidermal growth factor receptor derived peptide vaccination to prevent lung adenocarcinoma formation: An in vivo study in a murine model of EGFR mutant lung cancer. Mol Carcinog 2016; 55(11):1517-25 doi 10.1002/mc.22405.
56. Nielsen M, Lundegaard C, Lund O. Prediction of MHC class 11 binding affinity using SMM-align, a novel stabilization matrix alignment method. BMC Bioinformatics 2007:8:238 doi 10.1186/1471-2105-8-238.
57. Jensen K K, Andreatta M, Marcatili P, Buus S, Greenbaum J A, Yan Z, et al. Improved methods for predicting peptide binding affinity to MHC class II molecules. Immunology 2018; 154(3):394-406 doi 10.1111/imm.12889.

The following U.S. patents and published patent applications are hereby incorporated herein by reference: U.S. Pat. Nos. 6,872,385; 7,547,759; 9,562,070; 9,629,877; 10,239,916; 11,155,599; US 2019/0093085, U.S. Pat. Nos. 10,088,479 and 10,695,438.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

Disclosed herein are materials, compositions, and methods that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are various additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

1. A method, comprising:
providing a plasma sample from a patient suffering from a cancer or suspected of suffering from the cancer;
incubating the plasma sample with at least one citrullinated protein selected from the group consisting of the proteins encoded by genes listed in Table 1, the proteins encoded by genes listed in Table 2, and the proteins encoded by genes listed in Table 10, under conditions sufficient for any autoantibodies against the citrullinated protein(s) that may be present in the plasma sample, to bind to the citrullinated protein(s);
incubating the citrullinated protein(s) and any bound autoantibodies against it/them with a detectable label, under conditions in which the detectable label will bind to the bound autoantibodies and will substantially not bind to other molecules;
detecting the detectable label bound to the bound autoantibodies;
classifying the patient as suffering from the cancer in response to the detected amount of the detectable label bound to the bound autoantibodies being equal to or greater than a threshold; and
classifying the patient as not suffering from the cancer in response to the detected amount of the detectable label bound to the bound autoantibodies being less than the threshold.

2. The method of claim 1, wherein:
the cancer is selected from the group consisting of breast cancers, lung cancers, skin cancers, endometrial cancers, ovarian cancers, and colorectal cancers.

3. The method of claim 2, wherein:
the citrullinated protein(s) is/are selected from the group consisting of citrullinated vimentin and citrullinated α-enolase.

4. The method of claim 1, wherein the detectable label is an antibody against the autoantibody, wherein the antibody comprises a fluorescent moiety.

5. The method of claim 1, further comprising administering, to the patient, a cancer treatment selected from the group consisting of surgical resection, chemotherapy, monoclonal antibody therapy, checkpoint inhibitor therapy, oncolytic virus therapy, thermal therapy, radiotherapy, a cancer vaccine comprising the citrullinated protein(s), a targeted therapy against the citrullinated protein(s),and two or more thereof, in response to classifying the patient as suffering from the cancer; and
testing the patient for at least one other medical condition, in response to classifying the patient as not suffering from the cancer.

6. A kit, comprising:
a substrate;
at least one citrullinated protein selected from the group consisting of the proteins encoded by genes listed in Table 1, the proteins encoded by genes listed in Table 2, and the proteins encoded by genes listed in Table 10; and
instructions for performing a method, comprising instructions to:
provide a plasma sample from a patient suffering from a cancer or suspected of suffering from the cancer;
incubate the plasma sample with the citrullinated protein(s) under conditions sufficient for any autoantibodies against the citrullinated protein(s) that may be present in the plasma sample, to bind to the citrullinated protein(s);
incubate the citrullinated protein(s) and any bound autoantibodies against it/them with a detectable label, under conditions wherein the detectable label will bind to the bound autoantibodies and will substantially not bind to other molecules;
detect the label bound to the bound autoantibodies;
classify the patient as suffering from the cancer, in response to the detected amount of the label bound to the bound autoantibodies being equal to or greater than a threshold; and
classify the patient as not suffering from the cancer, in response to the detected amount of the label bound to the bound autoantibodies being less than the threshold.

7. The kit of claim 6, wherein:
the cancer is selected from the group consisting of breast cancers, lung cancers, skin cancers, endometrial cancers, ovarian cancers, and colorectal cancers.

8. The kit of claim 7, wherein:
the citrullinated protein is selected from the group consisting of citrullinated vimentin and citrullinated α-enolase.

9. The kit of claim 6, wherein the detectable label is an antibody against the autoantibody, wherein the antibody comprises a fluorescent moiety.

10. A method, comprising:
providing a tissue sample from a patient suffering from a cancer or suspected of suffering from the cancer;
assaying the tissue sample for at least one citrullinated protein selected from the group consisting of the proteins encoded by genes listed in Table 1, the proteins encoded by genes listed in Table 2, and the proteins encoded by genes listed in Table 10;
classifying the patient as suffering from the cancer in response to the tissue sample containing an amount of the citrullinated protein(s) equal to or greater than a threshold; and
classifying the patient as not suffering from the cancer in response to the tissue sample containing an amount of the citrullinated protein(s) less than the threshold.

11. The method of claim 10, wherein:
the cancer is selected from the group consisting of breast cancers, lung cancers, skin cancers, endometrial cancers, ovarian cancers, and colorectal cancers.

12. The method of claim 11, wherein:
the citrullinated protein(s) is/are selected from the group consisting of citrullinated vimentin and citrullinated α-enolase.

13. The method of claim 10, wherein the assaying comprises lysing cells of the tissue sample, to yield a tissue sample cell lysate, and isolating a protein fraction from the tissue sample cell lysate.

14. The method of claim 10, wherein the assaying comprises liquid chromatography-mass spectrometry (LC-MS).

15. A kit, comprising:
a cell lysis agent; and
instructions for performing a method, comprising instructions to:
provide a tissue sample from a patient suffering from a cancer or suspected of suffering from the cancer;
lyse cells of the tissue sample by exposing the tissue sample to the cell lysis agent;
assay the tissue sample for at least one citrullinated protein selected from the group consisting of the proteins encoded by genes listed in Table 1, the proteins encoded by genes listed in Table 2, and the proteins encoded by genes listed in Table 10;
classify the patient as suffering from the cancer, in response to the tissue sample containing an amount of the citrullinated protein(s) equal to or greater than a threshold; and
classify the patient as not suffering from the cancer, in response to the tissue sample containing an amount of the citrullinated protein(s) less than the threshold.

16. The kit of claim 15, wherein:
the cancer is selected from the group consisting of breast cancers, lung cancers, skin cancers, endometrial cancers, ovarian cancers, and colorectal cancers.

17. The kit of claim 16, wherein:
the citrullinated protein(s) is/are selected from the group consisting of citrullinated vimentin and citrullinated α-enolase.

18. The kit of claim 15, wherein the assaying comprises liquid chromatography-mass spectrometry (LC-MS).

19. A method, comprising:
providing a tumor sample from a patient suffering from a cancer;
assaying the tumor sample for at least one citrullinated amino acid sequence selected from the group consisting of sequences and corresponding modifications listed in Table 2, Table 9, and/or Table 10, and sequences having at least 70% identity to the sequences listed in Table 2, Table 9, and/or Table 10 and comprising at least one arginine residue; and
presenting to the immune system of the patient at least one peptide, wherein each peptide comprises at least one of the citrullinated amino acid sequences, in response to the tumor sample containing an amount of the citrullinated amino acid sequence(s) equal to or greater than a threshold.

20. The method of claim 19, wherein:
the cancer is selected from the group consisting of breast cancers, lung cancers, skin cancers, endometrial cancers, ovarian cancers, and colorectal cancers.

21. The method of claim 20, wherein:
the citrullinated amino acid sequences(s) is/are selected from the group consisting of
(SEQ ID NO: 43)
GVMVSHR*SGETEDTF,
(SEQ ID NO: 44)
LAQANGWGVMVSHR*SGETEDTF,
and
(SEQ ID NO: 45)
AVEKGVPLYR*HIADLAGNS, wherein R* is citrulline.

22. The method of claim 19, wherein the assaying comprises lysing cells of the tumor sample, to yield a tumor sample cell lysate, and quantifying the amount of the citrullinated amino acid sequence(s) in the tumor sample cell lysate.

23. The method of claim 19, further comprising:
administering, to the patient, an additional cancer therapy selected from the group consisting of surgical resection, chemotherapy, monoclonal antibody therapy, checkpoint inhibitor therapy, oncolytic virus therapy, thermal therapy, radiotherapy, and two or more thereof.

24. A kit, comprising:
at least one peptide, wherein each peptide comprises at least one citrullinated amino acid sequence selected from the group consisting of sequences and corresponding modifications listed in Table 2, Table 9, and/or Table 10, and sequences having at least 70% identity to the sequences listed in Table 2, Table 9, and/or Table 10 and comprising at least one arginine residue; and
instructions for performing a method, comprising instructions to:
provide a tumor sample from a patient suffering from a cancer;
assay the tumor sample for the citrullinated amino acid sequence(s) contained in each of the peptide(s); and
present to the immune system of the patient one or more of the peptides, in response to the tumor sample containing an amount equal to or greater than a threshold of the citrullinated amino acid sequence contained in the peptide(s).

25. The kit of claim 24, wherein:
the cancer is selected from the group consisting of breast cancers, lung cancers, skin cancers, endometrial cancers, ovarian cancers, and colorectal cancers.

26. The kit of claim 25, wherein:
the citrullinated amino acid sequences(s) is/are selected from the group consisting of
(SEQ ID NO: 43)
GVMVSHR*SGETEDTF,
(SEQ ID NO: 44)
LAQANGWGVMVSHR*SGETEDTF,
and
(SEQ ID NO: 45)
AVEKGVPLYR*HIADLAGNS, wherein R* is citrulline.

27. The kit of claim 24, further comprising a cell lysis agent, and wherein the instructions comprise instructions to assay the tumor sample by lysing cells of the tumor sample, to yield a tumor sample cell lysate, and quantifying the amount of the citrullinated amino acid sequence(s) in the tumor sample cell lysate.

28. The kit of claim 24, wherein the instructions further comprise instructions to:
administer, to the patient, an additional cancer therapy selected from the group consisting of surgical resection, chemotherapy, monoclonal antibody therapy, checkpoint inhibitor therapy, oncolytic virus therapy, thermal therapy, radiotherapy, and two or more thereof.

29. The kit of claim 28, wherein the additional cancer therapy is selected from the group consisting of chemotherapy, monoclonal antibody therapy, checkpoint inhibitor therapy, and oncolytic virus therapy, and the kit further comprises one or more of a chemotherapeutic agent, a monoclonal antibody, a checkpoint inhibitor, or an oncolytic virus.

30. A method, comprising:
providing a tumor sample from a patient suffering from a cancer;
assaying the tumor sample for a citrullinated protein encoded by a gene selected from the group consisting of genes listed in Table 1 as having a plasma membrane location; and
administering to the patient an anti-cancer agent targeting the citrullinated protein, in response to the tumor sample containing an amount of the citrullinated protein equal to or greater than a threshold.

31. The method of claim 30, wherein:
the cancer is selected from the group consisting of breast cancers, lung cancers, skin cancers, endometrial cancers, ovarian cancers, and colorectal cancers.

32. The method of claim 30, wherein the assaying comprises lysing cells of the tumor sample, to yield a tumor sample cell lysate, and quantifying the amount of the citrullinated amino acid sequence(s) in the tumor sample cell lysate.

33. The method of claim 30, further comprising:
administering, to the patient, an additional cancer therapy not targeting the citrullinated protein, selected from the group consisting of surgical resection, chemotherapy, monoclonal antibody therapy, checkpoint inhibitor therapy, oncolytic virus therapy, thermal therapy, radiotherapy, and two or more thereof.

34. A kit, comprising:
an anti-cancer agent targeting a citrullinated protein encoded by a gene selected from the group consisting of genes listed in Table 1 as having a plasma membrane location; and
instructions for performing a method, comprising instructions to:
provide a tumor sample from a patient suffering from a cancer;
assay the tumor sample for the citrullinated protein; and
administer to the patient an anti-cancer agent targeting the citrullinated protein, in response to the tumor sample containing an amount of the citrullinated protein equal to or greater than a threshold.

35. The kit of claim 34, wherein:
the cancer is selected from the group consisting of breast cancers, lung cancers, skin cancers, endometrial cancers, ovarian cancers, and colorectal cancers.

36. The kit of claim 34, further comprising a cell lysis agent, and wherein the instructions comprise instructions to assay the tumor sample by lysing cells of the tumor sample, to yield a tumor sample cell lysate, and quantifying the amount of the citrullinated amino acid sequence(s) in the tumor sample cell lysate.

37. The kit of claim 34, wherein the instructions further comprise instructions to:
administer, to the patient, an additional cancer therapy selected from the group consisting of surgical resection, chemotherapy, monoclonal antibody therapy, checkpoint inhibitor therapy, oncolytic virus therapy, thermal therapy, radiotherapy, and two or more thereof.

38. The kit of claim 37, wherein the additional cancer therapy is selected from the group consisting of chemotherapy, monoclonal antibody therapy, checkpoint inhibitor therapy, and oncolytic virus therapy, and the kit further comprises one or more of a chemotherapeutic agent, a monoclonal antibody, a checkpoint inhibitor, or an oncolytic virus.

39. An isolated peptide comprising at least 70% sequence identity to a peptide selected from the group consisting of peptides listed in Table 2, Table 9, and Table 10.

40. The peptide of claim 39, wherein the peptide comprises at least 6 contiguous amino acids of the peptide selected from the group consisting of peptides listed in Table 2, Table 9, and Table 10.

41. The peptide of claim 39, wherein the peptide is 15 amino acids or fewer in length.

42. The peptide of claim 39, wherein the peptide is 9 amino acids in length.

43. The peptide of claim 39, wherein the peptide is 15 amino acids in length.

44. The peptide of claim 39, wherein the peptide is 20-25 amino acids.

45. The peptide of claim 39, wherein the peptide is 22-24 amino acids.

46. The peptide of any one of claims 39-45, wherein the peptide is immunogenic.

47. The peptide of any one of claims 39-45, wherein the peptide is modified.

48. The peptide of claim 47, wherein the modification comprises conjugation to a molecule.

49. The peptide of claim 48, wherein the molecule comprises an antibody, a lipid, an adjuvant, or a detection moiety.

50. The peptide of any of claims 39-45, wherein the peptide has at least 90% sequence identity to the peptide selected from the group consisting of peptides listed in Table 2, Table 9, and Table 10.

51. The peptide of any of claims 39-45, wherein the peptide has 1, 2 or 3 substitutions relative to the peptide selected from the group consisting of peptides listed in Table 2, Table 9, and Table 10.

52. The peptide of any one of claims 39-45, wherein the peptide comprises 100% sequence identity to the peptide selected from the group consisting of peptides listed in Table 2, Table 9, and Table 10.
